(19) United States
(12) Patent Application Publication
(10) Pub. No.: US 2026/0232621 A1
(43) Pub. Date: Aug. 13, 2026

(54) METHODS OF TREATMENT USING LOOP DIURETICS

(71) Applicant: scPharmaceuticals Inc., Burlington, MA (US)

(72) Inventors: JENNIFER VANDIVER, Burlington, MA (US); JOHN F. MOHR, III, Burlington, MA (US); MICHAEL HASSMAN, Burlington, MA (US)

(21) Appl. No.: 19/146,305

(22) PCT Filed: Jan. 8, 2024
(86) PCT No.: PCT/US2024/010633

§ 371 Date: Jul. 8, 2025

Related U.S. Application Data
(60) Provisional application No. 63/479,093, filed on Jan. 9, 2023.

(51) Int. Cl. A61K 31/341; A61K 9/00; A61P 7/10

(52) U.S. Cl. CPC A61K 31/341; A61K 9/0019; A61P 7/10

(57) ABSTRACT

Disclosed herein, in part, are methods administering liquid pharmaceutical formulations containing a loop diuretic to a patient via subcutaneous injection. Methods of treating conditions such as congestion due to fluid overload, edema, and hypertension in a patient in need thereof are also provided.

### CROSS-REFERENCE TO RELATED APPLICATION

This application claims the benefit of and priority to U.S. Provisional Patent Application No. 63/479,093, filed on Jan. 9, 2023, the contents of which are incorporated herein in their entirety.

### BACKGROUND

Loop diuretics, can be used in the treatment of hypertension, edema and related conditions, including congestive heart failure. For example, furosemide, is commonly used in the treatment and/or management of edema associated with congestive heart failure, hepatic failure and renal disease. H. Bundgaard, T. Norgaard, N. M. Nielsen, “Photodegradation and hydrolysis of furosemide and furosemide esters in aqueous solutions,” International Journal of Pharmaceutics 42, 217 (1988).

Oral bioavailability, and therefore oral efficacy, of loop diuretics, such as furosemide, is often limited. Furosemide is commonly administered both parenterally and orally, although highly variable oral absorption is observed due to the combined effects of limited solubility and decreased stability at acidic pH. B. Devarakonda, D. P. Otto, A. Judefeind, R. A. Hill, M. M. de Villiers, “Effect of pH on the solubility and release of furosemide from polyamidoamine (PAMAM) dendrimer complexes,” International Journal of Pharmaceutics 345, 142 (Dec. 10, 2007). Accordingly, furosemide typically is administered intravenously or intramuscularly for most patients with congestive heart failure or other forms of more advanced edema.

Intravenous administration of a pharmaceutical drug, such as a loop diuretic, requires a trained healthcare professional for placement of the catheter and administration of the drug solution. In contrast, a pharmaceutical drug can be administered by subcutaneous injections or infusions by the patient or caregiver with the aid of auto-injection devices and/or wearable, on-body delivery infusion devices, for example, at home. Subcutaneous administration of loop diuretics by the patient or caregiver also can allow for more optimal therapeutic administration and total dose to provide a more appropriate pharmacokinetic and pharmacodynamic profile and patient outcome.

For subcutaneous administration, discomfort and pain during administration should be minimized so as to avoid poor patient compliance with the treatment regimen. Factors that can contribute to pain and discomfort perceived by a patient upon, during, or after subcutaneous administration include the injection volume, the pH of the formulation, and the osmolarity or tonicity of the formulation. Moreover, such a formulation should be stable in solution so that it readily is available for use and/or can be pre-loaded into a variety of drug delivery devices.

Therefore, a need exists for improved pharmaceutical formulations containing a loop diuretic (e.g., bumetanide, furosemide, or torsemide) that are chemically and physically stable over their shelf-life, contain a sufficient concentration of the loop diuretic (e.g., an effective amount in a suitable volume), and are at an appropriate pH and osmolality, for example, to permit subcutaneous administration (e.g., subcutaneous injection) of the loop diuretic.

### SUMMARY

In one aspect, the invention provides methods of treating a condition selected from the group consisting of congestion due to fluid overload, edema, and hypertension, in a human in need thereof, wherein the methods generally comprise administering a liquid pharmaceutical formulation comprising a loop diuretic. In certain embodiments, an effective amount of the loop diuretic is administered by subcutaneous injection. That is, it has been discovered that liquid pharmaceutical formulations of the invention, e.g., about 0.5 mL to about 1 mL in total volume, including an effective amount of the loop diuretic, e.g., about 1 mg to about 100 mg, can be delivered subcutaneously over reduced time periods, such as equal to or less than 30 secs. Delivery of 80 mg furosemide in a volume of about 1 mL or less during reduced time periods without substantial increase in pain at the site of injection can permit a patient to use a subcutaneous injection device (e.g., an autoinjector) or the like for shorter periods of time, which should increase compliance with the treatment.

In various embodiments of the invention, the method comprises administering to the human via subcutaneous injection an effective amount of a loop diuretic in a total volume of about 1 mL or less of a liquid pharmaceutical formulation.

In certain embodiments, the effective amount of the loop diuretic in the total volume of the liquid pharmaceutical formulation is about 1 mg to about 100 mg. In certain embodiments, the concentration of the loop diuretic in the total volume of the liquid pharmaceutical formulation is about 2 mg/mL to about 100 mg/mL.

In certain embodiments, the total urine output of the human about 6 hours after administration is complete is about 600 mL to about 5000 mL. In certain embodiments, the total urine output of the human about 8 hours after administration is complete is about 600 mL to about 5300 mL. In certain embodiments, the total urine output of the human about 12 hours after administration is complete is about 600 mL to about 5500 mL.

In certain embodiments, the loop diuretic is bumetanide, furosemide, or torsemide.

In various embodiments of the invention, the method comprises administering to the human via subcutaneous injection an effective amount of furosemide in a total volume of about 1 mL or less of a liquid pharmaceutical formulation.

In certain embodiments, the effective amount of furosemide in the total volume of the liquid pharmaceutical formulation is about 40 mg to about 80 mg. In certain embodiments, the concentration of furosemide in the total volume of the liquid pharmaceutical formulation is about 80 mg/mL. In certain embodiments, the total volume of the liquid pharmaceutical formulation is about 0.5 mL or about 1 mL.

In various embodiments of the invention, the method comprises administering to a human via subcutaneous injection about 80 mg of furosemide in a total volume of about 1 mL of a liquid pharmaceutical formulation.

In various embodiments of the invention, the method comprises administering to a human via subcutaneous injection about 40 mg of furosemide in a total volume of about 0.5 mL of a liquid pharmaceutical formulation.

In certain embodiments, the method provides a furosemide Cₘₐₓ in plasma of the human of about 1500 ng/mL to about 9800 ng/mL. In certain embodiments, the method provides a furosemide AUCₗₐₛₜ in plasma of the human of about 4400 h*ng/mL to about 31700 h*ng/ml. In certain embodiments, the method provides a furosemide AUC_{inf} in plasma of the human of about 4000 h*ng/ml to about 24000 h*ng/mL. In certain embodiments, the total urine output of the human about 6 hours after administration is complete is about 600 mL to about 5000 mL. In certain embodiments, the total urine output of the human about 8 hours after administration is complete is about 600 mL to about 5300 mL. In certain embodiments, the total urine output of the human about 12 hours after administration is complete is about 600 mL to about 5500 mL.

In various embodiments of the invention, the method comprises administering to a human via subcutaneous injection about 80 mg furosemide in a total volume of about 1 mL of a liquid pharmaceutical formulation, wherein one or more of the following:
the method provides a furosemide Cₘₐₓ in plasma of the human of about 3000 ng/ml to about 9800 ng/ml;
the method provides a furosemide AUCₗₐₛₜ in plasma of the human of about 8800 h*ng/ml to about 31700 h*ng/mL;
the method provides a furosemide AUC_{inf} in plasma of the human of about 8900 h*ng/ml to about 33400 h*ng/ml;
the total urine output of the human about 6 hours after administration is complete is about 1200 mL to about 5000 mL;
the total urine output of the human about 8 hours after administration is complete is about 1200 mL to about 5300 mL; and
the total urine output of the human about 12 hours after administration is complete is about 1200 mL to about 5500 mL.

In various embodiments of the invention, the method comprises administering to a human via subcutaneous injection about 80 mg furosemide in a total volume of about 1 mL of a liquid pharmaceutical formulation, wherein:
the method provides a furosemide Cₘₐₓ in plasma of the human of about 3000 ng/ml to about 9800 ng/ml;
the method provides a furosemide AUCₗₐₛₜ in plasma of the human of about 8800 h*ng/ml to about 31700 h*ng/ml;
the method provides a furosemide AUC_{inf} in plasma of the human of about 8900 h*ng/ml to about 33400 h*ng/ml;
the total urine output of the human about 6 hours after administration is complete is about 1200 mL to about 5000 mL;
the total urine output of the human about 8 hours after administration is complete is about 1200 mL to about 5300 mL; and
the total urine output of the human about 12 hours after administration is complete is about 1200 mL to about 5500 mL.

In various embodiments of the invention, the method comprises administering to a human via subcutaneous injection about 40 mg furosemide in a total volume of about 0.5 mL of a liquid pharmaceutical formulation, wherein one or more of the following:
the method provides a furosemide Cₘₐₓ in plasma of the human of about 1500 ng/mL to about 4900 ng/ml;
the method provides a furosemide AUCₗₐₛₜ in plasma of the human of about 4400 h*ng/ml to about 15850 h*ng/mL;
the method provides a furosemide AUC_{inf} in plasma of the human of about 4450 h*ng/ml to about 16700 h*ng/ml;
the total urine output of the human about 6 hours after administration is complete is about 600 mL to about 2500 mL;
the total urine output of the human about 8 hours after administration is complete is about 600 mL to about 2650 mL; and
the total urine output of the human about 12 hours after administration is complete is about 600 mL to about 2750 mL.

In various embodiments of the invention, the method comprises administering to a human via subcutaneous injection about 40 mg furosemide in a total volume of about 0.5 mL of a liquid pharmaceutical formulation, wherein:
the method provides a furosemide Cₘₐₓ in plasma of the human of about 1500 ng/ml to about 4900 ng/ml;
the method provides a furosemide AUCₗₐₛₜ in plasma of the human of about 4400 h*ng/ml to about 15850 h*ng/ml;
the method provides a furosemide AUC_{inf} in plasma of the human of about 4450 h*ng/ml to about 16700 h*ng/ml;
the total urine output of the human about 6 hours after administration is complete is about 600 mL to about 2500 mL;
the total urine output of the human about 8 hours after administration is complete is about 600 mL to about 2650 mL; and
the total urine output of the human about 12 hours after administration is complete is about 600 mL to about 2750 mL.

In certain embodiments, administering comprises administering the total volume over a period of equal to or less than 30 secs. In certain embodiments, administering comprises administering the total volume from an autoinjector.

In certain embodiments, the liquid pharmaceutical formulation further comprises a pharmaceutically acceptable buffer, optionally wherein the pharmaceutically acceptable buffer comprises tromethamine, or a pharmaceutically acceptable salt thereof. In certain embodiments, the liquid pharmaceutical formulation has a pH of about 7.2 to about 8. In certain embodiments, the liquid pharmaceutical formulation further comprises one or more pharmaceutically acceptable excipients, optionally wherein the one or more pharmaceutically acceptable excipients is selected from benzyl alcohol, sodium hydroxide, sodium chloride, hydrochloric acid, and combinations thereof.

In various embodiments, the human is an adult human, optionally wherein the adult human is 45 to 80 years of age. In certain embodiments, the adult human has New York Heart Association (NYHA) Class II, Class III or Class IV chronic heart failure, liver disease or impairment, or renal disease or impairment. In certain embodiments, the adult human displays reduced responsiveness to oral diuretics before beginning subcutaneous administration according to the method. In certain embodiments, the edema is associated with congestive heart failure, cirrhosis of the liver, or renal disease, optionally wherein the renal disease is nephrotic syndrome.

### DETAILED DESCRIPTION

It has now been discovered that an effective amount of a loop diuretic in a volume of about 1 mL or less can be delivered subcutaneously during reduced time periods without substantial increase in pain at the site of injection. Such methods can permit a patient to use a subcutaneous injection device (e.g., an autoinjector) or the like for shorter periods of time, which should increase compliance with the treatment. As generally described herein, the invention provides methods of treating various conditions, diseases, and disorders (e.g., congestion due to fluid overload, edema, and hypertension) in a patient (human) in need thereof. The methods generally can comprise subcutaneously administering a liquid pharmaceutical formulation of furosemide (e.g., 80 mg in 1 mL) to the patient over a period of equal to or less than 30 seconds. The liquid pharmaceutical formulations of furosemide and delivery profiles described herein can result in optimized furosemide pharmacokinetic and pharmacodynamic profiles that can maximize patient outcomes (e.g., total urine output).

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

The terms “a” and “an” as used herein mean “one or more” and include the plural unless the context is inappropriate.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10%, ±5%, ±3%, ±2%, or ±1% variation from the nominal value unless otherwise indicated or inferred from the context.

At various places in the present specification, values are disclosed in groups or in ranges. It is specifically intended that the description include each and every individual subcombination of the members of such groups and ranges. For example, a number in the range of 0 to 40 is specifically intended to individually disclose the integers 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and a number in the range of 1 to 20 is specifically intended to individually disclose the integers 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As used herein, a “compound” (including a specifically named compound, e.g., furosemide, bumetanide, ethacrynic acid, torsemide) refers to the compound itself and its pharmaceutically acceptable salts unless otherwise understood from the context of the description or expressly limited to one particular form of the compound, e.g., the compound itself, or a pharmaceutically acceptable salt thereof.

As used herein, “furosemide” refers to a compound having the formula:

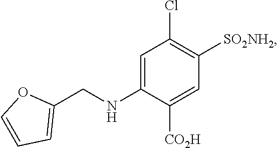 [SMILES: NS(=O)(=O)c1cc(C(=O)O)c(NCc2ccco2)cc1Cl]

and pharmaceutically acceptable salts thereof. Such salts may include, but are not limited to, furosemide sodium salt and furosemide quaternary ammonium salt. Furosemide may be referred to by other names, for example, frusemide, 5-(aminosulphonyl)-4-chloro-2-[(2-furanyl-methyl)amino]benzoic acid, or its IUPAC name, 4-chloro-2-(furan-2-ylmethylamino)-5-sulfamoyl-benzoic acid, or its common trade name, Lasix®.

As used herein, “bumetanide” refers to a compound having the formula:

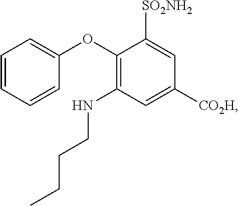 [SMILES: CCCCNc1cc(C(=O)O)cc(S(N)(=O)=O)c1Oc1ccccc1]

and pharmaceutically acceptable salts thereof. Such salts may include, but are not limited to, furosemide potassium salt and furosemide sodium salt. Bumetanide may be referred to by other names, for example, bumedyl, bumethanide, or its IUPAC name, 3-(butylamino)-4-phenoxy-5-sulfamoylbenzoic acid, or its common trade names, Bumex® and Burinex®.

As used herein, “ethacrynic acid” refers to a compound having the formula:

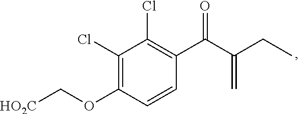 [SMILES: C=C(CC)C(=O)c1ccc(OCC(=O)O)c(Cl)c1Cl]

and pharmaceutically acceptable salts thereof. Such salts may include, but are not limited to, ethacrynic acid sodium salt (also referred to as ethacrynate sodium). Ethacrynic acid may be referred to by other names, for example, etacrynic acid, or its IUPAC name, 2-[2,3-dichloro-4-(2-methylidenebutanoyl) phenoxy]acetic acid, or its common trade names, Sodium Edecrin® and Edecrin®.

As used herein, “torsemide” refers to a compound having the formula:

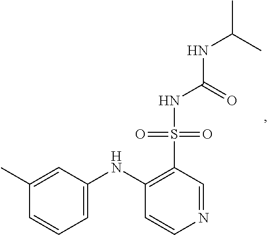 [SMILES: Cc1cccc(Nc2ccncc2S(=O)(=O)NC(=O)NC(C)C)c1]

and pharmaceutically acceptable salts thereof. Such salts may include, but are not limited to, torsemide sodium salt. Torsemide may be referred to by other names, for example, torasemide, 1-isopropyl-3-((4-(3-methylphenylamino)pyridine)-3-sulfonyl) urea, 1-isopropyl-3-((4-m-toluidino-3-pyridyl) sulfonyl) urea, or its IUPAC name, 1-[4-(3-methylanilino)pyridin-3-yl]sulfonyl-3-propan-2-ylurea, or its common trade name, Demadex®.

As used herein, the terms “subject” and “patient” refer to organisms to be treated by the methods and/or compositions described herein. Such organisms are preferably mammals (e.g., murines, simians, equines, bovines, porcines, canines, felines, and the like), and more preferably humans.

As used herein, a “buffer” refers to an aqueous solution that is resistant to changes in pH. A buffer may include a “buffering agent” such as a weak acid and its salt, or a weak base and its salt, which assist in maintaining the stability of the pH. Examples of buffers used in pharmaceutical formulations include bicarbonate buffers, carbonate buffers, citrate buffers, histidine buffers, phosphate buffers, tartrate buffers, tris(hydroxymethyl)aminomethane (or 2-amino-2-hydroxymethyl-propane-1,3-diol [(HOCH₂)₃CNH₂]) buffers, and combinations thereof. Certain of these buffers are suitable for pharmaceutical formulations administered subcutaneously.

Tris(hydroxymethyl)aminomethane or a tris(hydroxymethyl)aminomethane buffer can be referred to as “TRIS,” “Tris,” “Tris buffer,” “Trisamine,” “THAM,” “tromethamine,” and other names. In addition, many buffers and/or buffer systems can include Tris, or a pharmaceutically acceptable salt thereof, and can be used in the present teachings. For example, Tris-buffered saline (“TBS”), Tris-hydrochloride buffer (“Tris-HCl”), Tris base (pH 10.6), Tris/borate/ethylene diamine tetra-acetate (“EDTA”) buffer (“TBE”), and Tris/acetate/EDTA buffer (“TAE”). Tris base often is used with Tris-HCl to prepare Tris buffers at a desired pH. In addition, the present teachings can include Tris-related compounds, for example, compounds derived from Tris or structurally-related to Tris, that can act as a buffer.

As used herein, “tonicity” refers to the ionic strength or concentration of ions in a solution such as a pharmaceutical formulation. Tonicity often is measured in molarity (“M”). As used herein, an “isotonic solution,” an “isotonic formulation,” an “isotonic pharmaceutical formulation,” and a pharmaceutical formulation that is “isotonic” refers to a solution or formulation that has the same or similar concentration of ions as found in bodily fluids.

As used herein, “physiological pH” refers to a pH of about 7.4.

As used herein, “osmoticity,” “osmolarity,” and “osmolality” refer to the osmotic pressure of a solution such as a pharmaceutical formulation. Osmoticity often is measured in osmolarity (“Osm/L” or “OsM”) or osmolality (“Osm/kg”). When measuring freezing point depression, the observed value is the osmolality of the solution. In contrast to tonicity, osmoticity accounts for un-ionized solutes in a solution such that when present, the osmolarity or osmolality of the solution will be higher than its tonicity. The osmolarity of a liquid pharmaceutical formulation described herein can be measured, for example, using a vapor pressure method.

As used herein, “osmolarity adjustor,” “osmolarity adjustor,” and “osmotic agent” refer to a pharmaceutically acceptable compound that may be added to a liquid pharmaceutical formulation described herein in order to modulate the osmolarity of the liquid pharmaceutical formulation.

As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. In certain embodiments, supplementary active ingredients can also be incorporated into the pharmaceutical compositions.

As used herein, “pharmaceutically acceptable excipient” refers to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxypropylmethylcellulose, polyvinyl pyrrolidine, and colors, and the like. Such preparations can be sterilized and, if desired, mixed with auxiliary agents such as lubricants, preservatives, stabilizers, surfactants (e.g., polysorbate 20 or polysorbate 80), wetting agents, emulsifiers, salts for influencing osmotic pressure, buffers, coloring, and/or aromatic substances and the like that do not deleteriously react with the compounds of the invention. For examples of excipients and carriers, see Martin, Remington's Pharmaceutical Sciences, 15th Ed., Mack Publ. Co., Easton, PA (1975).

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin, Remington's Pharmaceutical Sciences, 15th Ed., Mack Publ. Co., Easton, PA (1975).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

As used herein, the term “effective amount” refers to the amount of a composition (e.g., a liquid pharmaceutical formulation of the present invention) sufficient to effect beneficial or desired results, including a desired therapeutic effect. An effective amount can be administered in one or more administrations, applications or dosages and is not intended to be limited to a particular formulation or administration route.

As used herein, the terms “treat,” “treating,” and “treatment” include any effect, e.g., lessening, reducing, modulating, ameliorating or eliminating, that results in the improvement of the condition, disease, disorder, and the like, or ameliorating a symptom thereof.

As used herein, the term “congestion” (in heart failure) is the presence of signs and symptoms of extracellular fluid accumulation that results in increased cardiac filling pressures leading to reduced cardiac output. This reduced cardiac output is further exacerbated by neurohormonal activation leading to increased renal sodium and water avidity resulting in an increased plasma volume.

As used herein, “fluid overload,” “volume overload” and “hypervolemia”, may describe a medical condition where there is too much fluid in the blood. Excess fluid, primarily salt and water, may build up throughout the body resulting in weight gain.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### Liquid Pharmaceutical Formulations of Loop Diuretics

As described herein, in one aspect, the invention provides liquid pharmaceutical formulations comprising an effective amount of a loop diuretic, or a pharmaceutically acceptable salt thereof, for the treatment of a condition described herein. In certain embodiments, the condition is selected from the group consisting of congestion due to fluid overload, edema, and hypertension, and combinations thereof. In certain embodiments, the condition is congestion due to fluid overload. In certain embodiments, the condition is edema. In certain embodiments, the condition is hypertension.

### (A) Loop Diuretics

In certain embodiments, the effective amount of the loop diuretic in a liquid pharmaceutical formulation described herein is about 1 mg to about 100 mg, about 5 mg to about 100 mg, about 10 mg to about 100 mg, about 20 mg to about 100 mg, about 30 mg to about 100 mg, about 40 mg to about 100 mg, about 50 mg to about 100 mg, about 60 mg to about 100 mg, about 70 mg to about 100 mg, about 80 mg to about 100 mg, about 90 mg to about 100 mg, about 1 mg to about 90 mg, about 1 mg to about 80 mg, about 1 mg to about 70 mg, about 1 mg to about 60 mg, about 1 mg to about 50 mg, about 1 mg to about 40 mg, about 1 mg to about 30 mg, about 1 mg to about 20 mg, about 1 mg to about 10 mg, about 1 mg to about 5 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 10 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 90 mg, about 40 mg to about 80 mg, about 40 mg to about 70 mg, about 40 mg to about 60 mg, about 40 mg to about 50 mg, about 50 mg to about 90 mg, about 50 mg to about 80 mg, about 50 mg to about 70 mg, about 50 mg to about 60 mg, about 60 mg to about 90 mg, about 60 mg to about 80 mg, about 60 mg to about 70 mg, about 70 mg to about 90 mg, about 70 mg to about 80 mg, or about 80 mg to about 90 mg. In certain embodiments, the effective amount of the loop diuretic in a liquid pharmaceutical formulation described herein is about 1 mg to about 100 mg. In certain embodiments, the effective amount of the loop diuretic in a liquid pharmaceutical formulation described herein is about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, or about 100 mg.

In certain embodiments, the concentration of the loop diuretic in a liquid pharmaceutical formulation described herein can be about 2 mg/mL to about 100 mg/mL, about 5 mg/mL to about 100 mg/mL, about 10 mg/mL to about 100 mg/mL, about 20 mg/mL to about 100 mg/mL, about 30 mg/mL to about 100 mg/mL, about 40 mg/mL to about 100 mg/mL, about 50 mg/mL to about 100 mg/mL, about 60 mg/mL to about 100 mg/mL, about 70 mg/mL to about 100 mg/mL, about 80 mg/mL to about 100 mg/mL, about 90 mg/mL to about 100 mg/mL, about 2 mg/mL to about 90 mg/mL, about 2 mg/mL to about 80 mg/mL, about 2 mg/mL to about 70 mg/mL, about 2 mg/mL to about 60 mg/mL, about 2 mg/mL to about 50 mg/mL, about 2 mg/mL to about 40 mg/mL, about 2 mg/mL to about 30 mg/mL, about 2 mg/mL to about 20 mg/mL, about 2 mg/mL to about 10 mg/mL, about 2 mg/mL to about 5 mg/mL, about 5 mg/mL to about 90 mg/mL, about 5 mg/mL to about 80 mg/mL, about 5 mg/mL to about 70 mg/mL, about 5 mg/mL to about 60 mg/mL, about 5 mg/mL to about 50 mg/mL, about 5 mg/mL to about 40 mg/mL, about 5 mg/mL to about 30 mg/mL, about 5 mg/mL to about 20 mg/mL, about 5 mg/mL to about 10 mg/mL, about 10 mg/mL to about 90 mg/mL, about 10 mg/mL to about 80 mg/mL, about 10 mg/mL to about 70 mg/mL, about 10 mg/mL to about 60 mg/mL, about 10 mg/mL to about 50 mg/mL, about 10 mg/mL to about 40 mg/mL, about 10 mg/mL to about 30 mg/mL, about 10 mg/mL to about 20 mg/mL, about 20 mg/mL to about 90 mg/mL, about 20 mg/mL to about 80 mg/mL, about 20 mg/mL to about 70 mg/mL, about 20 mg/mL to about 60 mg/mL, about 20 mg/mL to about 50 mg/mL, about 20 mg/mL to about 40 mg/mL, about 20 mg/mL to about 30 mg/mL, about 30 mg/mL to about 90 mg/mL, about 30 mg/mL to about 80 mg/mL, about 30 mg/mL to about 70 mg/mL, about 30 mg/mL to about 60 mg/mL, about 30 mg/mL to about 50 mg/mL, about 30 mg/mL to about 40 mg/mL, about 40 mg/mL to about 90 mg/mL, about 40 mg/mL to about 80 mg/mL, about 40 mg/mL to about 70 mg/mL, about 40 mg/mL to about 60 mg/mL, about 40 mg/mL to about 50 mg/mL, about 50 mg/mL to about 90 mg/mL, about 50 mg/mL to about 80 mg/mL, about 50 mg/mL to about 70 mg/mL, about 50 mg/mL to about 60 mg/mL, about 60 mg/mL to about 90 mg/mL, about 60 mg/mL to about 80 mg/mL, about 60 mg/mL to about 70 mg/mL, about 70 mg/mL to about 90 mg/mL, about 70 mg/mL to about 80 mg/mL, or about 80 mg/mL to about 90 mg/mL. In certain embodiments, the concentration of the loop diuretic in a liquid pharmaceutical formulation described herein can be about 2 mg/ml to about 100 mg/mL.

In various embodiments, a liquid pharmaceutical formulation described herein may comprise a pharmaceutically acceptable salt of a loop diuretic.

In certain embodiments, a liquid pharmaceutical formulation described herein comprises a pharmaceutically acceptable salt of a loop diuretic in an amount that provides about 1 mg to about 100 mg, about 5 mg to about 100 mg, about 10 mg to about 100 mg, about 20 mg to about 100 mg, about 30 mg to about 100 mg, about 40 mg to about 100 mg, about 50 mg to about 100 mg, about 60 mg to about 100 mg, about 70 mg to about 100 mg, about 80 mg to about 100 mg, about 90 mg to about 100 mg, about 1 mg to about 90 mg, about 1 mg to about 80 mg, about 1 mg to about 70 mg, about 1 mg to about 60 mg, about 1 mg to about 50 mg, about 1 mg to about 40 mg, about 1 mg to about 30 mg, about 1 mg to about 20 mg, about 1 mg to about 10 mg, about 1 mg to about 5 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 10 mg, about mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 90 mg, about 40 mg to about 80 mg, about 40 mg to about 70 mg, about 40 mg to about 60 mg, about 40 mg to about 50 mg, about 50 mg to about 90 mg, about 50 mg to about 80 mg, about 50 mg to about 70 mg, about 50 mg to about 60 mg, about 60 mg to about 90 mg, about 60 mg to about 80 mg, about 60 mg to about 70 mg, about 70 mg to about 90 mg, about 70 mg to about 80 mg, or about 80 mg to about 90 mg of the loop diuretic in its free acid/free base form. In certain embodiments, a liquid pharmaceutical formulation described herein comprises a pharmaceutically acceptable salt of a loop diuretic in an amount that provides about 1 mg to about 100 mg of the loop diuretic in its free acid/free base form.

In certain embodiments, a liquid pharmaceutical formulation described herein comprises a pharmaceutically acceptable salt of a loop diuretic in an amount that provides about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, or about 100 mg of the loop diuretic in its free acid/free base form.

In certain embodiments, a liquid pharmaceutical formulation described herein comprises a pharmaceutically acceptable salt of a loop diuretic in an amount that provides a concentration of the loop diuretic in its free acid/free base form of about 2 mg/mL to about 100 mg/mL, about 5 mg/mL to about 100 mg/mL, about 10 mg/mL to about 100 mg/mL, about 20 mg/mL to about 100 mg/mL, about 30 mg/mL to about 100 mg/mL, about 40 mg/mL to about 100 mg/mL, about 50 mg/mL to about 100 mg/mL, about 60 mg/mL to about 100 mg/mL, about 70 mg/mL to about 100 mg/mL, about 80 mg/mL to about 100 mg/mL, about 90 mg/mL to about 100 mg/mL, about 2 mg/mL to about 90 mg/mL, about 2 mg/mL to about 80 mg/mL, about 2 mg/mL to about 70 mg/mL, about 2 mg/mL to about 60 mg/mL, about 2 mg/mL to about 50 mg/mL, about 2 mg/mL to about 40 mg/mL, about 2 mg/mL to about 30 mg/mL, about 2 mg/mL to about 20 mg/mL, about 2 mg/mL to about 10 mg/mL, about 2 mg/mL to about 5 mg/mL, about 5 mg/mL to about 90 mg/mL, about 5 mg/mL to about 80 mg/mL, about 5 mg/mL to about 70 mg/mL, about 5 mg/mL to about 60 mg/mL, about 5 mg/mL to about 50 mg/mL, about 5 mg/mL to about 40 mg/mL, about 5 mg/mL to about 30 mg/mL, about 5 mg/mL to about 20 mg/mL, about 5 mg/mL to about 10 mg/mL, about 10 mg/mL to about 90 mg/mL, about 10 mg/mL to about 80 mg/mL, about 10 mg/mL to about 70 mg/mL, about 10 mg/mL to about 60 mg/mL, about 10 mg/mL to about 50 mg/mL, about 10 mg/mL to about 40 mg/mL, about 10 mg/mL to about 30 mg/mL, about 10 mg/mL to about 20 mg/mL, about 20 mg/mL to about 90 mg/mL, about 20 mg/mL to about 80 mg/mL, about 20 mg/mL to about 70 mg/mL, about 20 mg/mL to about 60 mg/mL, about 20 mg/mL to about 50 mg/mL, about 20 mg/mL to about 40 mg/mL, about 20 mg/mL to about 30 mg/mL, about 30 mg/mL to about 90 mg/mL, about 30 mg/mL to about 80 mg/mL, about 30 mg/mL to about 70 mg/mL, about 30 mg/mL to about 60 mg/mL, about 30 mg/mL to about 50 mg/mL, about 30 mg/mL to about 40 mg/mL, about 40 mg/mL to about 90 mg/mL, about 40 mg/mL to about 80 mg/mL, about 40 mg/mL to about 70 mg/mL, about 40 mg/mL to about 60 mg/mL, about 40 mg/mL to about 50 mg/mL, about 50 mg/mL to about 90 mg/mL, about 50 mg/mL to about 80 mg/mL, about 50 mg/mL to about 70 mg/mL, about 50 mg/mL to about 60 mg/mL, about 60 mg/mL to about 90 mg/mL, about 60 mg/mL to about 80 mg/mL, about 60 mg/mL to about 70 mg/mL, about 70 mg/mL to about 90 mg/mL, about 70 mg/mL to about 80 mg/mL, or about 80 mg/mL to about 90 mg/mL. In certain embodiments, a liquid pharmaceutical formulation described herein comprises a pharmaceutically acceptable salt of a loop diuretic in an amount that provides a concentration of the loop diuretic in its free acid/free base form of about 2 mg/mL to about 100 mg/mL.

In certain embodiments, the loop diuretic is bumetanide, furosemide, or torsemide.

### (i) Furosemide

In various embodiments, the invention provides liquid pharmaceutical formulations comprising an effective amount of furosemide, or a pharmaceutically acceptable salt thereof, for the treatment of a condition described herein.

In certain embodiments, the effective amount of furosemide in a liquid pharmaceutical formulation described herein is about 40 mg to about 100 mg, about 45 mg to about 100 mg, about 50 mg to about 100 mg, about 55 mg to about 100 mg, about 60 mg to about 100 mg, about 65 mg to about 100 mg, about 70 mg to about 100 mg, about 75 mg to about 100 mg, about 80 mg to about 100 mg, about 85 mg to about 100 mg, about 90 mg to about 100 mg, about 95 mg to about 100 mg, about 40 mg to about 95 mg, about 40 mg to about 90 mg, about 40 mg to about 85 mg, about 40 mg to about 80 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 45 mg to about 95 mg, about 45 mg to about 90 mg, about 45 mg to about 85 mg, about 45 mg to about 80 mg, about 45 mg to about 75 mg, about 45 mg to about 70 mg, about 45 mg to about 65 mg, about 45 mg to about 60 mg, about 45 mg to about 55 mg, about 45 mg to about 50 mg, about 50 mg to about 95 mg, about 50 mg to about 90 mg, about 50 mg to about 85 mg, about 50 mg to about 80 mg, about 50 mg to about 75 mg, about 50 mg to about 70 mg, about 50 mg to about 65 mg, about 50 mg to about 60 mg, about 50 mg to about 55 mg, about 55 mg to about 95 mg, about 55 mg to about 90 mg, about 55 mg to about 85 mg, about 55 mg to about 80 mg, about 55 mg to about 75 mg, about 55 mg to about 70 mg, about 55 mg to about 65 mg, about 55 mg to about 60 mg, about 60 mg to about 95 mg, about 60 mg to about 90 mg, about 60 mg to about 85 mg, about 60 mg to about 80 mg, about 60 mg to about 75 mg, about 60 mg to about 70 mg, about 60 mg to about 65 mg, about 65 mg to about 95 mg, about 65 mg to about 90 mg, about 65 mg to about 85 mg, about 65 mg to about 80 mg, about 65 mg to about 75 mg, about 65 mg to about 70 mg, about 70 mg to about 95 mg, about 70 mg to about 90 mg, about 70 mg to about 85 mg, about 70 mg to about 80 mg, about 70 mg to about 75 mg, about 75 mg to about 95 mg, about 75 mg to about 90 mg, about 75 mg to about 85 mg, about 75 mg to about 80 mg, about 80 mg to about 95 mg, about 80 mg to about 90 mg, about 80 mg to about 85 mg, about 85 mg to about 95 mg, about 85 mg to about 90 mg, or about 90 mg to about 95 mg. In certain embodiments, the effective amount of furosemide in a liquid pharmaceutical formulation described herein is about 40 mg to about 100 mg. In certain embodiments, the effective amount of furosemide in a liquid pharmaceutical formulation described herein is about 40 mg to about 80 mg.

In various embodiments, the effective amount of furosemide in a liquid pharmaceutical formulation described herein is about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In certain embodiments, the effective amount of furosemide in a liquid pharmaceutical formulation described herein is about 40 mg. In certain embodiments, the effective amount of furosemide in a liquid pharmaceutical formulation described herein is about 80 mg.

In various embodiments, a liquid pharmaceutical formulation described herein comprises about 5% (w/w) to about 10% (w/w), about 6% (w/w) to about 10% (w/w), about 7% (w/w) to about 10% (w/w), about 8% (w/w) to about 10% (w/w), about 9% (w/w) to about 10% (w/w), about 5% (w/w) to about 9% (w/w), about 5% (w/w) to about 8% (w/w), about 5% (w/w) to about 7% (w/w), about 5% (w/w) to about 6% (w/w), about 6% (w/w) to about 9% (w/w), about 6% (w/w) to about 8% (w/w), about 6% (w/w) to about 7% (w/w), about 7% (w/w) to about 9% (w/w), about 7% (w/w) to about 8% (w/w), or about 8% (w/w) to about 9% (w/w) furosemide.

In certain embodiments, a liquid pharmaceutical formulation described herein comprises about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w) furosemide.

In certain embodiments, a liquid pharmaceutical formulation described herein comprises about 5% (w/w) furosemide. In certain embodiments, a liquid pharmaceutical formulation described herein comprises about 6% (w/w) furosemide. In certain embodiments, a liquid pharmaceutical formulation described herein comprises about 7% (w/w) furosemide. In certain embodiments, a liquid pharmaceutical formulation described herein comprises about 8% (w/w) furosemide. In certain embodiments, a liquid pharmaceutical formulation described herein comprises about 9% (w/w) furosemide. In certain embodiments, a liquid pharmaceutical formulation described herein comprises about 10% (w/w) furosemide.

In certain embodiments, the concentration of furosemide in a liquid pharmaceutical formulation described herein can be about 50 mg/mL to about 250 mg/mL, about 60 mg/mL to about 250 mg/mL, about 70 mg/mL to about 250 mg/mL, about 80 mg/mL to about 250 mg/mL, about 90 mg/mL to about 250 mg/mL, about 100 mg/mL to about 250 mg/mL, about 120 mg/mL to about 250 mg/mL, about 140 mg/mL to about 250 mg/mL, about 160 mg/mL to about 250 mg/mL, about 180 mg/mL to about 250 mg/mL, about 200 mg/mL to about 250 mg/mL, about 50 mg/mL to about 200 mg/mL, about 50 mg/mL to about 180 mg/mL, about 50 mg/mL to about 160 mg/mL, about 50 mg/mL to about 140 mg/mL, about 50 mg/mL to about 120 mg/mL, about 50 mg/mL to about 100 mg/mL, about 50 mg/mL to about 90 mg/mL, about 50 mg/mL to about 80 mg/mL, about 50 mg/mL to about 70 mg/mL, about 50 mg/mL to about 60 mg/mL, about 60 mg/mL to about 200 mg/mL, about 60 mg/mL to about 180 mg/mL, about 60 mg/mL to about 160 mg/mL, about 60 mg/mL to about 140 mg/mL, about 60 mg/mL to about 120 mg/mL, about 60 mg/mL to about 100 mg/mL, about 60 mg/mL to about 90 mg/mL, about 60 mg/mL to about 80 mg/mL, about 60 mg/mL to about 70 mg/mL, about 70 mg/mL to about 200 mg/mL, about 70 mg/mL to about 180 mg/mL, about 70 mg/mL to about 160 mg/mL, about 70 mg/mL to about 140 mg/mL, about 70 mg/mL to about 120 mg/mL, about 70 mg/mL to about 100 mg/mL, about 70 mg/mL to about 90 mg/mL, about 70 mg/mL to about 80 mg/mL, about 80 mg/mL to about 200 mg/mL, about 80 mg/mL to about 180 mg/mL, about 80 mg/mL to about 160 mg/mL, about 80 mg/mL to about 140 mg/mL, about 80 mg/mL to about 120 mg/mL, about 80 mg/mL to about 100 mg/mL, about 80 mg/mL to about 90 mg/mL, about 90 mg/mL to about 200 mg/mL, about 90 mg/mL to about 180 mg/mL, about 90 mg/mL to about 160 mg/mL, about 90 mg/mL to about 140 mg/mL, about 90 mg/mL to about 120 mg/mL, about 90 mg/mL to about 100 mg/mL, about 100 mg/mL to about 200 mg/mL, about 100 mg/mL to about 180 mg/mL, about 100 mg/mL to about 160 mg/mL, about 100 mg/mL to about 140 mg/mL, about 100 mg/mL to about 120 mg/mL, about 120 mg/mL to about 200 mg/mL, about 120 mg/mL to about 180 mg/mL, about 120 mg/mL to about 160 mg/mL, about 120 mg/mL to about 140 mg/mL, about 140 mg/mL to about 200 mg/mL, about 140 mg/mL to about 180 mg/mL, about 140 mg/mL to about 160 mg/mL, about 160 mg/mL to about 200 mg/mL, about 160 mg/mL to about 180 mg/mL, or about 180 mg/mL to about 200 mg/mL. In certain embodiments, the concentration of furosemide in a liquid pharmaceutical formulation described herein can be about 50 mg/mL to about 250 mg/mL. In certain embodiments, the concentration of furosemide in a liquid pharmaceutical formulation described herein can be about 60 mg/mL to about 140 mg/mL. In certain embodiments, the concentration of furosemide in a liquid pharmaceutical formulation described herein can be about 80 mg/mL to about 120 mg/mL.

In certain embodiments, the concentration of furosemide in a liquid pharmaceutical formulation described herein can be about 50 mg/mL, about 60 mg/mL, about 70 mg/mL, about 80 mg/mL, about 90 mg/mL, about 100 mg/mL, about 120 mg/mL, about 140 mg/mL, about 160 mg/mL, about 180 mg/mL, about 200 mg/mL, about 220 mg/mL, or about 250 mg/mL. In certain embodiments, the concentration of furosemide in a liquid pharmaceutical formulation described herein is about 80 mg/mL. In certain embodiments, the concentration of furosemide in a liquid pharmaceutical formulation described herein is about 100 mg/mL. In certain embodiments, the concentration of furosemide in a liquid pharmaceutical formulation described herein is about 120 mg/mL.

In various embodiments, a liquid pharmaceutical formulation described herein may comprise a pharmaceutically acceptable salt of furosemide.

In various embodiments, a liquid pharmaceutical formulation described herein comprises a pharmaceutically acceptable salt of furosemide in an amount that provides about 40 mg to about 100 mg, about 45 mg to about 100 mg, about 50 mg to about 100 mg, about 55 mg to about 100 mg, about 60 mg to about 100 mg, about 65 mg to about 100 mg, about 70 mg to about 100 mg, about 75 mg to about 100 mg, about 80 mg to about 100 mg, about 85 mg to about 100 mg, about 90 mg to about 100 mg, about 95 mg to about 100 mg, about 40 mg to about 95 mg, about 40 mg to about 90 mg, about 40 mg to about 85 mg, about 40 mg to about 80 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 45 mg to about 95 mg, about 45 mg to about 90 mg, about 45 mg to about 85 mg, about 45 mg to about 80 mg, about 45 mg to about 75 mg, about 45 mg to about 70 mg, about 45 mg to about 65 mg, about 45 mg to about 60 mg, about 45 mg to about 55 mg, about 45 mg to about 50 mg, about 50 mg to about 95 mg, about 50 mg to about 90 mg, about 50 mg to about 85 mg, about 50 mg to about 80 mg, about 50 mg to about 75 mg, about 50 mg to about 70 mg, about 50 mg to about 65 mg, about 50 mg to about 60 mg, about 50 mg to about 55 mg, about 55 mg to about 95 mg, about 55 mg to about 90 mg, about 55 mg to about 85 mg, about 55 mg to about 80 mg, about 55 mg to about 75 mg, about 55 mg to about 70 mg, about 55 mg to about 65 mg, about 55 mg to about 60 mg, about 60 mg to about 95 mg, about 60 mg to about 90 mg, about 60 mg to about 85 mg, about 60 mg to about 80 mg, about 60 mg to about 75 mg, about 60 mg to about 70 mg, about 60 mg to about 65 mg, about 65 mg to about 95 mg, about 65 mg to about 90 mg, about 65 mg to about 85 mg, about 65 mg to about 80 mg, about 65 mg to about 75 mg, about 65 mg to about 70 mg, about 70 mg to about 95 mg, about 70 mg to about 90 mg, about 70 mg to about 85 mg, about 70 mg to about 80 mg, about 70 mg to about 75 mg, about 75 mg to about 95 mg, about 75 mg to about 90 mg, about 75 mg to about 85 mg, about 75 mg to about 80 mg, about 80 mg to about 95 mg, about 80 mg to about 90 mg, about 80 mg to about 85 mg, about 85 mg to about 95 mg, about 85 mg to about 90 mg, or about 90 mg to about 95 mg furosemide in its free acid form. In certain embodiments, a liquid pharmaceutical formulation described herein comprises a pharmaceutically acceptable salt of furosemide in an amount that provides about 40 mg to about 100 mg furosemide in its free acid form.

In certain embodiments, a liquid pharmaceutical formulation described herein comprises a pharmaceutically acceptable salt of furosemide in an amount that provides about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg furosemide in its free acid form. In certain embodiments, a liquid pharmaceutical formulation described herein comprises a pharmaceutically acceptable salt of furosemide in an amount that provides about 40 mg furosemide in its free acid form. In certain embodiments, a liquid pharmaceutical formulation described herein comprises a pharmaceutically acceptable salt of furosemide in an amount that provides about 80 mg furosemide in its free acid form.

In various embodiments, a liquid pharmaceutical formulation described herein comprises a pharmaceutically acceptable salt of furosemide in an amount that provides about 5% (w/w) to about 10% (w/w), about 6% (w/w) to about 10% (w/w), about 7% (w/w) to about 10% (w/w), about 8% (w/w) to about 10% (w/w), about 9% (w/w) to about 10% (w/w), about 5% (w/w) to about 9% (w/w), about 5% (w/w) to about 8% (w/w), about 5% (w/w) to about 7% (w/w), about 5% (w/w) to about 6% (w/w), about 6% (w/w) to about 9% (w/w), about 6% (w/w) to about 8% (w/w), about 6% (w/w) to about 7% (w/w), about 7% (w/w) to about 9% (w/w), about 7% (w/w) to about 8% (w/w), or about 8% (w/w) to about 9% (w/w) furosemide in its free acid form. In certain embodiments, a liquid pharmaceutical formulation described herein comprises a pharmaceutically acceptable salt of furosemide in an amount that provides about 5% (w/w) to about 10% (w/w) furosemide in its free acid form.

In certain embodiments, a liquid pharmaceutical formulation described herein comprises a pharmaceutically acceptable salt of furosemide in an amount that provides about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w) furosemide in its free acid form.

In certain embodiments, a liquid pharmaceutical formulation described herein comprises a pharmaceutically acceptable salt of furosemide in an amount that provides about 5% (w/w) furosemide. In certain embodiments, a liquid pharmaceutical formulation described herein comprises a pharmaceutically acceptable salt of furosemide in an amount that provides about 6% (w/w) furosemide in its free acid form. In certain embodiments, a liquid pharmaceutical formulation described herein comprises a pharmaceutically acceptable salt of furosemide in an amount that provides about 7% (w/w) furosemide in its free acid form. In certain embodiments, a liquid pharmaceutical formulation described herein comprises a pharmaceutically acceptable salt of furosemide in an amount that provides about 8% (w/w) furosemide in its free acid form. In certain embodiments, a liquid pharmaceutical formulation described herein comprises a pharmaceutically acceptable salt of furosemide in an amount that provides about 9% (w/w) furosemide in its free acid form. In certain embodiments, a liquid pharmaceutical formulation described herein comprises a pharmaceutically acceptable salt of furosemide in an amount that provides about 10% (w/w) furosemide in its free acid form.

In certain embodiments, a liquid pharmaceutical formulation described herein comprises a pharmaceutically acceptable salt of furosemide in an amount that provides a concentration of furosemide in its free acid form of about 50 mg/mL to about 250 mg/mL, about 60 mg/mL to about 250 mg/mL, about 70 mg/mL to about 250 mg/mL, about 80 mg/mL to about 250 mg/mL, about 90 mg/mL to about 250 mg/mL, about 100 mg/mL to about 250 mg/mL, about 120 mg/mL to about 250 mg/mL, about 140 mg/mL to about 250 mg/mL, about 160 mg/mL to about 250 mg/mL, about 180 mg/mL to about 250 mg/mL, about 200 mg/mL to about 250 mg/mL, about 50 mg/mL to about 200 mg/mL, about 50 mg/mL to about 180 mg/mL, about 50 mg/mL to about 160 mg/mL, about 50 mg/mL to about 140 mg/mL, about 50 mg/mL to about 120 mg/mL, about 50 mg/mL to about 100 mg/mL, about 50 mg/mL to about 90 mg/mL, about 50 mg/mL to about 80 mg/mL, about 50 mg/mL to about 70 mg/mL, about 50 mg/mL to about 60 mg/mL, about 60 mg/mL to about 200 mg/mL, about 60 mg/mL to about 180 mg/mL, about 60 mg/mL to about 160 mg/mL, about 60 mg/mL to about 140 mg/mL, about 60 mg/mL to about 120 mg/mL, about 60 mg/mL to about 100 mg/mL, about 60 mg/mL to about 90 mg/mL, about 60 mg/mL to about 80 mg/mL, about 60 mg/mL to about 70 mg/mL, about 70 mg/mL to about 200 mg/mL, about 70 mg/mL to about 180 mg/mL, about 70 mg/mL to about 160 mg/mL, about 70 mg/mL to about 140 mg/mL, about 70 mg/mL to about 120 mg/mL, about 70 mg/mL to about 100 mg/mL, about 70 mg/mL to about 90 mg/mL, about 70 mg/mL to about 80 mg/mL, about 80 mg/mL to about 200 mg/mL, about 80 mg/mL to about 180 mg/mL, about 80 mg/mL to about 160 mg/mL, about 80 mg/mL to about 140 mg/mL, about 80 mg/mL to about 120 mg/mL, about 80 mg/mL to about 100 mg/mL, about 80 mg/mL to about 90 mg/mL, about 90 mg/mL to about 200 mg/mL, about 90 mg/mL to about 180 mg/mL, about 90 mg/mL to about 160 mg/mL, about 90 mg/mL to about 140 mg/mL, about 90 mg/mL to about 120 mg/mL, about 90 mg/mL to about 100 mg/mL, about 100 mg/mL to about 200 mg/mL, about 100 mg/mL to about 180 mg/mL, about 100 mg/mL to about 160 mg/mL, about 100 mg/mL to about 140 mg/mL, about 100 mg/mL to about 120 mg/mL, about 120 mg/mL to about 200 mg/mL, about 120 mg/mL to about 180 mg/mL, about 120 mg/mL to about 160 mg/mL, about 120 mg/mL to about 140 mg/mL, about 140 mg/mL to about 200 mg/mL, about 140 mg/mL to about 180 mg/mL, about 140 mg/mL to about 160 mg/mL, about 160 mg/mL to about 200 mg/mL, about 160 mg/mL to about 180 mg/mL, or about 180 mg/mL to about 200 mg/mL. In certain embodiments, a liquid pharmaceutical formulation described herein comprises a pharmaceutically acceptable salt of furosemide in an amount that provides a concentration of furosemide in its free acid form of about 50 mg/mL to about 250 mg/mL. In certain embodiments, a liquid pharmaceutical formulation described herein comprises a pharmaceutically acceptable salt of furosemide in an amount that provides a concentration of furosemide in its free acid form of about 60 mg/mL to about 140 mg/mL. In certain embodiments, a liquid pharmaceutical formulation described herein comprises a pharmaceutically acceptable salt of furosemide in an amount that provides a concentration of furosemide in its free acid form of about 80 mg/mL to about 120 mg/mL.

In certain embodiments, a liquid pharmaceutical formulation described herein comprises a pharmaceutically acceptable salt of furosemide in an amount that provides a concentration of furosemide in its free acid form of about 40 mg/mL, about 50 mg/mL, about 60 mg/mL, about 70 mg/mL, about 80 mg/mL, about 90 mg/mL, about 100 mg/mL, about 120 mg/mL, about 140 mg/mL, about 160 mg/mL, about 180 mg/mL, about 200 mg/mL, about 220 mg/mL, or about 250 mg/mL. In certain embodiments, a liquid pharmaceutical formulation described herein comprises a pharmaceutically acceptable salt of furosemide in an amount that provides a concentration of furosemide in its free acid form of about 80 mg/mL. In certain embodiments, a liquid pharmaceutical formulation described herein comprises a pharmaceutically acceptable salt of furosemide in an amount that provides a concentration of furosemide in its free acid form of about 100 mg/mL. In certain embodiments, a liquid pharmaceutical formulation described herein comprises a pharmaceutically acceptable salt of furosemide in an amount that provides a concentration of furosemide in its free acid form of about 120 mg/mL.

### (ii) Torsemide

In various embodiments, the invention provides liquid pharmaceutical formulations comprising an effective amount of torsemide, or a pharmaceutically acceptable salt thereof, for the treatment of a condition described herein.

In certain embodiments, the effective amount of torsemide in a liquid pharmaceutical formulation described herein is about 20 mg to about 50 mg, about 25 mg to about 50 mg, about 30 mg to about 50 mg, about 35 mg to about 50 mg, about 40 mg to about 50 mg, about 45 mg to about 50 mg, about 20 mg to about 45 mg, about 20 mg to about 40 mg, about 20 mg to about 35 mg, about 20 mg to about 30 mg, about 20 mg to about 25 mg, about 25 mg to about 45 mg, about 25 mg to about 40 mg, about 25 mg to about 35 mg, about 25 mg to about 30 mg, about 30 mg to about 45 mg, about 30 mg to about 40 mg, about 30 mg to about 35 mg, about 35 mg to about 45 mg, about 35 mg to about 40 mg, or about 40 mg to about 45 mg. In certain embodiments, the effective amount of torsemide in a liquid pharmaceutical formulation described herein is about 20 mg to about 50 mg. In certain embodiments, the effective amount of torsemide in a liquid pharmaceutical formulation described herein is about 20 mg to about 40 mg.

In various embodiments, the effective amount of torsemide in a liquid pharmaceutical formulation described herein is about 20 mg, about 25 mg, about 30 mg, about 35 mg, about 40 mg, about 45 mg, or about 50 mg. In certain embodiments, the effective amount of torsemide in a liquid pharmaceutical formulation described herein is about 20 mg. In certain embodiments, the effective amount of torsemide in a liquid pharmaceutical formulation described herein is about 40 mg.

In certain embodiments, the concentration of torsemide in a liquid pharmaceutical formulation described herein can be about 25 mg/mL to about 125 mg/mL, about 30 mg/mL to about 125 mg/mL, about 35 mg/mL to about 125 mg/mL, about 40 mg/mL to about 125 mg/mL, about 45 mg/mL to about 125 mg/mL, about 50 mg/mL to about 125 mg/mL, about 60 mg/mL to about 125 mg/mL, about 70 mg/mL to about 125 mg/mL, about 80 mg/mL to about 125 mg/mL, about 90 mg/mL to about 125 mg/mL, about 100 mg/mL to about 125 mg/mL, about 25 mg/mL to about 100 mg/mL, about 25 mg/mL to about 90 mg/mL, about 25 mg/mL to about 80 mg/mL, about 25 mg/mL to about 70 mg/mL, about 25 mg/mL to about 60 mg/mL, about 25 mg/mL to about 50 mg/mL, about 25 mg/mL to about 45 mg/mL, about 25 mg/mL to about 40 mg/mL, about 25 mg/mL to about 35 mg/mL, about 25 mg/mL to about 30 mg/mL, about 30 mg/mL to about 100 mg/mL, about 30 mg/mL to about 90 mg/mL, about 30 mg/mL to about 80 mg/mL, about 30 mg/mL to about 70 mg/mL, about 30 mg/mL to about 60 mg/mL, about 30 mg/mL to about 50 mg/mL, about 30 mg/mL to about 45 mg/mL, about 30 mg/mL to about 40 mg/mL, about 30 mg/mL to about 35 mg/mL, about 35 mg/mL to about 100 mg/mL, about 35 mg/mL to about 90 mg/mL, about 35 mg/mL to about 80 mg/mL, about 35 mg/mL to about 70 mg/mL, about 35 mg/mL to about 60 mg/mL, about 35 mg/mL to about 50 mg/mL, about 35 mg/mL to about 45 mg/mL, about 35 mg/mL to about 40 mg/mL, about 40 mg/mL to about 100 mg/mL, about 40 mg/mL to about 90 mg/mL, about 40 mg/mL to about 80 mg/mL, about 40 mg/mL to about 70 mg/mL, about 40 mg/mL to about 60 mg/mL, about 40 mg/mL to about 50 mg/mL, about 40 mg/mL to about 45 mg/mL, about 45 mg/mL to about 100 mg/mL, about 45 mg/mL to about 90 mg/mL, about 45 mg/mL to about 80 mg/mL, about 45 mg/mL to about 70 mg/mL, about 45 mg/mL to about 60 mg/mL, about 45 mg/mL to about 50 mg/mL, about 50 mg/mL to about 100 mg/mL, about 50 mg/mL to about 90 mg/mL, about 50 mg/mL to about 80 mg/mL, about 50 mg/mL to about 70 mg/mL, about 50 mg/mL to about 60 mg/mL, about 60 mg/mL to about 100 mg/mL, about 60 mg/mL to about 90 mg/mL, about 60 mg/mL to about 80 mg/mL, about 60 mg/mL to about 70 mg/mL, about 70 mg/mL to about 100 mg/mL, about 70 mg/mL to about 90 mg/mL, about 70 mg/mL to about 80 mg/mL, about 80 mg/mL to about 100 mg/mL, about 80 mg/mL to about 90 mg/mL, or about 90 mg/mL to about 100 mg/mL. In certain embodiments, the concentration of torsemide in a liquid pharmaceutical formulation described herein can be about 25 mg/mL to about 125 mg/mL.

In certain embodiments, the concentration of torsemide in a liquid pharmaceutical formulation described herein can be about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 110 mg/mL, or about 125 mg/mL. In certain embodiments, the concentration of torsemide in a liquid pharmaceutical formulation described herein is about 40 mg/mL.

In various embodiments, a liquid pharmaceutical formulation described herein may comprise a pharmaceutically acceptable salt of torsemide.

In various embodiments, a liquid pharmaceutical formulation described herein comprises a pharmaceutically acceptable salt of torsemide in an amount that provides about 20 mg to about 50 mg, about 25 mg to about 50 mg, about 30 mg to about 50 mg, about 35 mg to about 50 mg, about 40 mg to about 50 mg, about 45 mg to about 50 mg, about 20 mg to about 45 mg, about 20 mg to about 40 mg, about 20 mg to about 35 mg, about 20 mg to about 30 mg, about 20 mg to about 25 mg, about 25 mg to about 45 mg, about 25 mg to about 40 mg, about 25 mg to about 35 mg, about 25 mg to about 30 mg, about 30 mg to about 45 mg, about 30 mg to about 40 mg, about 30 mg to about 35 mg, about 35 mg to about 45 mg, about 35 mg to about 40 mg, or about 40 mg to about 45 mg torsemide in its free acid form. In certain embodiments, a liquid pharmaceutical formulation described herein comprises a pharmaceutically acceptable salt of torsemide in an amount that provides about 20 mg to about 50 mg torsemide in its free acid form.

In certain embodiments, a liquid pharmaceutical formulation described herein comprises a pharmaceutically acceptable salt of torsemide in an amount that provides about 20 mg, about 25 mg, about 30 mg, about 35 mg, about 40 mg, about 45 mg, or about 50 mg torsemide in its free acid form. In certain embodiments, a liquid pharmaceutical formulation described herein comprises a pharmaceutically acceptable salt of torsemide in an amount that provides amount that provides about 20 mg torsemide in its free acid form. In certain embodiments, a liquid pharmaceutical formulation described herein comprises a pharmaceutically acceptable salt of torsemide in an amount that provides amount that provides about 40 mg torsemide in its free acid form.

In certain embodiments, a liquid pharmaceutical formulation described herein comprises a pharmaceutically acceptable salt of torsemide in an amount that provides a concentration of torsemide in its free acid form of about 25 mg/mL to about 125 mg/mL, about 30 mg/mL to about 125 mg/mL, about 35 mg/mL to about 125 mg/mL, about 40 mg/mL to about 125 mg/mL, about 45 mg/mL to about 125 mg/mL, about 50 mg/mL to about 125 mg/mL, about 60 mg/mL to about 125 mg/mL, about 70 mg/mL to about 125 mg/mL, about 80 mg/mL to about 125 mg/mL, about 90 mg/mL to about 125 mg/mL, about 100 mg/mL to about 125 mg/mL, about 25 mg/mL to about 100 mg/mL, about 25 mg/mL to about 90 mg/mL, about 25 mg/mL to about 80 mg/mL, about 25 mg/mL to about 70 mg/mL, about 25 mg/mL to about 60 mg/mL, about 25 mg/mL to about 50 mg/mL, about 25 mg/mL to about 45 mg/mL, about 25 mg/mL to about 40 mg/mL, about 25 mg/mL to about 35 mg/mL, about 25 mg/mL to about 30 mg/mL, about 30 mg/mL to about 100 mg/mL, about 30 mg/mL to about 90 mg/mL, about 30 mg/mL to about 80 mg/mL, about 30 mg/mL to about 70 mg/mL, about 30 mg/mL to about 60 mg/mL, about 30 mg/mL to about 50 mg/mL, about 30 mg/mL to about 45 mg/mL, about 30 mg/mL to about 40 mg/mL, about 30 mg/mL to about 35 mg/mL, about 35 mg/mL to about 100 mg/mL, about 35 mg/mL to about 90 mg/mL, about 35 mg/mL to about 80 mg/mL, about 35 mg/mL to about 70 mg/mL, about 35 mg/mL to about 60 mg/mL, about 35 mg/mL to about 50 mg/mL, about 35 mg/mL to about 45 mg/mL, about 35 mg/mL to about 40 mg/mL, about 40 mg/mL to about 100 mg/mL, about 40 mg/mL to about 90 mg/mL, about 40 mg/mL to about 80 mg/mL, about 40 mg/mL to about 70 mg/mL, about 40 mg/mL to about 60 mg/mL, about 40 mg/mL to about 50 mg/mL, about 40 mg/mL to about 45 mg/mL, about 45 mg/mL to about 100 mg/mL, about 45 mg/mL to about 90 mg/mL, about 45 mg/mL to about 80 mg/mL, about 45 mg/mL to about 70 mg/mL, about 45 mg/mL to about 60 mg/mL, about 45 mg/mL to about 50 mg/mL, about 50 mg/mL to about 100 mg/mL, about 50 mg/mL to about 90 mg/mL, about 50 mg/mL to about 80 mg/mL, about 50 mg/mL to about 70 mg/mL, about 50 mg/mL to about 60 mg/mL, about 60 mg/mL to about 100 mg/mL, about 60 mg/mL to about 90 mg/mL, about 60 mg/mL to about 80 mg/mL, about 60 mg/mL to about 70 mg/mL, about 70 mg/mL to about 100 mg/mL, about 70 mg/mL to about 90 mg/mL, about 70 mg/mL to about 80 mg/mL, about 80 mg/mL to about 100 mg/mL, about 80 mg/mL to about 90 mg/mL, or about 90 mg/mL to about 100 mg/mL. In certain embodiments, a liquid pharmaceutical formulation described herein comprises a pharmaceutically acceptable salt of torsemide in an amount that provides a concentration of torsemide in its free acid form of about 25 mg/mL to about 125 mg/mL.

In certain embodiments, a liquid pharmaceutical formulation described herein comprises a pharmaceutically acceptable salt of torsemide in an amount that provides a concentration of torsemide in its free acid form of about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 110 mg/mL, or about 125 mg/mL. In certain embodiments, a liquid pharmaceutical formulation described herein comprises a pharmaceutically acceptable salt of torsemide in an amount that provides a concentration of torsemide in its free acid form of about 40 mg/mL.

(iii) Bumetanide

In various embodiments, the invention provides liquid pharmaceutical formulations comprising an effective amount of bumetanide, or a pharmaceutically acceptable salt thereof, for the treatment of a condition described herein.

In certain embodiments, the effective amount of bumetanide in a liquid pharmaceutical formulation described herein is about 1 mg to about 2.5 mg, about 1.25 mg to about 2.5 mg, about 1.5 mg to about 2.5 mg, about 1.75 mg to about 2.5 mg, about 2 mg to about 2.5 mg, about 2.25 mg to about 50 mg, about 1 mg to about 2.25 mg, about 1 mg to about 2 mg, about 1 mg to about 1.75 mg, about 1 mg to about 1.5 mg, about 1 mg to about 1.25 mg, about 1.25 mg to about 2.25 mg, about 1.25 mg to about 2 mg, about 1.25 mg to about 1.75 mg, about 1.25 mg to about 1.5 mg, about 1.5 mg to about 2.25 mg, about 1.5 mg to about 2 mg, about 1.5 mg to about 1.75 mg, about 1.75 mg to about 2.25 mg, about 1.75 mg to about 2 mg, or about 2 mg to about 2.25 mg. In certain embodiments, the effective amount of bumetanide in a liquid pharmaceutical formulation described herein is about 1 mg to about 2.5 mg. In certain embodiments, the effective amount of bumetanide in a liquid pharmaceutical formulation described herein is about 1 mg to about 2 mg.

In various embodiments, the effective amount of bumetanide in a liquid pharmaceutical formulation described herein is about 1 mg, about 1.25 mg, about 1.5 mg, about 1.75 mg, about 2 mg, about 2.25 mg, or about 2.5 mg. In certain embodiments, the effective amount of bumetanide in a liquid pharmaceutical formulation described herein is about 1 mg. In certain embodiments, the effective amount of bumetanide in a liquid pharmaceutical formulation described herein is about 2 mg.

In certain embodiments, the concentration of bumetanide in a liquid pharmaceutical formulation described herein can be about 1.25 mg/mL to about 6.25 mg/mL, about 1.5 mg/mL to about 6.25 mg/mL, about 1.75 mg/mL to about 6.25 mg/mL, about 2 mg/mL to about 6.25 mg/mL, about 2.25 mg/mL to about 6.25 mg/mL, about 2.5 mg/mL to about 6.25 mg/mL, about 3 mg/mL to about 6.25 mg/mL, about 3.5 mg/mL to about 6.25 mg/mL, about 4 mg/mL to about 6.25 mg/mL, about 4.5 mg/mL to about 6.25 mg/mL, about 5 mg/mL to about 6.25 mg/mL, about 1.25 mg/mL to about 5 mg/mL, about 1.25 mg/mL to about 4.5 mg/mL, about 1.25 mg/mL to about 4 mg/mL, about 1.25 mg/mL to about 3.5 mg/mL, about 1.25 mg/mL to about 3 mg/mL, about 1.25 mg/mL to about 2.5 mg/mL, about 1.25 mg/mL to about 2.25 mg/mL, about 1.25 mg/mL to about 2 mg/mL, about 1.25 mg/mL to about 1.75 mg/mL, about 1.25 mg/mL to about 1.5 mg/mL, about 1.5 mg/mL to about 5 mg/mL, about 1.5 mg/mL to about 4.5 mg/mL, about 1.5 mg/mL to about 4 mg/mL, about 1.5 mg/mL to about 3.5 mg/mL, about 1.5 mg/mL to about 3 mg/mL, about 1.5 mg/mL to about 2.5 mg/mL, about 1.5 mg/mL to about 2.25 mg/mL, about 1.5 mg/mL to about 2 mg/mL, about 1.5 mg/mL to about 1.75 mg/mL, about 1.75 mg/mL to about 5 mg/mL, about 1.75 mg/mL to about 4.5 mg/mL, about 1.75 mg/mL to about 4 mg/mL, about 1.75 mg/mL to about 3.5 mg/mL, about 1.75 mg/mL to about 3 mg/mL, about 1.75 mg/mL to about 2.5 mg/mL, about 1.75 mg/mL to about 2.25 mg/mL, about 1.75 mg/mL to about 2 mg/mL, about 2 mg/mL to about 5 mg/mL, about 2 mg/mL to about 4.5 mg/mL, about 2 mg/mL to about 4 mg/mL, about 2 mg/mL to about 3.5 mg/mL, about 2 mg/mL to about 3 mg/mL, about 2 mg/mL to about 2.5 mg/mL, about 2 mg/mL to about 2.25 mg/mL, about 2.25 mg/mL to about 5 mg/mL, about 2.25 mg/mL to about 4.5 mg/mL, about 2.25 mg/mL to about 4 mg/mL, about 2.25 mg/mL to about 3.5 mg/mL, about 2.25 mg/mL to about 3 mg/mL, about 2.25 mg/mL to about 2.5 mg/mL, about 2.5 mg/mL to about 5 mg/mL, about 2.5 mg/mL to about 4.5 mg/mL, about 2.5 mg/mL to about 4 mg/mL, about 2.5 mg/mL to about 3.5 mg/mL, about 2.5 mg/mL to about 3 mg/mL, about 3 mg/mL to about 5 mg/mL, about 3 mg/mL to about 4.5 mg/mL, about 3 mg/mL to about 4 mg/mL, about 3 mg/mL to about 3.5 mg/mL, about 3.5 mg/mL to about 5 mg/mL, about 3.5 mg/mL to about 4.5 mg/mL, about 3.5 mg/mL to about 4 mg/mL, about 4 mg/mL to about 5 mg/mL, about 4 mg/mL to about 4.5 mg/mL, or about 4.5 mg/mL to about 5 mg/mL. In certain embodiments, the concentration of bumetanide in a liquid pharmaceutical formulation described herein can be about 1.25 mg/mL to about 6.25 mg/mL.

In certain embodiments, the concentration of bumetanide in a liquid pharmaceutical formulation described herein can be about 1 mg/mL, about 1.25 mg/mL, about 1.5 mg/mL, about 1.75 mg/mL, about 2 mg/mL, about 2.25 mg/mL, about 2.5 mg/mL, about 3 mg/mL, about 3.5 mg/mL, about 4 mg/mL, about 4.5 mg/mL, about 5 mg/mL, about 5.5 mg/mL, or about 6.25 mg/mL. In certain embodiments, the concentration of bumetanide in a liquid pharmaceutical formulation described herein is about 2 mg/mL.

In various embodiments, a liquid pharmaceutical formulation described herein may comprise a pharmaceutically acceptable salt of bumetanide.

In various embodiments, a liquid pharmaceutical formulation described herein comprises a pharmaceutically acceptable salt of bumetanide in an amount that provides about 1 mg to about 2.5 mg, about 1.25 mg to about 2.5 mg, about 1.5 mg to about 2.5 mg, about 1.75 mg to about 2.5 mg, about 2 mg to about 2.5 mg, about 2.25 mg to about 50 mg, about 1 mg to about 2.25 mg, about 1 mg to about 2 mg, about 1 mg to about 1.75 mg, about 1 mg to about 1.5 mg, about 1 mg to about 1.25 mg, about 1.25 mg to about 2.25 mg, about 1.25 mg to about 2 mg, about 1.25 mg to about 1.75 mg, about 1.25 mg to about 1.5 mg, about 1.5 mg to about 2.25 mg, about 1.5 mg to about 2 mg, about 1.5 mg to about 1.75 mg, about 1.75 mg to about 2.25 mg, about 1.75 mg to about 2 mg, or about 2 mg to about 2.25 mg bumetanide in its free acid form. In various embodiments, a liquid pharmaceutical formulation described herein comprises a pharmaceutically acceptable salt of bumetanide in an amount that provides about 1 mg to about 2.5 mg bumetanide in its free acid form.

In certain embodiments, a liquid pharmaceutical formulation described herein comprises a pharmaceutically acceptable salt of bumetanide in an amount that provides about 1 mg, about 1.25 mg, about 1.5 mg, about 1.75 mg, about 2 mg, about 2.25 mg, or about 2.5 mg bumetanide in its free acid form. In certain embodiments, a liquid pharmaceutical formulation described herein comprises a pharmaceutically acceptable salt of bumetanide in an amount that provides amount that provides about 1 mg bumetanide in its free acid form. In certain embodiments, a liquid pharmaceutical formulation described herein comprises a pharmaceutically acceptable salt of bumetanide in an amount that provides amount that provides about 2 mg bumetanide in its free acid form.

In certain embodiments, a liquid pharmaceutical formulation described herein comprises a pharmaceutically acceptable salt of bumetanide in an amount that provides a concentration of bumetanide in its free acid form of about 1.25 mg/mL to about 6.25 mg/mL, about 1.5 mg/mL to about 6.25 mg/mL, about 1.75 mg/mL to about 6.25 mg/mL, about 2 mg/mL to about 6.25 mg/mL, about 2.25 mg/mL to about 6.25 mg/mL, about 2.5 mg/mL to about 6.25 mg/mL, about 3 mg/mL to about 6.25 mg/mL, about 3.5 mg/mL to about 6.25 mg/mL, about 4 mg/mL to about 6.25 mg/mL, about 4.5 mg/mL to about 6.25 mg/mL, about 5 mg/mL to about 6.25 mg/mL, about 1.25 mg/mL to about 5 mg/mL, about 1.25 mg/mL to about 4.5 mg/mL, about 1.25 mg/mL to about 4 mg/mL, about 1.25 mg/mL to about 3.5 mg/mL, about 1.25 mg/mL to about 3 mg/mL, about 1.25 mg/mL to about 2.5 mg/mL, about 1.25 mg/mL to about 2.25 mg/mL, about 1.25 mg/mL to about 2 mg/mL, about 1.25 mg/mL to about 1.75 mg/mL, about 1.25 mg/mL to about 1.5 mg/mL, about 1.5 mg/mL to about 5 mg/mL, about 1.5 mg/mL to about 4.5 mg/mL, about 1.5 mg/mL to about 4 mg/mL, about 1.5 mg/mL to about 3.5 mg/mL, about 1.5 mg/mL to about 3 mg/mL, about 1.5 mg/mL to about 2.5 mg/mL, about 1.5 mg/mL to about 2.25 mg/mL, about 1.5 mg/mL to about 2 mg/mL, about 1.5 mg/mL to about 1.75 mg/mL, about 1.75 mg/mL to about 5 mg/mL, about 1.75 mg/mL to about 4.5 mg/mL, about 1.75 mg/mL to about 4 mg/mL, about 1.75 mg/mL to about 3.5 mg/mL, about 1.75 mg/mL to about 3 mg/mL, about 1.75 mg/mL to about 2.5 mg/mL, about 1.75 mg/mL to about 2.25 mg/mL, about 1.75 mg/mL to about 2 mg/mL, about 2 mg/mL to about 5 mg/mL, about 2 mg/mL to about 4.5 mg/mL, about 2 mg/mL to about 4 mg/mL, about 2 mg/mL to about 3.5 mg/mL, about 2 mg/mL to about 3 mg/mL, about 2 mg/mL to about 2.5 mg/mL, about 2 mg/mL to about 2.25 mg/mL, about 2.25 mg/mL to about 5 mg/mL, about 2.25 mg/mL to about 4.5 mg/mL, about 2.25 mg/mL to about 4 mg/mL, about 2.25 mg/mL to about 3.5 mg/mL, about 2.25 mg/mL to about 3 mg/mL, about 2.25 mg/mL to about 2.5 mg/mL, about 2.5 mg/mL to about 5 mg/mL, about 2.5 mg/mL to about 4.5 mg/mL, about 2.5 mg/mL to about 4 mg/mL, about 2.5 mg/mL to about 3.5 mg/mL, about 2.5 mg/mL to about 3 mg/mL, about 3 mg/mL to about 5 mg/mL, about 3 mg/mL to about 4.5 mg/mL, about 3 mg/mL to about 4 mg/mL, about 3 mg/mL to about 3.5 mg/mL, about 3.5 mg/mL to about 5 mg/mL, about 3.5 mg/mL to about 4.5 mg/mL, about 3.5 mg/mL to about 4 mg/mL, about 4 mg/mL to about 5 mg/mL, about 4 mg/mL to about 4.5 mg/mL, or about 4.5 mg/mL to about 5 mg/mL. In certain embodiments, a liquid pharmaceutical formulation described herein comprises a pharmaceutically acceptable salt of bumetanide in an amount that provides a concentration of bumetanide in its free acid form of about 1.25 mg/mL to about 6.25 mg/mL.

In certain embodiments, a liquid pharmaceutical formulation described herein comprises a pharmaceutically acceptable salt of bumetanide in an amount that provides a concentration of bumetanide in its free acid form of about 1 mg/mL, about 1.25 mg/mL, about 1.5 mg/mL, about 1.75 mg/mL, about 2 mg/mL, about 2.25 mg/mL, about 2.50 mg/mL, about 3 mg/mL, about 3.5 mg/mL, about 4 mg/mL, about 4.5 mg/mL, about 5 mg/mL, about 5.5 mg/mL, or about 6.25 mg/mL. In certain embodiments, a liquid pharmaceutical formulation described herein comprises a pharmaceutically acceptable salt of bumetanide in an amount that provides a concentration of bumetanide in its free acid form of about 2 mg/mL.

### (B) pH, Pharmaceutically Acceptable Buffers, and pH Adjusters

### (1) Pharmaceutically Acceptable Buffers

In various embodiments, a liquid pharmaceutical formulation described herein may further comprise a pharmaceutically acceptable buffer.

In various embodiments, a liquid pharmaceutical formulation described herein can comprise about 4 mg to about 16 mg, about 5 mg to about 16 mg, about 6 mg to about 16 mg, about 7 mg to about 16 mg, about 8 mg to about 16 mg, about 9 mg to about 16 mg, about 10 mg to about 16 mg, about 12 mg to about 16 mg, about 14 mg to about 16 mg, about 4 mg to about 14 mg, about 4 mg to about 12 mg, about 4 mg to about 10 mg, about 4 mg to about 9 mg, about 4 mg to about 8 mg, about 4 mg to about 7 mg, about 4 mg to about 6 mg, about 4 mg to about 5 mg, about 5 mg to about 14 mg, about 5 mg to about 12 mg, about 5 mg to about 10 mg, about 5 mg to about 9 mg, about 5 mg to about 8 mg, about 5 mg to about 7 mg, about 5 mg to about 6 mg, about 6 mg to about 14 mg, about 6 mg to about 12 mg, about 6 mg to about 10 mg, about 6 mg to about 9 mg, about 6 mg to about 8 mg, about 6 mg to about 7 mg, about 7 mg to about 14 mg, about 7 mg to about 12 mg, about 7 mg to about 10 mg, about 7 mg to about 9 mg, about 7 mg to about 8 mg, about 8 mg to about 14 mg, about 8 mg to about 12 mg, about 8 mg to about 10 mg, about 8 mg to about 9 mg, about 9 mg to about 14 mg, about 9 mg to about 12 mg, about 9 mg to about 10 mg, about 10 mg to about 14 mg, about 10 mg to about 12 mg, or about 12 mg to about 14 mg of the pharmaceutically acceptable buffer. In certain embodiments, a liquid pharmaceutical formulation described herein can comprise about 4 mg to about 16 mg of the pharmaceutically acceptable buffer.

In various embodiments, a liquid pharmaceutical formulation described herein can comprise about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, about 8 mg, about 8.1 mg, about 8.2 mg, about 8.3 mg, about 8.4 mg, about 8.5 mg, about 8.6 mg, about 8.7 mg, about 8.8 mg, about 8.9 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, about 10 mg, about 10.1 mg, about 10.2 mg, about 10.3 mg, about 10.4 mg, about 10.5 mg, about 10.6 mg, about 10.7 mg, about 10.8 mg, about 10.9 mg, about 11 mg, about 11.1 mg, about 11.2 mg, about 11.3 mg, about 11.4 mg, about 11.5 mg, about 11.6 mg, about 11.7 mg, about 11.8 mg, about 11.9 mg, about 12 mg, about 12.5 mg, about 13 mg, about 13.5 mg, about 14 mg, about 14.5 mg, about 15 mg, about 15.5 mg, or about 16 mg of the pharmaceutically acceptable buffer. In certain embodiments, a liquid pharmaceutical formulation described herein can comprise about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, or about 10 mg of the pharmaceutically acceptable buffer.

In various embodiments, a liquid pharmaceutical formulation described herein comprises about 0.1% (w/w) to about 1.5% (w/w), 0.3% (w/w) to about 1.5% (w/w), about 0.5% (w/w) to about 1.5% (w/w), about 0.7% (w/w) to about 1.5% (w/w), about 0.9% (w/w) to about 1.5% (w/w), about 1.1% (w/w) to about 1.5% (w/w), about 1.3% (w/w) to about 1.5% (w/w), about 0.1% (w/w) to about 1.3% (w/w), about 0.1% (w/w) to about 1.1% (w/w), about 0.1% (w/w) to about 0.9% (w/w), about 0.1% (w/w) to about 0.7% (w/w), about 0.1% (w/w) to about 0.5% (w/w), about 0.1% (w/w) to about 0.3% (w/w), about 0.3% (w/w) to about 1.3% (w/w), about 0.3% (w/w) to about 1.1% (w/w), about 0.3% (w/w) to about 0.9% (w/w), about 0.3% (w/w) to about 0.7% (w/w), about 0.3% (w/w) to about 0.5% (w/w), about 0.5% (w/w) to about 1.3% (w/w), about 0.5% (w/w) to about 1.1% (w/w), about 0.5% (w/w) to about 0.9% (w/w), about 0.5% (w/w) to about 0.7% (w/w), about 0.7% (w/w) to about 1.3% (w/w), about 0.7% (w/w) to about 1.1% (w/w), about 0.7% (w/w) to about 0.9% (w/w), about 0.9% (w/w) to about 1.3% (w/w), about 0.9% (w/w) to about 1.1% (w/w), or about 1.1% (w/w) to about 1.3% (w/w) of the pharmaceutically acceptable buffer. In various embodiments, a liquid pharmaceutical formulation described herein comprises about 0.1% (w/w) to about 1.5% (w/w) of the pharmaceutically acceptable buffer.

In various embodiments, a liquid pharmaceutical formulation described herein comprises about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.1% (w/w), about 1.2% (w/w), about 1.3% (w/w), about 1.4% (w/w), or about 1.5% (w/w) of the pharmaceutically acceptable buffer. In various embodiments, a liquid pharmaceutical formulation described herein comprises about 0.5% (w/w) of the pharmaceutically acceptable buffer. In various embodiments, a liquid pharmaceutical formulation described herein comprises about 0.6% (w/w) of the pharmaceutically acceptable buffer. In various embodiments, a liquid pharmaceutical formulation described herein comprises about 0.7% (w/w) of the pharmaceutically acceptable buffer. In various embodiments, a liquid pharmaceutical formulation described herein comprises about 0.8% (w/w) of the pharmaceutically acceptable buffer. In various embodiments, a liquid pharmaceutical formulation described herein comprises about 0.9% (w/w) of the pharmaceutically acceptable buffer.

In various embodiments, the concentration of the pharmaceutically acceptable buffer in a liquid pharmaceutical formulation described herein is about 50 mM to about 150 mM, about 60 mM to about 150 mM, about 70 mM to about 150 mM, about 80 mM to about 150 mM, about 90 mM to about 150 mM, about 100 mM to about 150 mM, about 110 mM to about 150 mM, about 120 mM to about 150 mM, about 130 mM to about 150 mM, about 140 mM to about 150 mM, about 50 mM to about 140 mM, about 50 mM to about 130 mM, about 50 mM to about 120 mM, about 50 mM to about 110 mM, about 50 mM to about 100 mM, about 50 mM to about 90 mM, about 50 mM to about 80 mM, about 50 mM to about 70 mM, about 50 mM to about 60 mM, about 60 mM to about 140 mM, about 60 mM to about 130 mM, about 60 mM to about 120 mM, about 60 mM to about 110 mM, about 60 mM to about 100 mM, about 60 mM to about 90 mM, about 60 mM to about 80 mM, about 60 mM to about 70 mM, about 70 mM to about 140 mM, about 70 mM to about 130 mM, about 70 mM to about 120 mM, about 70 mM to about 110 mM, about 70 mM to about 100 mM, about 70 mM to about 90 mM, about 70 mM to about 80 mM, about 80 mM to about 140 mM, about 80 mM to about 130 mM, about 80 mM to about 120 mM, about 80 mM to about 110 mM, about 80 mM to about 100 mM, about 80 mM to about 90 mM, about 90 mM to about 140 mM, about 90 mM to about 130 mM, about 90 mM to about 120 mM, about 90 mM to about 110 mM, about 90 mM to about 100 mM, about 100 mM to about 140 mM, about 100 mM to about 130 mM, about 100 mM to about 120 mM, about 100 mM to about 110 mM, about 110 mM to about 140 mM, about 110 mM to about 130 mM, about 110 mM to about 120 mM, about 120 mM to about 140 mM, about 130 mM to about 140 mM, or about 130 mM to about 140 mM.

In various embodiments, the concentration of the pharmaceutically acceptable buffer in a liquid pharmaceutical formulation described herein is about 50 mM, about 60 mM, about 70 mM, about 80 mM, about 90 mM, about 100 mM, about 110 mM, about 120 mM, about 130 mM, about 140 mM, or about 150 mM. In certain embodiments, the concentration of the pharmaceutically acceptable buffer in a liquid pharmaceutical formulation described herein is about 50 mM. In certain embodiments, the concentration of the pharmaceutically acceptable buffer in a liquid pharmaceutical formulation described herein is about 60 mM. In certain embodiments, the concentration of the pharmaceutically acceptable buffer in a liquid pharmaceutical formulation described herein is about 70 mM. In certain embodiments, the concentration of the pharmaceutically acceptable buffer in a liquid pharmaceutical formulation described herein is about 80 mM. In certain embodiments, the concentration of the pharmaceutically acceptable buffer in a liquid pharmaceutical formulation described herein is about 90 mM. In certain embodiments, the concentration of the pharmaceutically acceptable buffer in a liquid pharmaceutical formulation described herein is about 100 mM.

In various embodiments, the concentration of the pharmaceutically acceptable buffer in a liquid pharmaceutical formulation described herein is about 5 mg/mL to about 15 mg/mL, about 6 mg/mL to about 15 mg/mL, about 7 mg/mL to about 15 mg/mL, about 8 mg/mL to about 15 mg/mL, about 9 mg/mL to about 15 mg/mL, about 10 mg/mL to about 15 mg/mL, about 11 mg/mL to about 15 mg/mL, about 12 mg/mL to about 15 mg/mL, about 13 mg/mL to about 15 mg/mL, about 14 mg/mL to about 15 mg/mL, about 5 mg/mL to about 14 mg/mL, about 5 mg/mL to about 13 mg/mL, about 5 mg/mL to about 12 mg/mL, about 5 mg/mL to about 11 mg/mL, about 5 mg/mL to about 10 mg/mL, about 5 mg/mL to about 9 mg/mL, about 5 mg/mL to about 8 mg/mL, about 5 mg/mL to about 7 mg/mL, about 5 mg/mL to about 6 mg/mL, about 6 mg/mL to about 14 mg/mL, about 6 mg/mL to about 13 mg/mL, about 6 mg/mL to about 12 mg/mL, about 6 mg/mL to about 11 mg/mL, about 6 mg/mL to about 10 mg/mL, about 6 mg/mL to about 9 mg/mL, about 6 mg/mL to about 8 mg/mL, about 6 mg/mL to about 7 mg/mL, about 7 mg/mL to about 14 mg/mL, about 7 mg/mL to about 13 mg/mL, about 7 mg/mL to about 12 mg/mL, about 7 mg/mL to about 11 mg/mL, about 7 mg/mL to about 10 mg/mL, about 7 mg/mL to about 9 mg/mL, about 7 mg/mL to about 8 mg/mL, about 8 mg/mL to about 14 mg/mL, about 8 mg/mL to about 13 mg/mL, about 8 mg/mL to about 12 mg/mL, about 8 mg/mL to about 11 mg/mL, about 8 mg/mL to about 10 mg/mL, about 8 mg/mL to about 9 mg/mL, about 9 mg/mL to about 14 mg/mL, about 9 mg/mL to about 13 mg/mL, about 9 mg/mL to about 12 mg/mL, about 9 mg/mL to about 11 mg/mL, about 9 mg/mL to about 10 mg/mL, about 10 mg/mL to about 14 mg/mL, about 10 mg/mL to about 13 mg/mL, about 10 mg/mL to about 12 mg/mL, about 10 mg/mL to about 11 mg/mL, about 11 mg/mL to about 14 mg/mL, about 11 mg/mL to about 13 mg/mL, about 11 mg/mL to about 12 mg/mL, about 12 mg/mL to about 14 mg/mL, about 12 mg/mL to about 13 mg/mL, or about 13 mg/mL to about 14 mg/mL. In certain embodiments, the concentration of the pharmaceutically acceptable buffer in a liquid pharmaceutical formulation described herein is about 5 mg/mL to about 15 mg/mL.

In certain embodiments, the concentration of the pharmaceutically acceptable buffer in a liquid pharmaceutical formulation described herein is about 5 mg/mL, about 5.1 mg/mL, about 5.2 mg/mL, about 5.3 mg/mL, about 5.4 mg/mL, about 5.5 mg/mL, about 5.6 mg/mL, about 5.7 mg/mL, about 5.8 mg/mL, about 5.9 mg/mL, about 6 mg/mL, about 6.1 mg/mL, about 6.2 mg/mL, about 6.3 mg/mL, about 6.4 mg/mL, about 6.5 mg/mL, about 6.6 mg/mL, about 6.7 mg/mL, about 6.8 mg/mL, about 6.9 mg/mL, about 7 mg/mL, about 7.1 mg/mL, about 7.2 mg/mL, about 7.3 mg/mL, about 7.4 mg/mL, about 7.5 mg/mL, about 7.6 mg/mL, about 7.7 mg/mL, about 7.8 mg/mL, about 7.9 mg/mL, about 8 mg/mL, about 8.1 mg/mL, about 8.2 mg/mL, about 8.3 mg/mL, about 8.4 mg/mL, about 8.5 mg/mL, about 8.6 mg/mL, about 8.7 mg/mL, about 8.8 mg/mL, about 8.9 mg/mL, about 9 mg/mL, about 9.1 mg/mL, about 9.2 mg/mL, about 9.3 mg/mL, about 9.4 mg/mL, about 9.5 mg/mL, about 9.6 mg/mL, about 9.7 mg/mL, about 9.8 mg/mL, about 9.9 mg/mL, about 10 mg/mL, about 10.1 mg/mL, about 10.2 mg/mL, about 10.3 mg/mL, about 10.3 mg/mL, about 10.4 mg/mL, about 10.5 mg/mL, about 10.6 mg/mL, about 10.7 mg/mL, about 10.8 mg/mL, about 10.9 mg/mL, about 11 mg/mL, about 11.1 mg/mL, about 11.2 mg/mL, about 11.3 mg/mL, about 11.4 mg/mL, about 11.5 mg/mL, about 11.6 mg/mL, about 11.7 mg/mL, about 11.8 mg/mL, about 11.9 mg/mL, about 12 mg/mL, about 12.5 mg/mL, about 13 mg/mL, about 13.5 mg/mL, about 14 mg/mL, about 14.5 mg/mL, or about 15 mg/mL. In certain embodiments, the concentration of the pharmaceutically acceptable buffer in a liquid pharmaceutical formulation described herein is about 6 mg/mL. In certain embodiments, the concentration of the pharmaceutically acceptable buffer in a liquid pharmaceutical formulation described herein is about 7 mg/mL. In certain embodiments, the concentration of the pharmaceutically acceptable buffer in a liquid pharmaceutical formulation described herein is about 8 mg/mL. In certain embodiments, the concentration of the pharmaceutically acceptable buffer in a liquid pharmaceutical formulation described herein is about 9 mg/mL. In certain embodiments, the concentration of the pharmaceutically acceptable buffer in a liquid pharmaceutical formulation described herein is about 10 mg/mL. In certain embodiments, the concentration of the pharmaceutically acceptable buffer in a liquid pharmaceutical formulation described herein is about 11 mg/mL. In certain embodiments, the concentration of the pharmaceutically acceptable buffer in a liquid pharmaceutical formulation described herein is about 12 mg/mL.

In certain embodiments, the pharmaceutically acceptable buffer comprises a buffering agent selected from the group consisting of histidine, a citrate salt, sodium phosphate, potassium phosphate, tromethamine or a pharmaceutically acceptable salt thereof, and combinations thereof. In certain embodiments, the buffering agent is tromethamine, or a pharmaceutically acceptable salt thereof. In certain embodiments, the buffering agent is tromethamine.

In various embodiments, a liquid pharmaceutical formulation described herein can comprise about 4 mg to about 16 mg, about 5 mg to about 16 mg, about 6 mg to about 16 mg, about 7 mg to about 16 mg, about 8 mg to about 16 mg, about 9 mg to about 16 mg, about 10 mg to about 16 mg, about 12 mg to about 16 mg, about 14 mg to about 16 mg, about 4 mg to about 14 mg, about 4 mg to about 12 mg, about 4 mg to about 10 mg, about 4 mg to about 9 mg, about 4 mg to about 8 mg, about 4 mg to about 7 mg, about 4 mg to about 6 mg, about 4 mg to about 5 mg, about 5 mg to about 14 mg, about 5 mg to about 12 mg, about 5 mg to about 10 mg, about 5 mg to about 9 mg, about 5 mg to about 8 mg, about 5 mg to about 7 mg, about 5 mg to about 6 mg, about 6 mg to about 14 mg, about 6 mg to about 12 mg, about 6 mg to about 10 mg, about 6 mg to about 9 mg, about 6 mg to about 8 mg, about 6 mg to about 7 mg, about 7 mg to about 14 mg, about 7 mg to about 12 mg, about 7 mg to about 10 mg, about 7 mg to about 9 mg, about 7 mg to about 8 mg, about 8 mg to about 14 mg, about 8 mg to about 12 mg, about 8 mg to about mg, about 8 mg to about 9 mg, about 9 mg to about 14 mg, about 9 mg to about 12 mg, about 9 mg to about 10 mg, about 10 mg to about 14 mg, about 10 mg to about 12 mg, or about 12 mg to about 14 mg tromethamine. In certain embodiments, a liquid pharmaceutical formulation described herein can comprise about 4 mg to about 16 mg tromethamine.

In various embodiments, a liquid pharmaceutical formulation described herein can comprise about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, about 8 mg, about 8.1 mg, about 8.2 mg, about 8.3 mg, about 8.4 mg, about 8.5 mg, about 8.6 mg, about 8.7 mg, about 8.8 mg, about 8.9 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, about 10 mg, about 10.1 mg, about 10.2 mg, about 10.3 mg, about 10.4 mg, about 10.5 mg, about 10.6 mg, about 10.7 mg, about 10.8 mg, about 10.9 mg, about 11 mg, about 11.1 mg, about 11.2 mg, about 11.3 mg, about 11.4 mg, about 11.5 mg, about 11.6 mg, about 11.7 mg, about 11.8 mg, about 11.9 mg, about 12 mg, about 12.5 mg, about 13 mg, about 13.5 mg, about 14 mg, about 14.5 mg, about 15 mg, about 15.5 mg, or about 16 mg tromethamine. In certain embodiments, a liquid pharmaceutical formulation described herein comprises about 4.5 mg tromethamine. In certain embodiments, a liquid pharmaceutical formulation described herein comprises about 4.6 mg tromethamine. In certain embodiments, a liquid pharmaceutical formulation described herein comprises about 4.7 mg tromethamine. In certain embodiments, a liquid pharmaceutical formulation described herein comprises about 4.8 mg tromethamine. In certain embodiments, a liquid pharmaceutical formulation described herein comprises about 4.9 mg tromethamine. In certain embodiments, a liquid pharmaceutical formulation described herein comprises about 5 mg tromethamine. In certain embodiments, a liquid pharmaceutical formulation described herein comprises about 5.1 mg tromethamine. In certain embodiments, a liquid pharmaceutical formulation described herein comprises about 5.2 mg tromethamine. In certain embodiments, a liquid pharmaceutical formulation described herein comprises about 5.3 mg tromethamine. In certain embodiments, a liquid pharmaceutical formulation described herein comprises about 5.4 mg tromethamine. In certain embodiments, a liquid pharmaceutical formulation described herein comprises about 5.5 mg tromethamine. In certain embodiments, a liquid pharmaceutical formulation described herein comprises about 9 mg tromethamine. In certain embodiments, a liquid pharmaceutical formulation described herein comprises about 9.1 mg tromethamine. In certain embodiments, a liquid pharmaceutical formulation described herein comprises about 9.2 mg tromethamine. In certain embodiments, a liquid pharmaceutical formulation described herein comprises about 9.3 mg tromethamine. In certain embodiments, a liquid pharmaceutical formulation described herein comprises about 9.4 mg tromethamine. In certain embodiments, a liquid pharmaceutical formulation described herein comprises about 9.5 mg tromethamine. In certain embodiments, a liquid pharmaceutical formulation described herein comprises about 9.6 mg tromethamine. In certain embodiments, a liquid pharmaceutical formulation described herein comprises about 9.7 mg tromethamine. In certain embodiments, a liquid pharmaceutical formulation described herein comprises about 9.8 mg tromethamine. In certain embodiments, a liquid pharmaceutical formulation described herein comprises about 9.9 mg tromethamine. In certain embodiments, a liquid pharmaceutical formulation described herein comprises about 10 mg tromethamine.

In various embodiments, a liquid pharmaceutical formulation described herein comprises about 0.1% (w/w) to about 1.5% (w/w), 0.3% (w/w) to about 1.5% (w/w), about 0.5% (w/w) to about 1.5% (w/w), about 0.7% (w/w) to about 1.5% (w/w), about 0.9% (w/w) to about 1.5% (w/w), about 1.1% (w/w) to about 1.5% (w/w), about 1.3% (w/w) to about 1.5% (w/w), about 0.1% (w/w) to about 1.3% (w/w), about 0.1% (w/w) to about 1.1% (w/w), about 0.1% (w/w) to about 0.9% (w/w), about 0.1% (w/w) to about 0.7% (w/w), about 0.1% (w/w) to about 0.5% (w/w), about 0.1% (w/w) to about 0.3% (w/w), about 0.3% (w/w) to about 1.3% (w/w), about 0.3% (w/w) to about 1.1% (w/w), about 0.3% (w/w) to about 0.9% (w/w), about 0.3% (w/w) to about 0.7% (w/w), about 0.3% (w/w) to about 0.5% (w/w), about 0.5% (w/w) to about 1.3% (w/w), about 0.5% (w/w) to about 1.1% (w/w), about 0.5% (w/w) to about 0.9% (w/w), about 0.5% (w/w) to about 0.7% (w/w), about 0.7% (w/w) to about 1.3% (w/w), about 0.7% (w/w) to about 1.1% (w/w), about 0.7% (w/w) to about 0.9% (w/w), about 0.9% (w/w) to about 1.3% (w/w), about 0.9% (w/w) to about 1.1% (w/w), or about 1.1% (w/w) to about 1.3% (w/w) tromethamine. In various embodiments, a liquid pharmaceutical formulation described herein comprises about 0.1% (w/w) to about 1.5% (w/w) tromethamine.

In various embodiments, a liquid pharmaceutical formulation described herein comprises about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.1% (w/w), about 1.2% (w/w), about 1.3% (w/w), about 1.4% (w/w), or about 1.5% (w/w) tromethamine. In various embodiments, a liquid pharmaceutical formulation described herein comprises about 0.5% (w/w) tromethamine. In various embodiments, a liquid pharmaceutical formulation described herein comprises about 0.6% (w/w) tromethamine. In various embodiments, a liquid pharmaceutical formulation described herein comprises about 0.7% (w/w) tromethamine. In various embodiments, a liquid pharmaceutical formulation described herein comprises about 0.8% (w/w) tromethamine. In various embodiments, a liquid pharmaceutical formulation described herein comprises about 0.9% (w/w) tromethamine.

In various embodiments, the concentration of tromethamine in a liquid pharmaceutical formulation described herein is about 50 mM to about 150 mM, about 60 mM to about 150 mM, about 70 mM to about 150 mM, about 80 mM to about 150 mM, about 90 mM to about 150 mM, about 100 mM to about 150 mM, about 110 mM to about 150 mM, about 120 mM to about 150 mM, about 130 mM to about 150 mM, about 140 mM to about 150 mM, about 50 mM to about 140 mM, about 50 mM to about 130 mM, about 50 mM to about 120 mM, about 50 mM to about 110 mM, about 50 mM to about 100 mM, about 50 mM to about 90 mM, about 50 mM to about 80 mM, about 50 mM to about 70 mM, about 50 mM to about 60 mM, about 60 mM to about 140 mM, about 60 mM to about 130 mM, about 60 mM to about 120 mM, about 60 mM to about 110 mM, about 60 mM to about 100 mM, about 60 mM to about 90 mM, about 60 mM to about 80 mM, about 60 mM to about 70 mM, about 70 mM to about 140 mM, about 70 mM to about 130 mM, about 70 mM to about 120 mM, about 70 mM to about 110 mM, about 70 mM to about 100 mM, about 70 mM to about 90 mM, about 70 mM to about 80 mM, about 80 mM to about 140 mM, about 80 mM to about 130 mM, about 80 mM to about 120 mM, about 80 mM to about 110 mM, about 80 mM to about 100 mM, about 80 mM to about 90 mM, about 90 mM to about 140 mM, about 90 mM to about 130 mM, about 90 mM to about 120 mM, about 90 mM to about 110 mM, about 90 mM to about 100 mM, about 100 mM to about 140 mM, about 100 mM to about 130 mM, about 100 mM to about 120 mM, about 100 mM to about 110 mM, about 110 mM to about 140 mM, about 110 mM to about 130 mM, about 110 mM to about 120 mM, about 120 mM to about 140 mM, about 130 mM to about 140 mM, or about 130 mM to about 140 mM. In certain embodiments, the concentration of tromethamine in a liquid pharmaceutical formulation described herein is about 50 mM to about 150 mM.

In various embodiments, the concentration of tromethamine in a liquid pharmaceutical formulation described herein is about 50 mM, about 60 mM, about 70 mM, about 80 mM, about 90 mM, about 100 mM, about 110 mM, about 120 mM, about 130 mM, about 140 mM, or about 150 mM. In certain embodiments, the concentration of tromethamine in a liquid pharmaceutical formulation described herein is about 50 mM. In certain embodiments, the concentration of tromethamine in a liquid pharmaceutical formulation described herein is about 60 mM. In certain embodiments, the concentration of tromethamine in a liquid pharmaceutical formulation described herein is about 70 mM. In certain embodiments, the concentration of tromethamine in a liquid pharmaceutical formulation described herein is about 80 mM. In certain embodiments, the concentration of tromethamine in a liquid pharmaceutical formulation described herein is about 90 mM. In certain embodiments, the concentration of tromethamine in a liquid pharmaceutical formulation described herein is about 100 mM.

In various embodiments, the concentration of tromethamine in a liquid pharmaceutical formulation described herein is about 5 mg/mL to about 15 mg/mL, about 6 mg/mL to about 15 mg/mL, about 7 mg/mL to about 15 mg/mL, about 8 mg/mL to about 15 mg/mL, about 9 mg/mL to about 15 mg/mL, about 10 mg/mL to about 15 mg/mL, about 11 mg/mL to about 15 mg/mL, about 12 mg/mL to about 15 mg/mL, about 13 mg/mL to about 15 mg/mL, about 14 mg/mL to about 15 mg/mL, about 5 mg/mL to about 14 mg/mL, about 5 mg/mL to about 13 mg/mL, about 5 mg/mL to about 12 mg/mL, about 5 mg/mL to about 11 mg/mL, about 5 mg/mL to about 10 mg/mL, about 5 mg/mL to about 9 mg/mL, about 5 mg/mL to about 8 mg/mL, about 5 mg/mL to about 7 mg/mL, about 5 mg/mL to about 6 mg/mL, about 6 mg/mL to about 14 mg/mL, about 6 mg/mL to about 13 mg/mL, about 6 mg/mL to about 12 mg/mL, about 6 mg/mL to about 11 mg/mL, about 6 mg/mL to about 10 mg/mL, about 6 mg/mL to about 9 mg/mL, about 6 mg/mL to about 8 mg/mL, about 6 mg/mL to about 7 mg/mL, about 7 mg/mL to about 14 mg/mL, about 7 mg/mL to about 13 mg/mL, about 7 mg/mL to about 12 mg/mL, about 7 mg/mL to about 11 mg/mL, about 7 mg/mL to about 10 mg/mL, about 7 mg/mL to about 9 mg/mL, about 7 mg/mL to about 8 mg/mL, about 8 mg/mL to about 14 mg/mL, about 8 mg/mL to about 13 mg/mL, about 8 mg/mL to about 12 mg/mL, about 8 mg/mL to about 11 mg/mL, about 8 mg/mL to about 10 mg/mL, about 8 mg/mL to about 9 mg/mL, about 9 mg/mL to about 14 mg/mL, about 9 mg/mL to about 13 mg/mL, about 9 mg/mL to about 12 mg/mL, about 9 mg/mL to about 11 mg/mL, about 9 mg/mL to about 10 mg/mL, about 10 mg/mL to about 14 mg/mL, about 10 mg/mL to about 13 mg/mL, about 10 mg/mL to about 12 mg/mL, about 10 mg/mL to about 11 mg/mL, about 11 mg/mL to about 14 mg/mL, about 11 mg/mL to about 13 mg/mL, about 11 mg/mL to about 12 mg/mL, about 12 mg/mL to about 14 mg/mL, about 12 mg/mL to about 13 mg/mL, or about 13 mg/mL to about 14 mg/mL. In various embodiments, the concentration of tromethamine in a liquid pharmaceutical formulation described herein is about 5 mg/mL to about 15 mg/mL.

In certain embodiments, the concentration of tromethamine in a liquid pharmaceutical formulation described herein is about 5 mg/mL, about 5.1 mg/mL, about 5.2 mg/mL, about 5.3 mg/mL, about 5.4 mg/mL, about 5.5 mg/mL, about 5.6 mg/mL, about 5.7 mg/mL, about 5.8 mg/mL, about 5.9 mg/mL, about 6 mg/mL, about 6.1 mg/mL, about 6.2 mg/mL, about 6.3 mg/mL, about 6.4 mg/mL, about 6.5 mg/mL, about 6.6 mg/mL, about 6.7 mg/mL, about 6.8 mg/mL, about 6.9 mg/mL, about 7 mg/mL, about 7.1 mg/mL, about 7.2 mg/mL, about 7.3 mg/mL, about 7.4 mg/mL, about 7.5 mg/mL, about 7.6 mg/mL, about 7.7 mg/mL, about 7.8 mg/mL, about 7.9 mg/mL, about 8 mg/mL, about 8.1 mg/mL, about 8.2 mg/mL, about 8.3 mg/mL, about 8.4 mg/mL, about 8.5 mg/mL, about 8.6 mg/mL, about 8.7 mg/mL, about 8.8 mg/mL, about 8.9 mg/mL, about 9 mg/mL, about 9.1 mg/mL, about 9.2 mg/mL, about 9.3 mg/mL, about 9.4 mg/mL, about 9.5 mg/mL, about 9.6 mg/mL, about 9.7 mg/mL, about 9.8 mg/mL, about 9.9 mg/mL, about 10 mg/mL, about 10.1 mg/mL, about 10.2 mg/mL, about 10.3 mg/mL, about 10.3 mg/mL, about 10.4 mg/mL, about 10.5 mg/mL, about 10.6 mg/mL, about 10.7 mg/mL, about 10.8 mg/mL, about 10.9 mg/mL, about 11 mg/mL, about 11.1 mg/mL, about 11.2 mg/mL, about 11.3 mg/mL, about 11.4 mg/mL, about 11.5 mg/mL, about 11.6 mg/mL, about 11.7 mg/mL, about 11.8 mg/mL, about 11.9 mg/mL, about 12 mg/mL, about 12.5 mg/mL, about 13 mg/mL, about 13.5 mg/mL, about 14 mg/mL, about 14.5 mg/mL, or about 15 mg/mL. In certain embodiments, the concentration of tromethamine in a liquid pharmaceutical formulation described herein is about 6 mg/mL. In certain embodiments, the concentration of tromethamine in a liquid pharmaceutical formulation described herein is about 7 mg/mL. In certain embodiments, the concentration of tromethamine in a liquid pharmaceutical formulation described herein is about 8 mg/mL. In certain embodiments, the concentration of tromethamine in a liquid pharmaceutical formulation described herein is about 9 mg/mL. In certain embodiments, the concentration of tromethamine in a liquid pharmaceutical formulation described herein is about 10 mg/mL. In certain embodiments, the concentration of tromethamine in a liquid pharmaceutical formulation described herein is about 11 mg/mL. In certain embodiments, the concentration of tromethamine in a liquid pharmaceutical formulation described herein is about 12 mg/mL.

In various embodiments, the molar ratio of tromethamine to furosemide, or a pharmaceutically acceptable salt thereof, in a liquid pharmaceutical formulation described herein is about 1:1 to about 1:3.5, about 1:1.5 to about 1:3.5, about 1:2 to about 1:3.5, about 1:2.5 to about 1:3.5, about 1:3 to about 1:3.5, about 1:1 to about 1:3, about 1:1 to about 1:2.5, about 1:1 to about 1:2, about 1:1 to about 1:1.5, about 1:1.5 to about 1:3, about 1:1.5 to about 1:2.5, about 1:1.5 to about 1:2, about 1:2 to about 1:3, about 1:2 to about 1:2.5, or about 1:2.5 to about 1:3. In certain embodiments, the molar ratio of tromethamine to furosemide, or a pharmaceutically acceptable salt thereof, in a liquid pharmaceutical formulation described herein is about 1:1 to about 1:3.5. In certain embodiments, the molar ratio of tromethamine to furosemide, or a pharmaceutically acceptable salt thereof, in a liquid pharmaceutical formulation described herein is about 1:1.5 to about 1:3.5.

In various embodiments, the molar ratio of tromethamine to furosemide, or a pharmaceutically acceptable salt thereof, in a liquid pharmaceutical formulation described herein is about 1:1, about 1:1.5, about 1:2, about 1:2.5, about 1:3, or about 1:3.5. In certain embodiments, the molar ratio of tromethamine to furosemide, or a pharmaceutically acceptable salt thereof, in a liquid pharmaceutical formulation described herein is about 1:1. In certain embodiments, the molar ratio of tromethamine to furosemide, or a pharmaceutically acceptable salt thereof, in a liquid pharmaceutical formulation described herein is about 1:1.5. In certain embodiments, the molar ratio of tromethamine to furosemide, or a pharmaceutically acceptable salt thereof, in a liquid pharmaceutical formulation described herein is about 1:2. In certain embodiments, the molar ratio of tromethamine to furosemide, or a pharmaceutically acceptable salt thereof, in a liquid pharmaceutical formulation described herein is about 1:2.5. In certain embodiments, the molar ratio of tromethamine to furosemide, or a pharmaceutically acceptable salt thereof, in a liquid pharmaceutical formulation described herein is about 1:3. In certain embodiments, the molar ratio of tromethamine to furosemide, or a pharmaceutically acceptable salt thereof, in a liquid pharmaceutical formulation described herein is about 1:3.5.

### (2) pH Adjusters

In various embodiments, a liquid pharmaceutical formulation described herein further comprises a pharmaceutically acceptable pH adjuster.

Exemplary pharmaceutically acceptable pH adjusters include, but are not limited to, one or more of acetic acid, citric acid, fumaric acid, hydrochloric acid, malic acid, nitric acid, phosphoric acid, propionic acid, sulfuric acid, tartaric acid, ammonia solution, ammonium carbonate, diethanolamine, potassium hydroxide, sodium bicarbonate, sodium borate, sodium carbonate, sodium hydroxide, and trolamine.

In certain embodiments, the pharmaceutically acceptable pH adjuster is selected from the group consisting of potassium hydroxide, sodium hydroxide, hydrochloric acid, and combinations thereof. In certain embodiments, the pharmaceutically acceptable pH adjuster is hydrochloric acid and sodium hydroxide. In certain embodiments, the pharmaceutically acceptable pH adjuster is sodium hydroxide. In certain embodiments, the pharmaceutically acceptable pH adjuster is potassium hydroxide. In certain embodiments, the pharmaceutically acceptable pH adjuster is hydrochloric acid.

### (3) pH of a Liquid Pharmaceutical Formulation

In various embodiments, the pH of a liquid pharmaceutical formulation described herein can be from about 5.5 to about 8.5, from about 6 to about 8.5, from about 6.5 to about 8.5, from about 7 to about 8.5, from about 7.5 to about 8.5, from about 8 to about 8.5, from about 5.5 to about 8, from about 5.5 to about 7.5, from about 5.5 to about 7, from about 5.5 to about 6.5, from about 5.5 to about 6, from about 6 to about 8, from about 6 to about 7.5, from about 6 to about 7, from about 6 to about 6.5, from about 6.5 to about 8, from about 6.5 to about 7.5, from about 6.5 to about 7, from about 7 to about 8, from about 7 to about 7.5, or from about 7.5 to about 8. In certain embodiments, the pH of a liquid pharmaceutical formulation described herein is from about 6.5 to about 8.5. In certain embodiments, the pH of a liquid pharmaceutical formulation described herein is from about 7 to about 8. In certain embodiments, the pH of a liquid pharmaceutical formulation described herein is from about 7.5 to about 8.5.

In certain embodiments, the pH of a liquid pharmaceutical formulation described herein can be from about 7 to about 8.5, from about 7.1 to about 8.5, from about 7.3 to about 8.5, from about 7.5 to about 8.5, from about 7.7 to about 8.5, from about 7.9 to about 8.5, from about 8.1 to about 8.5, from about 8.3 to about 8.5, about 7 to about 8.3, about 7 to about 8.1, about 7 to about 7.9, about 7 to about 7.7, about 7 to about 7.5, about 7 to about 7.3, about 7 to about 7.1, about 7.1 to about 8.3, about 7.1 to about 8.1, about 7.1 to about 7.9, about 7.1 to about 7.7, about 7.1 to about 7.5, about 7.1 to about 7.3, about 7.3 to about 8.3, about 7.3 to about 8.1, about 7.3 to about 7.9, about 7.3 to about 7.7, about 7.3 to about 7.5, about 7.5 to about 8.3, about 7.5 to about 8.1, about 7.5 to about 7.9, about 7.5 to about 7.7, about 7.7 to about 8.3, about 7.7 to about 8.1, about 7.7 to about 7.9, about 7.9 to about 8.3, about 7.9 to about 8.1, or about 8.1 to about 8.3.

In certain embodiments, the pH of a liquid pharmaceutical formulation described herein can be about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, or about 8.5. In certain embodiments, the pH of a liquid pharmaceutical formulation described herein can be about 7.4. In certain embodiments, the pH of a liquid pharmaceutical formulation described herein can be about 7.5. In certain embodiments, the pH of a liquid pharmaceutical formulation described herein can be about 7.6. In certain embodiments, the pH of a liquid pharmaceutical formulation described herein can be about 7.7. In certain embodiments, the pH of a liquid pharmaceutical formulation described herein can be about 7.8. In certain embodiments, the pH of a liquid pharmaceutical formulation described herein can be about 7.9. In certain embodiments, the pH of a liquid pharmaceutical formulation described herein can be about 8.0.

In certain embodiments, the pH of a liquid pharmaceutical formulation described herein can be 5.5±0.3, 5.6=0.3, 5.7±0.3, 5.8=0.3, 5.9=0.3, 6=0.3, 6.1±0.3, 6.2±0.3, 6.3±0.3, 6.4±0.3, 6.5±0.3, 6.6±0.3, 6.7=0.3, 6.8=0.3, 6.9±0.3, 7±0.3, 7.1±0.3, 7.2=0.3, 7.3=0.3, 7.4±0.3, 7.5±0.3, 7.6±0.3, 7.7±0.3, 7.8±0.3, 7.9±0.3, 8=0.3, 8.1±0.3, 8.2±0.3, 8.3±0.3, 8.4±0.3, or 8.5±0.3. In certain embodiments, the pH of a liquid pharmaceutical formulation described herein can be 7.4±0.3. In certain embodiments, the pH of a liquid pharmaceutical formulation described herein can be 7.5±0.3. In certain embodiments, the pH of a liquid pharmaceutical formulation described herein can be 7.6±0.3. In certain embodiments, the pH of a liquid pharmaceutical formulation described herein can be 7.7±0.3. In certain embodiments, the pH of a liquid pharmaceutical formulation described herein can be 7.8±0.3. In certain embodiments, the pH of a liquid pharmaceutical formulation described herein can be 7.9±0.3. In certain embodiments, the pH of a liquid pharmaceutical formulation described herein can be 8.0±0.3.

### (C) Solubilizing Agents

In various embodiments, a liquid pharmaceutical formulation described herein may further comprise a solubilizing agent.

In various embodiments, a liquid pharmaceutical formulation described herein can comprise about 1 mg to about 50 mg, about 2 mg to about 50 mg, about 5 mg to about 50 mg, about 10 mg to about 50 mg, about 15 mg to about 50 mg, about 20 mg to about 50 mg, about 25 mg to about 50 mg, about 30 mg to about 50 mg, about 35 mg to about 50 mg, about 40 mg to about 50 mg, about 45 mg to about 50 mg, about 1 mg to about 45 mg, about 1 mg to about 40 mg, about 1 mg to about 35 mg, about 1 mg to about 30 mg, about 1 mg to about 25 mg, about 1 mg to about 20 mg, about 1 mg to about 15 mg, about 1 mg to about 10 mg, about 1 mg to about 5 mg, about 1 mg to about 2 mg, about 2 mg to about 45 mg, about 2 mg to about 40 mg, about 2 mg to about 35 mg, about 2 mg to about 30 mg, about 2 mg to about 25 mg, about 2 mg to about 20 mg, about 2 mg to about 15 mg, about 2 mg to about 10 mg, about 2 mg to about 5 mg, about 5 mg to about 45 mg, about 5 mg to about 40 mg, about 5 mg to about 35 mg, about 5 mg to about 30 mg, about 5 mg to about 25 mg, about 5 mg to about 20 mg, about 5 mg to about 15 mg, about 5 mg to about 10 mg, about 10 mg to about 45 mg, about 10 mg to about 40 mg, about 10 mg to about 35 mg, about 10 mg to about 30 mg, about 10 mg to about 25 mg, about 10 mg to about 20 mg, about 10 mg to about 15 mg, about 15 mg to about 45 mg, about 15 mg to about 40 mg, about 15 mg to about 35 mg, about 15 mg to about 30 mg, about 15 mg to about 25 mg, about 15 mg to about 20 mg, about 20 mg to about 45 mg, about 20 mg to about 40 mg, about 20 mg to about 35 mg, about 20 mg to about 30 mg, about 20 mg to about 25 mg, about 25 mg to about 45 mg, about 25 mg to about 40 mg, about 25 mg to about 35 mg, about 25 mg to about 30 mg, about 30 mg to about 45 mg, about 30 mg to about 40 mg, about 30 mg to about 35 mg, about 35 mg to about 45 mg, about 35 mg to about 40 mg, or about 40 mg to about 45 mg of the solubilizing agent. In certain embodiments, a liquid pharmaceutical formulation described herein can comprise about 1 mg to about 50 mg of the solubilizing agent. In certain embodiments, a liquid pharmaceutical formulation described herein can comprise about 15 mg to about 50 mg of the solubilizing agent.

In various embodiments, a liquid pharmaceutical formulation described herein can comprise about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg of the solubilizing agent. In certain embodiments, a liquid pharmaceutical formulation described herein can comprise about 1 mg of the solubilizing agent. In certain embodiments, a liquid pharmaceutical formulation described herein can comprise about 2 mg of the solubilizing agent. In certain embodiments, a liquid pharmaceutical formulation described herein can comprise about 5 mg of the solubilizing agent. In certain embodiments, a liquid pharmaceutical formulation described herein can comprise about 10 mg of the solubilizing agent. In certain embodiments, a liquid pharmaceutical formulation described herein can comprise about 15 mg of the solubilizing agent. In certain embodiments, a liquid pharmaceutical formulation described herein can comprise about 20 mg of the solubilizing agent. In certain embodiments, a liquid pharmaceutical formulation described herein can comprise about 25 mg of the solubilizing agent. In certain embodiments, a liquid pharmaceutical formulation described herein can comprise about 30 mg of the solubilizing agent. In certain embodiments, a liquid pharmaceutical formulation described herein can comprise about 35 mg of the solubilizing agent. In certain embodiments, a liquid pharmaceutical formulation described herein can comprise about 40 mg of the solubilizing agent. In certain embodiments, a liquid pharmaceutical formulation described herein can comprise about 45 mg of the solubilizing agent. In certain embodiments, a liquid pharmaceutical formulation described herein can comprise about 50 mg of the solubilizing agent.

In various embodiments, a liquid pharmaceutical formulation described herein can comprise about 1% (w/w) to about 5% (w/w), about 2% (w/w) to about 5% (w/w), about 3% (w/w) to about 5% (w/w), about 4% (w/w) to about 5% (w/w), about 1% (w/w) to about 4% (w/w), about 1% (w/w) to about 3% (w/w), about 1% (w/w) to about 2% (w/w), about 2% (w/w) to about 4% (w/w), about 2% (w/w) to about 3% (w/w), or about 3% (w/w) to about 4% (w/w) of the solubilizing agent. In various embodiments, a liquid pharmaceutical formulation described herein can comprise about 1% (w/w) to about 5% (w/w) of the solubilizing agent.

In various embodiments, a liquid pharmaceutical formulation described herein can comprise about 1% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), about 2% (w/w), about 2.2% (w/w), about 2.4% (w/w), about 2.6% (w/w), about 2.8% (w/w), about 3% (w/w), about 3.5% (w/w), about 4% (w/w), about 4.5% (w/w), or about 5% (w/w) of the solubilizing agent. In certain embodiments, a liquid pharmaceutical formulation described herein can comprise about 1% (w/w) of the solubilizing agent. In certain embodiments, a liquid pharmaceutical formulation described herein can comprise about 2% (w/w) of the solubilizing agent. In certain embodiments, a liquid pharmaceutical formulation described herein can comprise about 3% (w/w) of the solubilizing agent. In certain embodiments, a liquid pharmaceutical formulation described herein can comprise about 4% (w/w) of the solubilizing agent. In certain embodiments, a liquid pharmaceutical formulation described herein can comprise about 5% (w/w) of the solubilizing agent.

In various embodiments, the concentration of the solubilizing agent in a liquid pharmaceutical formulation described herein is about 15 mg/mL to about 50 mg/mL, about 20 mg/mL to about 50 mg/mL, about 25 mg/mL to about 50 mg/mL, about 30 mg/mL to about 50 mg/mL, about 35 mg/mL to about 50 mg/mL, about 40 mg/mL to about 50 mg/mL, about 45 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 45 mg/mL, about 25 mg/mL to about 40 mg/mL, about 25 mg/mL to about 35 mg/mL, about 25 mg/mL to about 30 mg/mL, about 30 mg/mL to about 45 mg/mL, about 30 mg/mL to about 40 mg/mL, about 30 mg/mL to about 35 mg/mL, about 35 mg/mL to about 45 mg/mL, about 35 mg/mL to about 40 mg/mL, or about 40 mg/mL to about 45 mg/mL.

In various embodiments, the concentration of the solubilizing agent in a liquid pharmaceutical formulation described herein is about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL. In certain embodiments, the concentration of the solubilizing agent in a liquid pharmaceutical formulation described herein is about 15 mg/mL. In certain embodiments, the concentration of the solubilizing agent in a liquid pharmaceutical formulation described herein is about 20 mg/mL. In certain embodiments, the concentration of the solubilizing agent in a liquid pharmaceutical formulation described herein is about 25 mg/mL. In certain embodiments, the concentration of the solubilizing agent in a liquid pharmaceutical formulation described herein is about 30 mg/mL. In certain embodiments, the concentration of the solubilizing agent in a liquid pharmaceutical formulation described herein is about 35 mg/mL. In certain embodiments, the concentration of the solubilizing agent in a liquid pharmaceutical formulation described herein is about 40 mg/mL. In certain embodiments, the concentration of the solubilizing agent in a liquid pharmaceutical formulation described herein is about 45 mg/mL. In certain embodiments, the concentration of the solubilizing agent in a liquid pharmaceutical formulation described herein is about 50 mg/mL.

Examples of solubilizing agents suitable for use in the liquid pharmaceutical formulations described herein include, but are not limited to, benzyl alcohol, a polysorbate (e.g., polysorbate 20 (e.g., Tween® 20) or polysorbate 80 (e.g., Tween® 80), sodium stearate, 4-(5-dodecyl)benzenesulfonate, docusate (dioctyl sodium sulfosuccinate), alkyl ether phosphates, sodium lauryl sulfate, polyethylene glycol ethers, polyoxyethylene 15 hydroxy stearate (e.g., Macrogol 15 hydroxy stearate, Solutol HS158), polyoxyethylene castor oil derivatives (e.g., Cremophor® EL, ELP, RH 40), polyoxyethylene stearates (e.g., Myrj®), sorbitan fatty acid esters (e.g., Span®), polyoxyethylene alkyl ethers (e.g., Brij®), and polyoxyethylene nonylphenol ether (e.g., Nonoxynol®).

In certain embodiments, the solubilizing agent is benzyl alcohol. In certain embodiments, the solubilizing agent is polysorbate 20. In certain embodiments, the solubilizing agent is polysorbate 80. In some embodiments, the solubilizing agent is a combination thereof.

In various embodiments, a liquid pharmaceutical formulation described herein can comprise about 15 mg to about 50 mg, about 20 mg to about 50 mg, about 25 mg to about 50 mg, about 30 mg to about 50 mg, about 35 mg to about 50 mg, about 40 mg to about 50 mg, about 45 mg to about 50 mg, about 15 mg to about 45 mg, about 15 mg to about 40 mg, about 15 mg to about 35 mg, about 15 mg to about 30 mg, about 15 mg to about 25 mg, about 15 mg to about 20 mg, about 20 mg to about 45 mg, about 20 mg to about 40 mg, about 20 mg to about 35 mg, about 20 mg to about 30 mg, about 20 mg to about 25 mg, about 25 mg to about 45 mg, about 25 mg to about 40 mg, about 25 mg to about 35 mg, about 25 mg to about 30 mg, about 30 mg to about 45 mg, about 30 mg to about 40 mg, about 30 mg to about 35 mg, about 35 mg to about 45 mg, about 35 mg to about 40 mg, or about 40 mg to about 45 mg benzyl alcohol. In various embodiments, a liquid pharmaceutical formulation described herein can comprise about 15 mg to about 50 mg benzyl alcohol.

In various embodiments, a liquid pharmaceutical formulation described herein can comprise about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg benzyl alcohol. In certain embodiments, a liquid pharmaceutical formulation described herein can comprise about 15 mg benzyl alcohol. In certain embodiments, a liquid pharmaceutical formulation described herein can comprise about 20 mg benzyl alcohol. In certain embodiments, a liquid pharmaceutical formulation described herein can comprise about 25 mg benzyl alcohol. In certain embodiments, a liquid pharmaceutical formulation described herein can comprise about 30 mg benzyl alcohol. In certain embodiments, a liquid pharmaceutical formulation described herein can comprise about 35 mg benzyl alcohol. In certain embodiments, a liquid pharmaceutical formulation described herein can comprise about 40 mg benzyl alcohol. In certain embodiments, a liquid pharmaceutical formulation described herein can comprise about 45 mg benzyl alcohol. In certain embodiments, a liquid pharmaceutical formulation described herein can comprise about 50 mg benzyl alcohol. In various embodiments, a liquid pharmaceutical formulation described herein can comprise about 1% (w/w) to about 5% (w/w), about 2% (w/w) to about 5% (w/w), about 3% (w/w) to about 5% (w/w), about 4% (w/w) to about 5% (w/w), about 1% (w/w) to about 4% (w/w), about 1% (w/w) to about 3% (w/w), about 1% (w/w) to about 2% (w/w), about 2% (w/w) to about 4% (w/w), about 2% (w/w) to about 3% (w/w), or about 3% (w/w) to about 4% (w/w) benzyl alcohol.

In various embodiments, a liquid pharmaceutical formulation described herein can comprise about 1% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), about 2% (w/w), about 2.2% (w/w), about 2.4% (w/w), about 2.6% (w/w), about 2.8% (w/w), about 3% (w/w), about 3.5% (w/w), about 4% (w/w), about 4.5% (w/w), or about 5% (w/w) benzyl alcohol. In certain embodiments, a liquid pharmaceutical formulation described herein can comprise about 1% (w/w) benzyl alcohol. In certain embodiments, a liquid pharmaceutical formulation described herein can comprise about 2% (w/w) benzyl alcohol. In certain embodiments, a liquid pharmaceutical formulation described herein can comprise about 3% (w/w) benzyl alcohol. In certain embodiments, a liquid pharmaceutical formulation described herein can comprise about 4% (w/w) benzyl alcohol. In certain embodiments, a liquid pharmaceutical formulation described herein can comprise about 5% (w/w) benzyl alcohol.

In various embodiments, the concentration of benzyl alcohol in a liquid pharmaceutical formulation described herein is about 15 mg/mL to about 50 mg/mL, about 20 mg/mL to about 50 mg/mL, about 25 mg/mL to about 50 mg/mL, about 30 mg/mL to about 50 mg/mL, about 35 mg/mL to about 50 mg/mL, about 40 mg/mL to about 50 mg/mL, about 45 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 45 mg/mL, about 25 mg/mL to about 40 mg/mL, about 25 mg/mL to about 35 mg/mL, about 25 mg/mL to about 30 mg/mL, about 30 mg/mL to about 45 mg/mL, about 30 mg/mL to about 40 mg/mL, about 30 mg/mL to about 35 mg/mL, about 35 mg/mL to about 45 mg/mL, about 35 mg/mL to about 40 mg/mL, or about 40 mg/mL to about 45 mg/mL.

In various embodiments, the concentration of benzyl alcohol in a liquid pharmaceutical formulation described herein is about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL. In certain embodiments, the concentration of benzyl alcohol in a liquid pharmaceutical formulation described herein is about 15 mg/mL. In certain embodiments, the concentration of benzyl alcohol in a liquid pharmaceutical formulation described herein is about 20 mg/mL. In certain embodiments, the concentration of benzyl alcohol in a liquid pharmaceutical formulation described herein is about 25 mg/mL. In certain embodiments, the concentration of benzyl alcohol in a liquid pharmaceutical formulation described herein is about 30 mg/mL. In certain embodiments, the concentration of benzyl alcohol in a liquid pharmaceutical formulation described herein is about 35 mg/mL. In certain embodiments, the concentration of benzyl alcohol in a liquid pharmaceutical formulation described herein is about 40 mg/mL. In certain embodiments, the concentration of benzyl alcohol in a liquid pharmaceutical formulation described herein is about 45 mg/mL. In certain embodiments, the concentration of benzyl alcohol in a liquid pharmaceutical formulation described herein is about 50 mg/mL.

In various embodiments, the molar ratio of tromethamine to benzyl alcohol in a liquid pharmaceutical formulation described herein is about 2:1 to about 1:7, about 1.5:1 to about 1:7, about 1:1.5 to about 1:7, about 1:2 to about 1:7, about 1:2.5 to about 1:7, about 1:3 to about 1:7, about 1:3.5 to about 1:7, about 1:4 to about 1:7, about 1:4.5 to about 1:7, about 1:5 to about 1:7, about 1:5.5 to about 1:7, about 1:6 to about 1:7, about 1:6.5 to about 1:7, about 1:2 to about 1:7, about 2:1 to about 1:6.5, about 2:1 to about 1:6, about 2:1 to about 1:5.5, about 2:1 to about 1:5, about 2:1 to about 1:4.5, about 2:1 to about 1:4, about 2:1 to about 1:3.5, about 2:1 to about 1:3, about 2:1 to about 1:2.5, about 2:1 to about 1:2, about 2:1 to about 1:1.5, about 2:1 to about 1:1, about 2:1 to about 1.5:1, about 1.5:1 to about 1:65, about 1.5:1 to about 1:6, about 1.5:1 to about 1:5.5, about 1.5:1 to about 1:5, about 1.5:1 to about 1:4.5, about 1.5:1 to about 1:4, about 1.5:1 to about 1:3.5, about 1.5:1 to about 1:3, about 1.5:1 to about 1:2.5, about 1.5:1 to about 1:2, about 1.5:1 to about 1:1.5, about 1.5:1 to about 1:1, about 1:1 to about 1:65, about 1:1 to about 1:6, about 1:1 to about 1:5.5, about 1:1 to about 1:5, about 1:1 to about 1:4.5, about 1:1 to about 1:4, about 1:1 to about 1:3.5, about 1:1 to about 1:3, about 1:1 to about 1:2.5, about 1:1 to about 1:2, about 1:1 to about 1:1.5, about 1:1.5 to about 1:65, about 1:1.5 to about 1:6, about 1:1.5 to about 1:5.5, about 1:1.5 to about 1:5, about 1:1.5 to about 1:4.5, about 1:1.5 to about 1:4, about 1:1.5 to about 1:3.5, about 1:1.5 to about 1:3, about 1:1.5 to about 1:2.5, about 1:1.5 to about 1:2, about 1:2 to about 1:65, about 1:2 to about 1:6, about 1:2 to about 1:5.5, about 1:2 to about 1:5, about 1:2 to about 1:4.5, about 1:2 to about 1:4, about 1:2 to about 1:3.5, about 1:2 to about 1:3, about 1:2 to about 1:2.5, about 1:2.5 to about 1:65, about 1:2.5 to about 1:6, about 1:2.5 to about 1:5.5, about 1:2.5 to about 1:5, about 1:2.5 to about 1:4.5, about 1:2.5 to about 1:4, about 1:2.5 to about 1:3.5, about 1:2.5 to about 1:3, about 1:3 to about 1:6, about 1:3 to about 1:5.5, about 1:3 to about 1:5, about 1:3 to about 1:4.5, about 1:3 to about 1:4, about 1:3 to about 1:3.5, about 1:3.5 to about 1:65, about 1:3.5 to about 1:6, about 1:3.5 to about 1:5.5, about 1:3.5 to about 1:5, about 1:3.5 to about 1:4.5, about 1:3.5 to about 1:4, about 1:4 to about 1:65, about 1:4 to about 1:6, about 1:4 to about 1:5.5, about 1:4 to about 1:5, about 1:4 to about 1:4.5, about 1:4.5 to about 1:65, about 1:4.5 to about 1:6, about 1:4.5 to about 1:5.5, about 1:4.5 to about 1:5, about 1:5 to about 1:65, about 1:5 to about 1:6, about 1:5 to about 1:5.5, about 1:5.5 to about 1:65, about 1:5.5 to about 1:6 or about 1:6 to about 1:65. In certain embodiments, the molar ratio of tromethamine to benzyl alcohol in a liquid pharmaceutical formulation described herein is about 1.5:1 to about 1:6.5.

In various embodiments, the molar ratio of tromethamine to benzyl alcohol in a liquid pharmaceutical formulation described herein is about 2:1, about 1.5:1, about 1:1, about 1:1.5, about 1:2, about 1:2.5, about 1:3, about 1:3.5, about 1:4, about 1:4.5, about 1:5, about 1:5.5, about 1:6, about 1:6.5, about 1:7. In certain embodiments, the molar ratio of tromethamine to benzyl alcohol in a liquid pharmaceutical formulation described herein is about 1.5:1. In certain embodiments, the molar ratio of tromethamine to benzyl alcohol in a liquid pharmaceutical formulation described herein is about 1:2. In certain embodiments, the molar ratio of tromethamine to benzyl alcohol in a liquid pharmaceutical formulation described herein is about 1:4. In certain embodiments, the molar ratio of tromethamine to benzyl alcohol in a liquid pharmaceutical formulation described herein is about 1:6.5.

### (D) Additional Pharmaceutically Acceptable Excipients

### (1) Osmolarity and Osmolality

In various embodiments, a liquid pharmaceutical formulation described herein can further comprise an osmolarity or an osmolality adjuster.

In various embodiments, the osmolarity or the osmolality adjuster is selected from the group comprising sodium chloride, potassium chloride, isosorbide, mannitol, xylitol, and combinations thereof. In certain embodiments, the osmolarity or the osmolality adjuster is sodium chloride, potassium chloride, or a combination thereof. In certain embodiments, the osmolarity or the osmolality adjuster is sodium chloride. In certain embodiments, the osmolarity or the osmolality adjuster is potassium chloride.

In various embodiments, a liquid pharmaceutical formulation described herein has an osmolarity in the range of from about 100 mOsm/kg to about 1600 mOsm/kg, from about 200 mOsm/kg to about 1600 mOsm/kg, from about 300 mOsm/kg to about 1600 mOsm/kg, from about 400 mOsm/kg to about 1600 mOsm/kg, from about 600 mOsm/kg to about 1600 mOsm/kg, from about 800 mOsm/kg to about 1600 mOsm/kg, from about 1200 mOsm/kg to about 1600 mOsm/kg, from about 100 mOsm/kg to about 1200 mOsm/kg, from about 100 mOsm/kg to about 800 mOsm/kg, from about 100 mOsm/kg to about 600 mOsm/kg, from about 100 mOsm/kg to about 400 mOsm/kg, from about 100 mOsm/kg to about 300 mOsm/kg, from about 100 mOsm/kg to about 200 mOsm/kg, from about 200 mOsm/kg to about 1200 mOsm/kg, from about 200 mOsm/kg to about 800 mOsm/kg, from about 200 mOsm/kg to about 600 mOsm/kg, from about 200 mOsm/kg to about 400 mOsm/kg, from about 200 mOsm/kg to about 300 mOsm/kg, from about 300 mOsm/kg to about 1200 mOsm/kg, from about 300 mOsm/kg to about 800 mOsm/kg, from about 300 mOsm/kg to about 600 mOsm/kg, from about 300 mOsm/kg to about 400 mOsm/kg, from about 400 mOsm/kg to about 1200 mOsm/kg, from about 400 mOsm/kg to about 800 mOsm/kg, from about 400 mOsm/kg to about 600 mOsm/kg, from about 600 mOsm/kg to about 1200 mOsm/kg, from about 600 mOsm/kg to about 800 mOsm/kg, or from about 800 mOsm/kg to about 1200 mOsm/kg. In certain embodiments, a liquid pharmaceutical formulation described herein has an osmolarity in the range of from about 200 mOsm/kg to about 400 mOsm/kg. In certain embodiments, a liquid pharmaceutical formulation described herein has an osmolarity in the range of from about 300 mOsm/kg to about 600 mOsm/kg. In certain embodiments, a liquid pharmaceutical formulation described herein has an osmolarity in the range of from about 300 mOsm/kg to about 450 mOsm/kg.

In certain embodiments, a liquid pharmaceutical formulation described herein has an osmolarity in the range of about 100 mOsm/kg to about 1600 mOsm/kg. In certain embodiments, a liquid pharmaceutical formulation described herein has an osmolarity in the range of about 200 mOsm/kg to about 800 mOsm/kg. In certain embodiments, a liquid pharmaceutical formulation described herein has an osmolarity in the range of about 200 mOsm/kg to about 600 mOsm/kg. In certain embodiments, a liquid pharmaceutical formulation described herein has an osmolarity in the range of about 200 mOsm/kg to about 400 mOsm/kg. In certain embodiments, a liquid pharmaceutical formulation described herein has an osmolarity in the range of from about 275 mOsm/kg to about 400 mOsm/kg.

It should be understood that the above ranges and description of osmolarity are equally applicable to osmolality but with the units of “Osm/kg.”

### (2) Water

In various embodiments, a liquid pharmaceutical formulation described herein further comprises water. In certain embodiments, the water is water for injection.

In various embodiments, a liquid pharmaceutical formulation described herein can comprise about 0.1 mL to about 2 mL, about 0.2 mL to about 2 mL, about 0.3 mL to about 2 mL, about 0.4 mL to about 2 mL, about 0.5 mL to about 2 mL, about 0.6 mL to about 2 mL, about 0.7 mL to about 2 mL, about 0.8 mL to about 2 mL, about 0.9 mL to about 2 mL, about 1 mL to about 2 mL, about 1.5 mL to about 2 mL, about 0.1 mL to about 1.5 mL, about 0.1 mL to about 1 mL, about 0.1 mL to about 0.9 mL, about 0.1 mL to about 0.8 mL, about 0.1 mL to about 0.7 mL, about 0.1 mL to about 0.6 mL, about 0.1 mL to about 0.5 mL, about 0.1 mL to about 0.4 mL, about 0.1 mL to about 0.3 mL, about 0.1 mL to about 0.2 mL, about 0.2 mL to about 1.5 mL, about 0.2 mL to about 1 mL, about 0.2 mL to about 0.9 mL, about 0.2 mL to about 0.8 mL, about 0.2 mL to about 0.7 mL, about 0.2 mL to about 0.6 mL, about 0.2 mL to about 0.5 mL, about 0.2 mL to about 0.4 mL, about 0.2 mL to about 0.3 mL, about 0.3 mL to about 1.5 mL, about 0.3 mL to about 1 mL, about 0.3 mL to about 0.9 mL, about 0.3 mL to about 0.8 mL, about 0.3 mL to about 0.7 mL, about 0.3 mL to about 0.6 mL, about 0.3 mL to about 0.5 mL, about 0.3 mL to about 0.4 mL, about 0.4 mL to about 1.5 mL, about 0.4 mL to about 1 mL, about 0.4 mL to about 0.9 mL, about 0.4 mL to about 0.8 mL, about 0.4 mL to about 0.7 mL, about 0.4 mL to about 0.6 mL, about 0.4 mL to about 0.5 mL, about 0.5 mL to about 1.5 mL, about 0.5 mL to about 1 mL, about 0.5 mL to about 0.9 mL, about 0.5 mL to about 0.8 mL, about 0.5 mL to about 0.7 mL, about 0.5 mL to about 0.6 mL, about 0.6 mL to about 1.5 mL, about 0.6 mL to about 1 mL, about 0.6 mL to about 0.9 mL, about 0.6 mL to about 0.8 mL, about 0.6 mL to about 0.7 mL, about 0.7 mL to about 1.5 mL, about 0.7 mL to about 1 mL, about 0.7 mL to about 0.9 mL, about 0.7 mL to about 0.8 mL, about 0.8 mL to about 1.5 mL, about 0.8 mL to about 1 mL, about 0.8 mL to about 0.9 mL, about 0.9 mL to about 1.5 mL, about 0.9 mL to about 1 mL, or about 1 mL to about 1.5 mL water.

In various embodiments, the liquid pharmaceutical compositions described herein can comprise about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, or about 2 mL water. In certain embodiments, the liquid pharmaceutical compositions described herein can comprise about 0.3 mL water. In certain embodiments, the liquid pharmaceutical compositions described herein can comprise about 0.4 mL water. In certain embodiments, the liquid pharmaceutical compositions described herein can comprise about 0.5 mL water. In certain embodiments, the liquid pharmaceutical compositions described herein can comprise about 0.6 mL water. In certain embodiments, the liquid pharmaceutical compositions described herein can comprise about 0.7 mL water. In certain embodiments, the liquid pharmaceutical compositions described herein can comprise about 0.8 mL water. In certain embodiments, the liquid pharmaceutical compositions described herein can comprise about 0.9 mL water. In certain embodiments, the liquid pharmaceutical compositions described herein can comprise about 1 mL water. In certain embodiments, the liquid pharmaceutical compositions described herein can comprise about 1.1 mL water. In certain embodiments, the liquid pharmaceutical compositions described herein can comprise about 1.2 mL water.

It should be understood that the volume of water added to the pharmaceutical formulation can be an amount to bring the total volume to one of the enumerated volumes of water herein, for example, to bring the total volume of the pharmaceutical formulation to about 0.1 mL, about 0.2 mL, about 0.3 mL, about 0.4 mL, 0.5 mL, 0.6 mL, 0.7 mL, 0.8 mL, 0.9 mL, or 1.0 mL.

In various embodiments, the liquid pharmaceutical compositions described herein can comprise about 0.1 g to about 2 g, about 0.2 g to about 2 g, about 0.3 g to about 2 g, about 0.4 g to about 2 g, about 0.5 g to about 2 g, about 0.6 g to about 2 g, about 0.7 g to about 2 g, about 0.8 g to about 2 g, about 0.9 g to about 2 g, about 1 g to about 2 g, about 1.5 g to about 2 g, about 0.1 g to about 1.5 g, about 0.1 g to about 1 g, about 0.1 g to about 0.9 g, about 0.1 g to about 0.8 g, about 0.1 g to about 0.7 g, about 0.1 g to about 0.6 g, about 0.1 g to about 0.5 g, about 0.1 g to about 0.4 g, about 0.1 g to about 0.3 g, about 0.1 g to about 0.2 g, about 0.2 g to about 1.5 g, about 0.2 g to about 1 g, about 0.2 g to about 0.9 g, about 0.2 g to about 0.8 g, about 0.2 g to about 0.7 g, about 0.2 g to about 0.6 g, about 0.2 g to about 0.5 g, about 0.2 g to about 0.4 g, about 0.2 g to about 0.3 g, about 0.3 g to about 1.5 g, about 0.3 g to about 1 g, about 0.3 g to about 0.9 g, about 0.3 g to about 0.8 g, about 0.3 g to about 0.7 g, about 0.3 g to about 0.6 g, about 0.3 g to about 0.5 g, about 0.3 g to about 0.4 g, about 0.4 g to about 1.5 g, about 0.4 g to about 1 g, about 0.4 g to about 0.9 g, about 0.4 g to about 0.8 g, about 0.4 g to about 0.7 g, about 0.4 g to about 0.6 g, about 0.4 g to about 0.5 g, about 0.5 g to about 1.5 g, about 0.5 g to about 1 g, about 0.5 g to about 0.9 g, about 0.5 g to about 0.8 g, about 0.5 g to about 0.7 g, about 0.5 g to about 0.6 g, about 0.6 g to about 1.5 g, about 0.6 g to about 1 g, about 0.6 g to about 0.9 g, about 0.6 g to about 0.8 g, about 0.6 g to about 0.7 g, about 0.7 g to about 1.5 g, about 0.7 g to about 1 g, about 0.7 g to about 0.9 g, about 0.7 g to about 0.8 g, about 0.8 g to about 1.5 g, about 0.8 g to about 1 g, about 0.8 g to about 0.9 g, about 0.9 g to about 1.5 g, about 0.9 g to about 1 g, or about 1 g to about 1.5 g water.

In various embodiments, the liquid pharmaceutical compositions described herein can comprise about 0.1 g, about 0.2 g, about 0.3 g, about 0.4 g, about 0.5 g, about 0.6 g, about 0.7 g, about 0.8 g, about 0.9 g, about 1 g, about 1.1 g, about 1.2 g, about 1.3 g, about 1.4 g, about 1.5 g, about 1.6 g, about 1.7 g, about 1.8 g, about 1.9 g, or about 2 g water. In certain embodiments, the liquid pharmaceutical compositions described herein can comprise about 0.3 g water. In certain embodiments, the liquid pharmaceutical compositions described herein can comprise about 0.4 g water. In certain embodiments, the liquid pharmaceutical compositions described herein can comprise about 0.5 g water. In certain embodiments, the liquid pharmaceutical compositions described herein can comprise about 0.6 g water. In certain embodiments, the liquid pharmaceutical compositions described herein can comprise about 0.7 g water. In certain embodiments, the liquid pharmaceutical compositions described herein can comprise about 0.8 g water. In certain embodiments, the liquid pharmaceutical compositions described herein can comprise about 0.9 g water. In certain embodiments, the liquid pharmaceutical compositions described herein can comprise about 1 g water. In certain embodiments, the liquid pharmaceutical compositions described herein can comprise about 1.1 g water. In certain embodiments, the liquid pharmaceutical compositions described herein can comprise about 1.2 g water.

### (3) Other Excipients

In various embodiments, the liquid pharmaceutical formulations described herein may further comprise one or more additional pharmaceutically acceptable excipients.

In various embodiments, the one or more additional pharmaceutically acceptable excipients is selected from the group consisting of ethanol, glycerin, N-methyl-pyrrolidone (NMP), sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In certain embodiments, the one or more additional pharmaceutically acceptable excipients comprises ethanol. In certain embodiments, the one or more additional pharmaceutically acceptable excipients comprises glycerin. In certain embodiments, the one or more additional pharmaceutically acceptable excipients comprises N-methyl-pyrrolidone (NMP). In certain embodiments, the one or more additional pharmaceutically acceptable excipients comprises sodium carbonate. In certain embodiments, the one or more additional pharmaceutically acceptable excipients comprises mannitol. In certain embodiments, the one or more additional pharmaceutically acceptable excipients comprises lactose. In certain embodiments, the one or more additional pharmaceutically acceptable excipients comprises dextrose. In certain embodiments, the one or more additional pharmaceutically acceptable excipients comprises a polyethylene glycol (PEG). In certain embodiments, the one or more additional pharmaceutically acceptable excipients comprises propylene glycol. In certain embodiments, the one or more additional pharmaceutically acceptable excipients comprises a polysorbate (e.g., polyoxyethylene (20) sorbitan monolaurate, polyoxyethylene (20) sorbitan monopalmitate, polyoxyethylene (20) sorbitan monostearate, polyoxyethylene (20) sorbitan monooleate). In certain embodiments, the one or more additional pharmaceutically acceptable excipients comprises a polyvinylpyrrolidone (PVP). In certain embodiments, the one or more additional pharmaceutically acceptable excipients comprises a cyclodextrin (e.g., an a (alpha)-cyclodextrin, a β (beta)-cyclodextrin, and a γ (gamma)-cyclodextrin). In certain embodiments, the one or more additional pharmaceutically acceptable excipients comprises a cyclodextrin derivative (e.g., a sulfobutyl-ether-β-cyclodextrin (e.g., captisol), a hydroxypropyl-β-cyclodextrin (e.g., 2-hydroxypropyl-β-cyclodextrin), and a methylated β-cyclodextrin (e.g., a randomly methylated β-cyclodextrin)). In certain embodiments, the one or more additional pharmaceutically acceptable excipients comprises vitamin E. In certain embodiments, the one or more additional pharmaceutically acceptable excipients comprises one or more naturally occurring forms of vitamin E (e.g., alpha-, beta-, gamma-, and delta-tocopherol and alpha-, beta-, gamma-, and delta-tocotrienol). In certain embodiments, the one or more additional pharmaceutically acceptable excipients comprises vitamin E derivative (e.g., tocopheryl acetate, tocopheryl glucoside, tocopheryl phosphate).

### (E) Formulation Volume

In various embodiments, the total volume of a liquid pharmaceutical formulation described herein can be about 0.1 mL to about 2 mL, about 0.2 mL to about 2 mL, about 0.3 mL to about 2 mL, about 0.4 mL to about 2 mL, about 0.5 mL to about 2 mL, about 0.6 mL to about 2 mL, about 0.7 mL to about 2 mL, about 0.8 mL to about 2 mL, about 0.9 mL to about 2 mL, about 1 mL to about 2 mL, about 1.5 mL to about 2 mL, about 0.1 mL to about 1.5 mL, about 0.1 mL to about 1 mL, about 0.1 mL to about 0.9 mL, about 0.1 mL to about 0.8 mL, about 0.1 mL to about 0.7 mL, about 0.1 mL to about 0.6 mL, about 0.1 mL to about 0.5 mL, about 0.1 mL to about 0.4 mL, about 0.1 mL to about 0.3 mL, about 0.1 mL to about 0.2 mL, about 0.2 mL to about 1.5 mL, about 0.2 mL to about 1 mL, about 0.2 mL to about 0.9 mL, about 0.2 mL to about 0.8 mL, about 0.2 mL to about 0.7 mL, about 0.2 mL to about 0.6 mL, about 0.2 mL to about 0.5 mL, about 0.2 mL to about 0.4 mL, about 0.2 mL to about 0.3 mL, about 0.3 mL to about 1.5 mL, about 0.3 mL to about 1 mL, about 0.3 mL to about 0.9 mL, about 0.3 mL to about 0.8 mL, about 0.3 mL to about 0.7 mL, about 0.3 mL to about 0.6 mL, about 0.3 mL to about 0.5 mL, about 0.3 mL to about 0.4 mL, about 0.4 mL to about 1.5 mL, about 0.4 mL to about 1 mL, about 0.4 mL to about 0.9 mL, about 0.4 mL to about 0.8 mL, about 0.4 mL to about 0.7 mL, about 0.4 mL to about 0.6 mL, about 0.4 mL to about 0.5 mL, about 0.5 mL to about 1.5 mL, about 0.5 mL to about 1 mL, about 0.5 mL to about 0.9 mL, about 0.5 mL to about 0.8 mL, about 0.5 mL to about 0.7 mL, about 0.5 mL to about 0.6 mL, about 0.6 mL to about 1.5 mL, about 0.6 mL to about 1 mL, about 0.6 mL to about 0.9 mL, about 0.6 mL to about 0.8 mL, about 0.6 mL to about 0.7 mL, about 0.7 mL to about 1.5 mL, about 0.7 mL to about 1 mL, about 0.7 mL to about 0.9 mL, about 0.7 mL to about 0.8 mL, about 0.8 mL to about 1.5 mL, about 0.8 mL to about 1 mL, about 0.8 mL to about 0.9 mL, about 0.9 mL to about 1.5 mL, about 0.9 mL to about 1 mL, or about 1 mL to about 1.5 mL.

In various embodiments, the total volume of a liquid pharmaceutical formulation described herein can be about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, or about 2 mL. In certain embodiments, the total volume of a liquid pharmaceutical formulation described herein is about 0.3 mL. In certain embodiments, the total volume of a liquid pharmaceutical formulation described herein is about 0.4 mL. In certain embodiments, the total volume of a liquid pharmaceutical formulation described herein is about 0.5 mL. In certain embodiments, the total volume of a liquid pharmaceutical formulation described herein is about 0.6 mL. In certain embodiments, the total volume of a liquid pharmaceutical formulation described herein is about 0.7 mL. In certain embodiments, the total volume of a liquid pharmaceutical formulation described herein is about 0.8 mL. In certain embodiments, the total volume of a liquid pharmaceutical formulation described herein is about 0.9 mL. In certain embodiments, the total volume of a liquid pharmaceutical formulation described herein is about 1 mL. In certain embodiments, the total volume of a liquid pharmaceutical formulation described herein is about 1.1 mL. In certain embodiments, the total volume of a liquid pharmaceutical formulation described herein is about 1.2 mL.

### (F) Exemplary Liquid Pharmaceutical Formulations

In various embodiments, a liquid pharmaceutical formulation can comprise:
(i) an effective amount of a loop diuretic, or a pharmaceutically acceptable salt thereof; and
(ii) a pharmaceutically acceptable buffer.

In various embodiments, a liquid pharmaceutical formulation can comprise:
(i) an effective amount of a loop diuretic, or a pharmaceutically acceptable salt thereof;
(ii) a pharmaceutically acceptable buffer; and
(iii) a solubilizing agent.

In various embodiments, a liquid pharmaceutical formulation can comprise:
(i) an effective amount of a loop diuretic, or a pharmaceutically acceptable salt thereof;
(ii) a pharmaceutically acceptable buffer;
(iii) a solubilizing agent; and
(iv) a pH adjuster.

In various embodiments, a liquid pharmaceutical formulation can comprise:
(i) an effective amount of a loop diuretic, or a pharmaceutically acceptable salt thereof;
(ii) a pharmaceutically acceptable buffer;
(iii) a solubilizing agent;
(iv) a pH adjuster; and
(v) water.

In various embodiments, a liquid pharmaceutical formulation can comprise:
(i) about 1 mg to about 100 mg of a loop diuretic, or a pharmaceutically acceptable salt thereof; and
(ii) about 4 mg to about 16 mg of a pharmaceutically acceptable buffer.

In various embodiments, a liquid pharmaceutical formulation can comprise:
(i) about 1 mg to about 100 mg of a loop diuretic, or a pharmaceutically acceptable salt thereof;
(ii) about 4 mg to about 16 mg of a pharmaceutically acceptable buffer; and
(iii) about 15 mg to about 50 mg of a solubilizing agent.

In various embodiments, a liquid pharmaceutical formulation can comprise:
(i) about 1 mg to about 100 mg of a loop diuretic, or a pharmaceutically acceptable salt thereof;
(ii) about 4 mg to about 16 mg of a pharmaceutically acceptable buffer;
(iii) about 15 mg to about 50 mg of a solubilizing agent; and
(iv) a pH adjuster.

In various embodiments, a liquid pharmaceutical formulation can comprise:
(i) about 1 mg to about 100 mg of a loop diuretic, or a pharmaceutically acceptable salt thereof;
(ii) about 4 mg to about 16 mg of a pharmaceutically acceptable buffer;
(iii) about 15 mg to about 50 mg of a solubilizing agent;
(iv) a pH adjuster; and
(v) water.

In various embodiments, a liquid pharmaceutical formulation comprises about 1 mg to about 100 mg of a loop diuretic, or a pharmaceutically acceptable salt thereof, has a pH of about 7 to about 8, and has a total volume of about 0.5 mL to about 1 mL.

### (1) Exemplary Furosemide Formulations

In various embodiments, a liquid pharmaceutical formulation can comprise:
(i) an effective amount of furosemide, or a pharmaceutically acceptable salt thereof; and
(ii) a pharmaceutically acceptable buffer.

In various embodiments, a liquid pharmaceutical formulation can comprise:
(i) an effective amount of furosemide, or a pharmaceutically acceptable salt thereof;
(ii) a pharmaceutically acceptable buffer; and
(iii) a solubilizing agent.

In various embodiments, a liquid pharmaceutical formulation can comprise:
(i) an effective amount of furosemide, or a pharmaceutically acceptable salt thereof;
(ii) a pharmaceutically acceptable buffer;
(iii) a solubilizing agent; and
(iv) a pH adjuster.

In various embodiments, a liquid pharmaceutical formulation can comprise:
(i) an effective amount of furosemide, or a pharmaceutically acceptable salt thereof;
(ii) a pharmaceutically acceptable buffer;
(iii) a solubilizing agent;
(iv) a pH adjuster; and
(v) water.

In various embodiments, a liquid pharmaceutical formulation can comprise:
(i) an effective amount of furosemide, or a pharmaceutically acceptable salt thereof; and
(ii) tromethamine.

In various embodiments, a liquid pharmaceutical formulation can comprise:
(i) an effective amount of furosemide, or a pharmaceutically acceptable salt thereof;
(ii) tromethamine; and
(iii) benzyl alcohol.

In various embodiments, a liquid pharmaceutical formulation can comprise:
(i) an effective amount of furosemide, or a pharmaceutically acceptable salt thereof;
(ii) tromethamine;
(iii) benzyl alcohol;
(iv) hydrochloric acid; and
(v) sodium hydroxide.

In various embodiments, a liquid pharmaceutical formulation can comprise:
(i) an effective amount of furosemide, or a pharmaceutically acceptable salt thereof;
(ii) tromethamine;
(iii) benzyl alcohol;
(iv) hydrochloric acid;
(v) sodium hydroxide; and
(v) water.

In various embodiments, a liquid pharmaceutical formulation can comprise:
(i) about 5% (w/w) to about 10% (w/w) furosemide, or a pharmaceutically acceptable salt thereof; and
(ii) about 0.1% (w/w) to about 1.5% (w/w) of a pharmaceutically acceptable buffer.

In various embodiments, a liquid pharmaceutical formulation can comprise:
(i) about 5% (w/w) to about 10% (w/w) furosemide, or a pharmaceutically acceptable salt thereof;
(ii) about 0.1% (w/w) to about 1.5% (w/w) of a pharmaceutically acceptable buffer; and
(iii) about 1% (w/w) to about 5% (w/w) of a solubilizing agent.

In various embodiments, a liquid pharmaceutical formulation can comprise:
(i) about 5% (w/w) to about 10% (w/w) furosemide, or a pharmaceutically acceptable salt thereof;
(ii) about 0.1% (w/w) to about 1.5% (w/w) of a pharmaceutically acceptable buffer;
(iii) about 1% (w/w) to about 5% (w/w) of a solubilizing agent; and
(iv) a pH adjuster.

In various embodiments, a liquid pharmaceutical formulation can comprise:
(i) about 5% (w/w) to about 10% (w/w) furosemide, or a pharmaceutically acceptable salt thereof;
(ii) about 0.1% (w/w) to about 1.5% (w/w) of a pharmaceutically acceptable buffer;
(iii) about 1% (w/w) to about 5% (w/w) of a solubilizing agent;
(iv) a pH adjuster; and
(v) water.

In various embodiments, a liquid pharmaceutical formulation can comprise:
(i) about 40 mg to about 100 mg furosemide, or a pharmaceutically acceptable salt thereof; and
(ii) about 4 mg to about 16 mg of a pharmaceutically acceptable buffer.

In various embodiments, a liquid pharmaceutical formulation can comprise:
(i) about 40 mg to about 100 mg furosemide, or a pharmaceutically acceptable salt thereof;
(ii) about 4 mg to about 16 mg of a pharmaceutically acceptable buffer; and
(iii) about 15 mg to about 50 mg of a solubilizing agent.

In various embodiments, a liquid pharmaceutical formulation can comprise:
(i) about 40 mg to about 100 mg furosemide, or a pharmaceutically acceptable salt thereof;
(ii) about 4 mg to about 16 mg of a pharmaceutically acceptable buffer;
(iii) about 15 mg to about 50 mg of a solubilizing agent; and
(iv) a pH adjuster.

In various embodiments, a liquid pharmaceutical formulation can comprise:
(i) about 40 mg to about 100 mg furosemide, or a pharmaceutically acceptable salt thereof;
(ii) about 4 mg to about 16 mg of a pharmaceutically acceptable buffer;
(iii) about 15 mg to about 50 mg of a solubilizing agent;
(iv) a pH adjuster; and
(v) water.

In various embodiments, a liquid pharmaceutical formulation can comprise:
(i) about 5% (w/w) to about 10% (w/w) furosemide, or a pharmaceutically acceptable salt thereof; and
(ii) about 0.1% (w/w) to about 1.5% (w/w) tromethamine.

In various embodiments, a liquid pharmaceutical formulation can comprise:
(i) about 5% (w/w) to about 10% (w/w) furosemide, or a pharmaceutically acceptable salt thereof;
(ii) about 0.1% (w/w) to about 1.5% (w/w) tromethamine; and
(iii) about 1% (w/w) to about 5% (w/w) benzyl alcohol.

In various embodiments, a liquid pharmaceutical formulation can comprise:
(i) about 5% (w/w) to about 10% (w/w) furosemide, or a pharmaceutically acceptable salt thereof;
(ii) about 0.1% (w/w) to about 1.5% (w/w) tromethamine;
(iii) about 1% (w/w) to about 5% (w/w) benzyl alcohol; and
(iv) a pH adjuster.

In various embodiments, a liquid pharmaceutical formulation can comprise:
(i) about 5% (w/w) to about 10% (w/w) furosemide, or a pharmaceutically acceptable salt thereof;
(ii) about 0.1% (w/w) to about 1.5% (w/w) tromethamine;
(iii) about 1% (w/w) to about 5% (w/w) benzyl alcohol;
(iv) a pH adjuster; and
(v) water.

In various embodiments, a liquid pharmaceutical formulation can comprise:
(i) about 40 mg to about 100 mg furosemide, or a pharmaceutically acceptable salt thereof; and
(ii) about 4 mg to about 16 mg tromethamine.

In various embodiments, a liquid pharmaceutical formulation can comprise:
(i) about 40 mg to about 100 mg furosemide, or a pharmaceutically acceptable salt thereof;
(ii) about 4 mg to about 16 mg tromethamine; and
(iii) about 15 mg to about 50 mg benzyl alcohol.

In various embodiments, a liquid pharmaceutical formulation can comprise:
(i) about 40 mg to about 100 mg furosemide, or a pharmaceutically acceptable salt thereof;
(ii) about 4 mg to about 16 mg tromethamine;
(iii) about 15 mg to about 50 mg benzyl alcohol;
(iv) hydrochloric acid; and
(v) sodium hydroxide.

In various embodiments, a liquid pharmaceutical formulation can comprise:
(i) about 40 mg to about 100 mg furosemide, or a pharmaceutically acceptable salt thereof;
(ii) about 4 mg to about 16 mg tromethamine;
(iii) about 15 mg to about 50 mg benzyl alcohol;
(iv) hydrochloric acid;
(v) sodium hydroxide; and
(v) water.

In various embodiments, a liquid pharmaceutical formulation comprises about 40 mg to about 100 mg furosemide, has a pH of about 7 to about 8, and has a total volume of about 0.5 mL to about 1 mL.

### (2) Exemplary Torsemide Formulations

In various embodiments, a liquid pharmaceutical formulation can comprise:
(i) an effective amount of torsemide, or a pharmaceutically acceptable salt thereof; and
(ii) a pharmaceutically acceptable buffer.

In various embodiments, a liquid pharmaceutical formulation can comprise:
(i) an effective amount of torsemide, or a pharmaceutically acceptable salt thereof;
(ii) a pharmaceutically acceptable buffer; and
(iii) a solubilizing agent.

In various embodiments, a liquid pharmaceutical formulation can comprise:
(i) an effective amount of torsemide, or a pharmaceutically acceptable salt thereof;
(ii) a pharmaceutically acceptable buffer;
(iii) a solubilizing agent; and
(iv) a pH adjuster.

In various embodiments, a liquid pharmaceutical formulation can comprise:
(i) an effective amount of torsemide, or a pharmaceutically acceptable salt thereof;
(ii) a pharmaceutically acceptable buffer;
(iii) a solubilizing agent;
(iv) a pH adjuster; and
(v) water.

In various embodiments, a liquid pharmaceutical formulation can comprise:
(i) about 20 mg to about 40 mg torsemide, or a pharmaceutically acceptable salt thereof; and
(ii) a pharmaceutically acceptable buffer.

In various embodiments, a liquid pharmaceutical formulation can comprise:
(i) about 20 mg to about 40 mg of torsemide, or a pharmaceutically acceptable salt thereof;
(ii) a pharmaceutically acceptable buffer; and
(iii) a solubilizing agent.

In various embodiments, a liquid pharmaceutical formulation can comprise:
(i) about 20 mg to about 40 mg torsemide, or a pharmaceutically acceptable salt thereof;
(ii) a pharmaceutically acceptable buffer;
(iii) a solubilizing agent; and
(iv) a pH adjuster.

In various embodiments, a liquid pharmaceutical formulation can comprise:
(i) about 20 mg to about 40 mg torsemide, or a pharmaceutically acceptable salt thereof;
(ii) a pharmaceutically acceptable buffer;
(iii) a solubilizing agent;
(iv) a pH adjuster; and
(v) water.

In various embodiments, a liquid pharmaceutical formulation comprises about 20 mg to about 40 mg torsemide, has a pH of about 7 to about 8, and has a total volume of about 0.5 mL to about 1 mL.

### (3) Exemplary Bumetanide Formulations

In various embodiments, a liquid pharmaceutical formulation can comprise:
(i) an effective amount of bumetanide, or a pharmaceutically acceptable salt thereof; and
(ii) a pharmaceutically acceptable buffer.

In various embodiments, a liquid pharmaceutical formulation can comprise:
(i) an effective amount of bumetanide, or a pharmaceutically acceptable salt thereof;
(ii) a pharmaceutically acceptable buffer; and
(iii) a solubilizing agent.

In various embodiments, a liquid pharmaceutical formulation can comprise:
(i) an effective amount of bumetanide, or a pharmaceutically acceptable salt thereof;
(ii) a pharmaceutically acceptable buffer;
(iii) a solubilizing agent; and
(iv) a pH adjuster.

In various embodiments, a liquid pharmaceutical formulation can comprise:
(i) an effective amount of bumetanide, or a pharmaceutically acceptable salt thereof;
(ii) a pharmaceutically acceptable buffer;
(iii) a solubilizing agent;
(iv) a pH adjuster; and
(v) water.

In various embodiments, a liquid pharmaceutical formulation can comprise:
(i) about 1 mg to about 2 mg bumetanide, or a pharmaceutically acceptable salt thereof; and
(ii) a pharmaceutically acceptable buffer.

In various embodiments, a liquid pharmaceutical formulation can comprise:
(i) about 1 mg to about 2 mg of bumetanide, or a pharmaceutically acceptable salt thereof;
(ii) a pharmaceutically acceptable buffer; and
(iii) a solubilizing agent.

In various embodiments, a liquid pharmaceutical formulation can comprise:
(i) about 1 mg to about 2 mg bumetanide, or a pharmaceutically acceptable salt thereof;
(ii) a pharmaceutically acceptable buffer;
(iii) a solubilizing agent; and
(iv) a pH adjuster.

In various embodiments, a liquid pharmaceutical formulation can comprise:
(i) about 1 mg to about 2 mg bumetanide, or a pharmaceutically acceptable salt thereof;
(ii) a pharmaceutically acceptable buffer;
(iii) a solubilizing agent;
(iv) a pH adjuster; and
(v) water.

In various embodiments, a liquid pharmaceutical formulation comprises about 1 mg to about 2 mg bumetanide, has a pH of about 7 to about 8, and has a total volume of about 0.5 mL to about 1 mL.

### Methods of Treatment

In one aspect, a liquid pharmaceutical formulation described herein may be used for the treatment or prevention of a variety of conditions such as, but not limited to, congestion due to fluid overload, edema, and hypertension in a patient in need thereof. In various embodiments, the condition is selected from the group consisting of congestion due to fluid overload, edema, and hypertension, and combinations thereof. In certain embodiments, the condition is congestion due to fluid overload. In certain embodiments, the condition is edema. In certain embodiments, the condition is hypertension. In certain embodiments, the edema is associated with congestive heart failure, cirrhosis of the liver or renal disease. In certain embodiments, the renal disease is nephrotic syndrome.

In certain embodiments, the patient is a human.

In various embodiments, provided herein is a method of treating a condition selected from the group consisting of congestion due to fluid overload, edema, and hypertension, in a human in need thereof, administering to the human via subcutaneous injection about 1 mL or less of a liquid pharmaceutical formulation described herein.

In various embodiments, provided herein is a method of treating a condition selected from the group consisting of congestion due to fluid overload, edema, and hypertension, in a human in need thereof, administering to the human via subcutaneous injection an effective amount of a loop diuretic in a total volume of about 1 mL or less of a liquid pharmaceutical formulation.

### (A) Effective Amounts/Concentrations of Loop Diuretics

In various embodiments, the effective amount of the loop diuretic in the total volume of the liquid pharmaceutical formulation is about 1 mg to about 100 mg, about 2 mg to about 100 mg, about 5 mg to about 100 mg, about 10 mg to about 100 mg, about 20 mg to about 100 mg, about 30 mg to about 100 mg, about 40 mg to about 100 mg, about 50 mg to about 100 mg, about 60 mg to about 100 mg, about 70 mg to about 100 mg, about 80 mg to about 100 mg, about 1 mg to about 80 mg, about 1 mg to about 70 mg, about 1 mg to about 60 mg, about 1 mg to about 50 mg, about 1 mg to about 40 mg, about 1 mg to about 30 mg, about 1 mg to about 20 mg, about 1 mg to about 10 mg, about 1 mg to about 5 mg, about 1 mg to about 2 mg, about 2 mg to about 80 mg, about 2 mg to about 70 mg, about 2 mg to about 60 mg, about 2 mg to about 50 mg, about 2 mg to about 40 mg, about 2 mg to about 30 mg, about 2 mg to about 20 mg, about 2 mg to about 10 mg, about 2 mg to about 5 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 10 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 70 mg, about 20 mg to about 60 mg, about 20 mg to about 50 mg, about 20 mg to about 40 mg, about 20 mg to about 30 mg, about 30 mg to about 80 mg, about 30 mg to about 70 mg, about 30 mg to about 60 mg, about 30 mg to about 50 mg, about 30 mg to about 40 mg, about 40 mg to about 80 mg, about 40 mg to about 70 mg, about 40 mg to about 60 mg, about 40 mg to about 50 mg, about 50 mg to about 80 mg, about 50 mg to about 70 mg, about 50 mg to about 60 mg, about 60 mg to about 80 mg, about 60 mg to about 70 mg, or about 70 mg to about 80 mg. In certain embodiments, the effective amount of the loop diuretic in the total volume of the liquid pharmaceutical formulation is about 1 mg to about 100 mg. In certain embodiments, the effective amount of the loop diuretic in the total volume of the liquid pharmaceutical formulation is about 1 mg to about 80 mg. In certain embodiments, the effective amount of the loop diuretic in the total volume of the liquid pharmaceutical formulation is about 2 mg to about 80 mg. In certain embodiments, the effective amount of the loop diuretic in the total volume of the liquid pharmaceutical formulation is about 40 mg to about 100 mg. In certain embodiments, the effective amount of the loop diuretic in the total volume of the liquid pharmaceutical formulation is about 1 mg to about 40 mg. In certain embodiments, the effective amount of the loop diuretic in the total volume of the liquid pharmaceutical formulation is about 40 mg to about 80 mg.

In certain embodiments, the effective amount of the loop diuretic in the total volume of the liquid pharmaceutical formulation is about 1 mg, about 2 mg, about 3 mg, about 4 mg, about 5 mg, about 6 mg, about 7 mg, about 8 mg, about 9 mg, about 10 mg, about 12 mg, about 14 mg, about 16 mg, about 18 mg, about 20 mg, about 22 mg, about 24 mg, about 26 mg, about 28 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In certain embodiments, the effective amount of the loop diuretic in the total volume of the liquid pharmaceutical formulation is about 1 mg. In certain embodiments, the effective amount of the loop diuretic in the total volume of the liquid pharmaceutical formulation is about 2 mg. In certain embodiments, the effective amount of the loop diuretic in the total volume of the liquid pharmaceutical formulation is about 20 mg. In certain embodiments, the effective amount of the loop diuretic in the total volume of the liquid pharmaceutical formulation is about 40 mg. In certain embodiments, the effective amount of the loop diuretic in the total volume of the liquid pharmaceutical formulation is about 80 mg. In certain embodiments, the effective amount of the loop diuretic in the total volume of the liquid pharmaceutical formulation is about 100 mg.

In various embodiments, the concentration of the loop diuretic in the total volume of the liquid pharmaceutical formulation is about 2 mg/mL to about 100 mg/mL, about 5 mg/mL to about 100 mg/mL, about 10 mg/mL to about 100 mg/mL, about 20 mg/mL to about 100 mg/mL, about 30 mg/mL to about 100 mg/mL, about 40 mg/mL to about 100 mg/mL, about 50 mg/mL to about 100 mg/mL, about 60 mg/mL to about 100 mg/mL, about 70 mg/mL to about 100 mg/mL, about 80 mg/mL to about 100 mg/mL, about 2 mg/mL to about 80 mg/mL, about 2 mg/mL to about 70 mg/mL, about 2 mg/mL to about 60 mg/mL, about 2 mg/mL to about 50 mg/mL, about 2 mg/mL to about 40 mg/mL, about 2 mg/mL to about 30 mg/mL, about 2 mg/mL to about 20 mg/mL, about 2 mg/mL to about 10 mg/mL, about 2 mg/mL to about 5 mg/mL, about 5 mg/mL to about 80 mg/mL, about 5 mg/mL to about 70 mg/mL, about 5 mg/mL to about 60 mg/mL, about 5 mg/mL to about 50 mg/mL, about 5 mg/mL to about 40 mg/mL, about 5 mg/mL to about 30 mg/mL, about 5 mg/mL to about 20 mg/mL, about 5 mg/mL to about 10 mg/mL, about 10 mg/mL to about 80 mg/mL, about 10 mg/mL to about 70 mg/mL, about 10 mg/mL to about 60 mg/mL, about 10 mg/mL to about 50 mg/mL, about 10 mg/mL to about 40 mg/mL, about 10 mg/mL to about 30 mg/mL, about 10 mg/mL to about 20 mg/mL, about 20 mg/mL to about 80 mg/mL, about 20 mg/mL to about 70 mg/mL, about 20 mg/mL to about 60 mg/mL, about 20 mg/mL to about 50 mg/mL, about 20 mg/mL to about 40 mg/mL, about 20 mg/mL to about 30 mg/mL, about 30 mg/mL to about 80 mg/mL, about 30 mg/mL to about 70 mg/mL, about 30 mg/mL to about 60 mg/mL, about 30 mg/mL to about 50 mg/mL, about 30 mg/mL to about 40 mg/mL, about 40 mg/mL to about 80 mg/mL, about 40 mg/mL to about 70 mg/mL, about 40 mg/mL to about 60 mg/mL, about 40 mg/mL to about 50 mg/mL, about 50 mg/mL to about 80 mg/mL, about 50 mg/mL to about 70 mg/mL, about 50 mg/mL to about 60 mg/mL, about 60 mg/mL to about 80 mg/mL, about 60 mg/mL to about 70 mg/mL, or about 70 mg/mL to about 80 mg/mL. In certain embodiments, the concentration of the loop diuretic in the total volume of the liquid pharmaceutical formulation is about 2 mg/mL to about 100 mg/mL.

In various embodiments, the concentration of the loop diuretic in the total volume of the liquid pharmaceutical formulation is about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 22 mg/mL, about 24 mg/mL, about 26 mg/mL, about 28 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL. In certain embodiments, the concentration of the loop diuretic in the total volume of the liquid pharmaceutical formulation is about 2 mg/mL. In certain embodiments, the concentration of the loop diuretic in the total volume of the liquid pharmaceutical formulation is about 40 mg/mL. In certain embodiments, the concentration of the loop diuretic in the total volume of the liquid pharmaceutical formulation is about 80 mg/mL.

In various embodiments, the loop diuretic is bumetanide, furosemide, or torsemide. In certain embodiments, the loop diuretic is bumetanide. In certain embodiments, the loop diuretic is furosemide. In certain embodiments, the loop diuretic is torsemide.

In various embodiments, the total volume of the liquid pharmaceutical formulation is about 0.3 mL or less, about 0.4 mL or less, about 0.5 mL or less, about 0.6 mL or less, about 0.7 mL or less, about 0.8 mL or less, about 0.9 mL or less, or about 1 mL or less.

In various embodiments, the total volume of the liquid pharmaceutical formulation is about 0.3 mL, about 0.4 mL, about 0.5 mL, about 0.6 mL, about 0.7 mL, about 0.8 mL, about 0.9 mL, or about 1 mL. In certain embodiments, the total volume of the liquid pharmaceutical formulation is about 0.5 mL. In certain embodiments, the total volume of the liquid pharmaceutical formulation is about 1 mL.

### (1) Furosemide

In various embodiments, provided herein is a method of treating a condition selected from the group consisting of congestion due to fluid overload, edema, and hypertension, in a human in need thereof, comprising administering to the human via subcutaneous injection an effective amount of furosemide in a total volume of about 1 mL or less of a liquid pharmaceutical formulation.

In various embodiments, the effective amount of furosemide in the total volume of the liquid pharmaceutical formulation is about 40 mg to about 80 mg, about 50 mg to about 80 mg, about 60 mg to about 80 mg, about 70 mg to about 80 mg, about 40 mg to about 70 mg, about 40 mg to about 60 mg, about 40 mg to about 50 mg, about 50 mg to about 70 mg, about 50 mg to about 60 mg, or about 50 mg to about 60 mg. In certain embodiments, the effective amount of furosemide in the total volume of the liquid pharmaceutical formulation is about 40 mg to about 80 mg.

In various embodiments, the effective amount of furosemide in the total volume of the liquid pharmaceutical formulation is about 40 mg, about 45 mg, about 50 mg, about 55 mg, about 60 mg, about 65 mg, about 70 mg, about 75 mg, or about 80 mg. In certain embodiments, the effective amount of furosemide in the total volume of the liquid pharmaceutical formulation is about 40 mg. In certain embodiments, the effective amount of furosemide in the total volume of the liquid pharmaceutical formulation is about 80 mg.

In various embodiments, the concentration of furosemide in the total volume of the liquid pharmaceutical formulation is about 40 mg/mL, about 50 mg/mL, about 60 mg/mL, about 70 mg/mL, about 80 mg/mL, about 90 mg/mL, or about 100 mg/mL. In certain embodiments, the concentration of furosemide in the total volume of the liquid pharmaceutical formulation is about 80 mg/mL.

In various embodiments, provided herein is a method of treating a condition selected from the group consisting of congestion due to fluid overload, edema, and hypertension, in a human in need thereof, comprising administering to the human via subcutaneous injection about 80 mg of furosemide in a total volume of about 1 mL of a liquid pharmaceutical formulation.

In various embodiments, provided herein is a method of treating a condition selected from the group consisting of congestion due to fluid overload, edema, and hypertension, in a human in need thereof, comprising administering to the human via subcutaneous injection about 40 mg of furosemide in a total volume of about 0.5 mL of a liquid pharmaceutical formulation.

### (2) Bumetanide

In various embodiments, provided herein is a method of treating a condition selected from the group consisting of congestion due to fluid overload, edema, and hypertension, in a human in need thereof, comprising administering to the human via subcutaneous injection an effective amount of bumetanide in a total volume of about 1 mL or less of a liquid pharmaceutical formulation.

In various embodiments, the effective amount of bumetanide in the total volume of the liquid pharmaceutical formulation is about 1 mg to about 2 mg, about 1.2 mg to about 2 mg, about 1.4 mg to about 2 mg, about 1.6 mg to about 2 mg, about 1.8 mg to about 2 mg, about 1 mg to about 1.8 mg, about 1 mg to about 1.6 mg, about 1 mg to about 1.4 mg, about 1 mg to about 1.2 mg, about 1.2 mg to about 1.8 mg, about 1.2 mg to about 1.6 mg, about 1.2 mg to about 1.4 mg, about 1.4 mg to about 1.8 mg, about 1.4 mg to about 1.6 mg, or about 1.6 mg to about 1.8 mg. In certain embodiments, the effective amount of bumetanide in the total volume of the liquid pharmaceutical formulation is about 1 mg to about 2 mg.

In various embodiments, the effective amount of bumetanide in the total volume of the liquid pharmaceutical formulation is about 1 mg, about 1.1 mg, about 1.2 mg, about 1.3 mg, about 1.4 mg, about 1.5 mg, about 1.6 mg, about 1.7 mg, about 1.8 mg, about 1.9 mg, or about 2 mg. In certain embodiments, the effective amount of bumetanide in the total volume of the liquid pharmaceutical formulation is about 1 mg. In certain embodiments, the effective amount of bumetanide in the total volume of the liquid pharmaceutical formulation is about 2 mg.

In various embodiments, the concentration of bumetanide in the total volume of the liquid pharmaceutical formulation is about 1 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, or about 2 mg/mL. In certain embodiments, the concentration of bumetanide in the total volume of the liquid pharmaceutical formulation is about 2 mg/mL.

In various embodiments, provided herein is a method of treating a condition selected from the group consisting of congestion due to fluid overload, edema, and hypertension, in a human in need thereof, comprising administering to the human via subcutaneous injection about 2 mg of bumetanide in a total volume of about 1 mL of a liquid pharmaceutical formulation.

In various embodiments, provided herein is a method of treating a condition selected from the group consisting of congestion due to fluid overload, edema, and hypertension, in a human in need thereof, comprising administering to the human via subcutaneous injection about 1 mg of bumetanide in a total volume of about 0.5 mL of a liquid pharmaceutical formulation.

### (3) Torsemide

In various embodiments, provided herein is a method of treating a condition selected from the group consisting of congestion due to fluid overload, edema, and hypertension, in a human in need thereof, comprising administering to the human via subcutaneous injection an effective amount of torsemide in a total volume of about 1 mL or less of a liquid pharmaceutical formulation.

In various embodiments, the effective amount of torsemide in the total volume of the liquid pharmaceutical formulation is about 20 mg to about 40 mg, about 24 mg to about 40 mg, about 28 mg to about 40 mg, about 32 mg to about 40 mg, about 36 mg to about 40 mg, about 20 mg to about 36 mg, about 20 mg to about 32 mg, about 20 mg to about 28 mg, about 20 mg to about 24 mg, about 24 mg to about 36 mg, about 24 mg to about 32 mg, about 24 mg to about 28 mg, about 28 mg to about 36 mg, about 28 mg to about 32 mg, or about 32 mg to about 36 mg. In certain embodiments, the effective amount of torsemide in the total volume of the liquid pharmaceutical formulation is about 20 mg to about 40 mg.

In various embodiments, the effective amount of torsemide in the total volume of the liquid pharmaceutical formulation is about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, or about 40 mg. In certain embodiments, the effective amount of torsemide in the total volume of the liquid pharmaceutical formulation is about 20 mg. In certain embodiments, the effective amount of torsemide in the total volume of the liquid pharmaceutical formulation is about 40 mg.

In various embodiments, the concentration of torsemide in the total volume of the liquid pharmaceutical formulation is about 20 mg/mL, about 22 mg/mL, about 24 mg/mL, about 26 mg/mL, about 28 mg/mL, about 30 mg/mL, about 32 mg/mL, about 34 mg/mL, about 36 mg/mL, about 38 mg/mL, or about 40 mg/mL. In certain embodiments, the concentration of torsemide in the total volume of the liquid pharmaceutical formulation is about 40 mg/mL.

In various embodiments, provided herein is a method of treating a condition selected from the group consisting of congestion due to fluid overload, edema, and hypertension, in a human in need thereof, comprising administering to the human via subcutaneous injection about 40 mg of torsemide in a total volume of about 1 mL of a liquid pharmaceutical formulation.

In various embodiments, provided herein is a method of treating a condition selected from the group consisting of congestion due to fluid overload, edema, and hypertension, in a human in need thereof, comprising administering to the human via subcutaneous injection about 20 mg of torsemide in a total volume of about 0.5 mL of a liquid pharmaceutical formulation.

### (B) Pharmacokinetics/Pharmacodynamics

### (1) Urine Output

In various embodiments, a method described herein results in a total urine output of the human about 6 hours after administration is complete of about 500 mL to about 5000 mL, about 600 mL to about 5000 mL, about 700 mL to about 5000 mL, about 800 mL to about 5000 mL, about 900 mL to about 5000 mL, about 1000 mL to about 5000 mL, about 1500 mL to about 5000 mL, about 2000 mL to about 5000 mL, about 2500 mL to about 5000 mL, about 3000 mL to about 5000 mL, about 3500 mL to about 5000 mL, about 4000 mL to about 5000 mL, about 4500 mL to about 5000 mL, about 500 mL to about 4500 mL, about 500 mL to about 4000 mL, about 500 mL to about 3500 mL, about 500 mL to about 3000 mL, about 500 mL to about 2500 mL, about 500 mL to about 2000 mL, about 500 mL to about 1500 mL, about 500 mL to about 1000 mL, about 500 mL to about 900 mL, about 500 mL to about 800 mL, about 500 mL to about 700 mL, about 500 mL to about 600 mL, about 600 mL to about 4500 mL, about 600 mL to about 4000 mL, about 600 mL to about 3500 mL, about 600 mL to about 3000 mL, about 600 mL to about 2500 mL, about 600 mL to about 2000 mL, about 600 mL to about 1500 mL, about 600 mL to about 1000 mL, about 600 mL to about 900 mL, about 600 mL to about 800 mL, about 600 mL to about 700 mL, about 700 mL to about 4500 mL, about 700 mL to about 4000 mL, about 700 mL to about 3500 mL, about 700 mL to about 3000 mL, about 700 mL to about 2500 mL, about 700 mL to about 2000 mL, about 700 mL to about 1500 mL, about 700 mL to about 1000 mL, about 700 mL to about 800 mL, about 800 mL to about 4500 mL, about 800 mL to about 4000 mL, about 800 mL to about 3500 mL, about 800 mL to about 3000 mL, about 800 mL to about 2500 mL, about 800 mL to about 2000 mL, about 800 mL to about 1500 mL, about 800 mL to about 1000 mL, about 800 mL to about 900 mL, about 900 mL to about 4500 mL, about 900 mL to about 4000 mL, about 900 mL to about 3500 mL, about 900 mL to about 3000 mL, about 900 mL to about 2500 mL, about 900 mL to about 2000 mL, about 900 mL to about 1500 mL, about 900 mL to about 1000 mL, about 1000 mL to about 4500 mL, about 1000 mL to about 4000 mL, about 1000 mL to about 3500 mL, about 1000 mL to about 3000 mL, about 1000 mL to about 2500 mL, about 1000 mL to about 2000 mL, about 1000 mL to about 1500 mL, about 1500 mL to about 4500 mL, about 1500 mL to about 4000 mL, about 1500 mL to about 3500 mL, about 1500 mL to about 3000 mL, about 1500 mL to about 2500 mL, about 1500 mL to about 2000 mL, about 2000 mL to about 4500 mL, about 2000 mL to about 4000 mL, about 2000 mL to about 3500 mL, about 2000 mL to about 3000 mL, about 2000 mL to about 2500 mL, about 2500 mL to about 4500 mL, about 2500 mL to about 4000 mL, about 2500 mL to about 3500 mL, about 2500 mL to about 3000 mL, about 3000 mL to about 4500 mL, about 3000 mL to about 4000 mL, about 3000 mL to about 3500 mL, about 3500 mL to about 4500 mL, about 3500 mL to about 4000 mL, or about 4000 mL to about 4500 mL. In certain embodiments, a method described herein results in a total urine output of the human about 6 hours after administration is complete of about 600 mL to about 5000 mL. In certain embodiments, a method described herein results in a total urine output of the human about 6 hours after administration is complete of about 1200 mL to about 5000 mL. In certain embodiments, a method described herein results in a total urine output of the human about 6 hours after administration is complete of about 600 mL to about 2500 mL.

In various embodiments, a method described herein results in a total urine output of the human about 8 hours after administration is complete of about 500 mL to about 5500 mL, about 600 mL to about 5500 mL, about 700 mL to about 5500 mL, about 800 mL to about 5500 mL, about 900 mL to about 5500 mL, about 1000 mL to about 5500 mL, about 1500 mL to about 5500 mL, about 2000 mL to about 5500 mL, about 2500 mL to about 5500 mL, about 3000 mL to about 5500 mL, about 3500 mL to about 5500 mL, about 4000 mL to about 5500 mL, about 4500 mL to about 5500 mL, about 5000 mL to about 5500 mL, about 500 mL to about 5000 mL, about 500 mL to about 4500 mL, about 500 mL to about 4000 mL, about 500 mL to about 3500 mL, about 500 mL to about 3000 mL, about 500 mL to about 2500 mL, about 500 mL to about 2000 mL, about 500 mL to about 1500 mL, about 500 mL to about 1000 mL, about 500 mL to about 900 mL, about 500 mL to about 800 mL, about 500 mL to about 700 mL, about 500 mL to about 600 mL, about 600 mL to about 5000 mL, about 600 mL to about 4500 mL, about 600 mL to about 4000 mL, about 600 mL to about 3500 mL, about 600 mL to about 3000 mL, about 600 mL to about 2500 mL, about 600 mL to about 2000 mL, about 600 mL to about 1500 mL, about 600 mL to about 1000 mL, about 600 mL to about 900 mL, about 600 mL to about 800 mL, about 600 mL to about 700 mL, about 700 mL to about 5000 mL, about 700 mL to about 4500 mL, about 700 mL to about 4000 mL, about 700 mL to about 3500 mL, about 700 mL to about 3000 mL, about 700 mL to about 2500 mL, about 700 mL to about 2000 mL, about 700 mL to about 1500 mL, about 700 mL to about 1000 mL, about 700 mL to about 800 mL, about 800 mL to about 5000 mL, about 800 mL to about 4500 mL, about 800 mL to about 4000 mL, about 800 mL to about 3500 mL, about 800 mL to about 3000 mL, about 800 mL to about 2500 mL, about 800 mL to about 2000 mL, about 800 mL to about 1500 mL, about 800 mL to about 1000 mL, about 800 mL to about 900 mL, about 900 mL to about 5000 mL, about 900 mL to about 4500 mL, about 900 mL to about 4000 mL, about 900 mL to about 3500 mL, about 900 mL to about 3000 mL, about 900 mL to about 2500 mL, about 900 mL to about 2000 mL, about 900 mL to about 1500 mL, about 900 mL to about 1000 mL, about 1000 mL to about 5000 mL, about 1000 mL to about 4500 mL, about 1000 mL to about 4000 mL, about 1000 mL to about 3500 mL, about 1000 mL to about 3000 mL, about 1000 mL to about 2500 mL, about 1000 mL to about 2000 mL, about 1000 mL to about 1500 mL, about 1500 mL to about 5000 mL, about 1500 mL to about 4500 mL, about 1500 mL to about 4000 mL, about 1500 mL to about 3500 mL, about 1500 mL to about 3000 mL, about 1500 mL to about 2500 mL, about 1500 mL to about 2000 mL, about 2000 mL to about 5000 mL, about 2000 mL to about 4500 mL, about 2000 mL to about 4000 mL, about 2000 mL to about 3500 mL, about 2000 mL to about 3000 mL, about 2000 mL to about 2500 mL, about 2500 mL to about 5000 mL, about 2500 mL to about 4500 mL, about 2500 mL to about 4000 mL, about 2500 mL to about 3500 mL, about 2500 mL to about 3000 mL, about 3000 mL to about 5000 mL, about 3000 mL to about 4500 mL, about 3000 mL to about 4000 mL, about 3000 mL to about 3500 mL, about 3500 mL to about 5000 mL, about 3500 mL to about 4500 mL, about 3500 mL to about 4000 mL, about 4000 mL to about 5000 mL, about 4000 mL to about 4500 mL, or about 4500 mL to about 5000 mL. In certain embodiments, a method described herein results in a total urine output of the human about 8 hours after administration is complete of about 600 mL to about 5300 mL. In certain embodiments, a method described herein results in a total urine output of the human about 8 hours after administration is complete of about 1200 mL to about 5300 mL. In certain embodiments, a method described herein results in a total urine output of the human about 8 hours after administration is complete of about 600 mL to about 2650 mL.

In various embodiments, a method described herein results in a total urine output of the human about 12 hours after administration is complete of about 500 mL to about 5500 mL, about 600 mL to about 5500 mL, about 700 mL to about 5500 mL, about 800 mL to about 5500 mL, about 900 mL to about 5500 mL, about 1000 mL to about 5500 mL, about 1500 mL to about 5500 mL, about 2000 mL to about 5500 mL, about 2500 mL to about 5500 mL, about 3000 mL to about 5500 mL, about 3500 mL to about 5500 mL, about 4000 mL to about 5500 mL, about 4500 mL to about 5500 mL, about 5000 mL to about 5500 mL, about 500 mL to about 5000 mL, about 500 mL to about 4500 mL, about 500 mL to about 4000 mL, about 500 mL to about 3500 mL, about 500 mL to about 3000 mL, about 500 mL to about 2500 mL, about 500 mL to about 2000 mL, about 500 mL to about 1500 mL, about 500 mL to about 1000 mL, about 500 mL to about 900 mL, about 500 mL to about 800 mL, about 500 mL to about 700 mL, about 500 mL to about 600 mL, about 600 mL to about 5000 mL, about 600 mL to about 4500 mL, about 600 mL to about 4000 mL, about 600 mL to about 3500 mL, about 600 mL to about 3000 mL, about 600 mL to about 2500 mL, about 600 mL to about 2000 mL, about 600 mL to about 1500 mL, about 600 mL to about 1000 mL, about 600 mL to about 900 mL, about 600 mL to about 800 mL, about 600 mL to about 700 mL, about 700 mL to about 5000 mL, about 700 mL to about 4500 mL, about 700 mL to about 4000 mL, about 700 mL to about 3500 mL, about 700 mL to about 3000 mL, about 700 mL to about 2500 mL, about 700 mL to about 2000 mL, about 700 mL to about 1500 mL, about 700 mL to about 1000 mL, about 700 mL to about 800 mL, about 800 mL to about 5000 mL, about 800 mL to about 4500 mL, about 800 mL to about 4000 mL, about 800 mL to about 3500 mL, about 800 mL to about 3000 mL, about 800 mL to about 2500 mL, about 800 mL to about 2000 mL, about 800 mL to about 1500 mL, about 800 mL to about 1000 mL, about 800 mL to about 900 mL, about 900 mL to about 5000 mL, about 900 mL to about 4500 mL, about 900 mL to about 4000 mL, about 900 mL to about 3500 mL, about 900 mL to about 3000 mL, about 900 mL to about 2500 mL, about 900 mL to about 2000 mL, about 900 mL to about 1500 mL, about 900 mL to about 1000 mL, about 1000 mL to about 5000 mL, about 1000 mL to about 4500 mL, about 1000 mL to about 4000 mL, about 1000 mL to about 3500 mL, about 1000 mL to about 3000 mL, about 1000 mL to about 2500 mL, about 1000 mL to about 2000 mL, about 1000 mL to about 1500 mL, about 1500 mL to about 5000 mL, about 1500 mL to about 4500 mL, about 1500 mL to about 4000 mL, about 1500 mL to about 3500 mL, about 1500 mL to about 3000 mL, about 1500 mL to about 2500 mL, about 1500 mL to about 2000 mL, about 2000 mL to about 5000 mL, about 2000 mL to about 4500 mL, about 2000 mL to about 4000 mL, about 2000 mL to about 3500 mL, about 2000 mL to about 3000 mL, about 2000 mL to about 2500 mL, about 2500 mL to about 5000 mL, about 2500 mL to about 4500 mL, about 2500 mL to about 4000 mL, about 2500 mL to about 3500 mL, about 2500 mL to about 3000 mL, about 3000 mL to about 5000 mL, about 3000 mL to about 4500 mL, about 3000 mL to about 4000 mL, about 3000 mL to about 3500 mL, about 3500 mL to about 5000 mL, about 3500 mL to about 4500 mL, about 3500 mL to about 4000 mL, about 4000 mL to about 5000 mL, about 4000 mL to about 4500 mL, or about 4500 mL to about 5000 mL. In certain embodiments, a method described herein results in a total urine output of the human about 8 hours after administration is complete of about 600 mL to about 5500 mL. In certain embodiments, a method described herein results in a total urine output of the human about 8 hours after administration is complete of about 1200 mL to about 5500 mL. In certain embodiments, a method described herein results in a total urine output of the human about 8 hours after administration is complete of about 600 mL to about 2750 mL.

### (2) Furosemide Pharmacokinetics

In various embodiments, the methods described herein provide a maximum plasma concentration (Cₘₐₓ) of furosemide in the human of about 1500 ng/ml to about 10000 ng/mL, about 2000 ng/mL to about 10000 ng/mL, about 3000 ng/mL to about 10000 ng/ml, about 4000 ng/ml to about 10000 ng/ml, about 5000 ng/ml to about 10000 ng/mL, about 6000 ng/ml to about 10000 ng/mL, about 7000 ng/ml to about 10000 ng/ml, about 8000 ng/mL to about 10000 ng/mL, about 9000 ng/mL to about 10000 ng/ml, about 1500 ng/ml to about 9000 ng/mL, about 1500 ng/ml to about 8000 ng/mL, about 1500 ng/ml to about 7000 ng/ml, about 1500 ng/ml to about 6000 ng/mL, about 1500 ng/mL to about 5000 ng/ml, about 1500 ng/ml to about 4000 ng/mL, about 1500 ng/ml to about 3000 ng/ml, about 1500 ng/ml to about 2000 ng/mL, about 2000 ng/mL to about 9000 ng/mL, about 2000 ng/ml to about 8000 ng/ml, about 2000 ng/mL to about 7000 ng/mL, about 2000 ng/ml to about 6000 ng/ml, about 2000 ng/ml to about 5000 ng/mL, about 2000 ng/mL to about 4000 ng/ml, about 2000 ng/ml to about 3000 ng/mL, about 3000 ng/mL to about 9000 ng/mL, about 3000 ng/ml to about 8000 ng/mL, about 3000 ng/mL to about 7000 ng/mL, about 3000 ng/ml to about 6000 ng/ml, about 3000 ng/ml to about 5000 ng/mL, about 3000 ng/mL to about 4000 ng/mL, about 4000 ng/ml to about 9000 ng/mL, about 4000 ng/ml to about 8000 ng/mL, about 4000 ng/mL to about 7000 ng/ml, about 4000 ng/mL to about 6000 ng/mL, about 4000 ng/mL to about 5000 ng/mL, about 5000 ng/ml to about 9000 ng/mL, about 5000 ng/mL to about 8000 ng/mL, about 5000 ng/mL to about 7000 ng/mL, about 5000 ng/ml to about 6000 ng/mL, about 6000 ng/mL to about 9000 ng/ml, about 6000 ng/ml to about 8000 ng/mL, about 6000 ng/mL to about 7000 ng/mL, about 7000 ng/ml to about 9000 ng/mL, about 7000 ng/ml to about 8000 ng/mL, or about 8000 ng/ml to about 9000 ng/mL. In certain embodiments, the methods described herein provide a furosemide Cₘₐₓ in plasma of the human of about 1500 ng/mL to about 9800 ng/mL. In certain embodiments, the methods described herein provide a furosemide Cₘₐₓ in plasma of the human of about 3000 ng/ml to about 9800 ng/mL. In certain embodiments, the methods described herein provide a furosemide Cₘₐₓ in plasma of the human of about 1500 ng/ml to about 4900 ng/ml.

In various embodiments, the methods described herein provide a furosemide AUCₗₐₛₜ (e.g., an area under the curve from the time of administration to the last measurable concentration) in plasma of the human of about 4400 h*ng/mL to about 32000 h*ng/ml, about 5000 h*ng/ml to about 32000 h*ng/ml, about 6000 h*ng/mL to about 32000 h*ng/mL, about 7000 h*ng/mL to about 32000 h*ng/mL, about 8000 h*ng/mL to about 32000 h*ng/ml, about 12000 h*ng/mL to about 32000 h*ng/mL, about 16000 h*ng/ml to about 32000 h*ng/ml, about 20000 h*ng/mL to about 32000 h*ng/mL, about 24000 h*ng/mL to about 32000 h*ng/mL, about 28000 h*ng/ml to about 32000 h*ng/mL, about 4400 h*ng/ml to about 28000 h*ng/mL, about 4400 h*ng/mL to about 24000 h*ng/ml, about 4400 h*ng/mL to about 20000 h*ng/ml, about 4400 h*ng/ml to about 16000 h*ng/ml, about 4400 h*ng/ml to about 12000 h*ng/mL, about 4400 h*ng/ml to about 8000 h*ng/ml, about 4400 h*ng/ml to about 7000 h*ng/ml, about 4400 h*ng/mL to about 6000 h*ng/ml, about 4400 h*ng/ml to about 5000 h*ng/ml, about 5000 h*ng/mL to about 28000 h*ng/mL, about 5000 h*ng/ml to about 24000 h*ng/mL, about 5000 h*ng/mL to about 20000 h*ng/ml, about 5000 h*ng/ml to about 16000 h*ng/ml, about 5000 h*ng/mL to about 12000 h*ng/mL, about 5000 h*ng/ml to about 8000 h*ng/mL, about 5000 h*ng/ml to about 7000 h*ng/ml, about 5000 h*ng/ml to about 6000 h*ng/mL, about 6000 h*ng/mL to about 28000 h*ng/ml, about 6000 h*ng/ml to about 24000 h*ng/ml, about 6000 h*ng/ml to about 20000 h*ng/mL, about 6000 h*ng/ml to about 16000 h*ng/ml, about 6000 h*ng/mL to about 12000 h*ng/ml, about 6000 h*ng/ml to about 8000 h*ng/ml, about 6000 h*ng/ml to about 7000 h*ng/ml, about 7000 h*ng/mL to about 28000 h*ng/mL, about 7000 h*ng/ml to about 24000 h*ng/mL, about 7000 h*ng/ml to about 20000 h*ng/ml, about 7000 h*ng/mL to about 16000 h*ng/ml, about 7000 h*ng/mL to about 12000 h*ng/ml, about 7000 h*ng/ml to about 8000 h*ng/mL, about 8000 h*ng/ml to about 28000 h*ng/ml, about 8000 h*ng/mL to about 24000 h*ng/mL, about 8000 h*ng/ml to about 20000 h*ng/mL, about 8000 h*ng/ml to about 16000 h*ng/mL, about 8000 h*ng/ml to about 12000 h*ng/ml, about 12000 h*ng/ml to about 28000 h*ng/mL, about 12000 h*ng/mL to about 24000 h*ng/ml, about 12000 h*ng/ml to about 20000 h*ng/mL, about 12000 h*ng/ml to about 16000 h*ng/ml, about 16000 h*ng/ml to about 28000 h*ng/mL, about 16000 h*ng/ml to about 24000 h*ng/mL, about 16000 h*ng/ml to about 20000 h*ng/mL, about 20000 h*ng/ml to about 28000 h*ng/mL, about 20000 h*ng/mL to about 24000 h*ng/mL, or about 24000 h*ng/mL to about 28000 h*ng/ml. In various embodiments, the methods described herein provide a furosemide AUCₗₐₛₜ in plasma of the human of about 4400 h*ng/ml to about 31700 h*ng/mL. In various embodiments, the methods described herein provide a furosemide AUCₗₐₛₜ in plasma of the human of about 8800 h*ng/ml to about 31700 h*ng/mL. In various embodiments, the methods described herein provide a furosemide AUCₗₐₛₜ in plasma of the human of about 4400 h*ng/ml to about 15850 h*ng/mL.

In various embodiments, the methods described herein provide a furosemide AUC_{inf} (e.g., area under the curve extrapolated to infinity) in plasma of the human of about 4500 h*ng/ml to about 36000 h*ng/mL, about 6000 h*ng/ml to about 36000 h*ng/mL, about 8000 h*ng/ml to about 36000 h*ng/mL, about 12000 h*ng/mL to about 36000 h*ng/mL, about 16000 h*ng/ml to about 36000 h*ng/mL, about 20000 h*ng/ml to about 36000 h*ng/mL, about 24000 h*ng/mL to about 36000 h*ng/mL, about 28000 h*ng/ml to about 36000 h*ng/ml, about 32000 h*ng/ml to about 36000 h*ng/mL, about 4500 h*ng/mL to about 32000 h*ng/ml, about 4500 h*ng/ml to about 28000 h*ng/ml, about 4500 h*ng/ml to about 24000 h*ng/mL, about 4500 h*ng/ml to about 20000 h*ng/mL, about 4500 h*ng/mL to about 16000 h*ng/ml, about 4500 h*ng/ml to about 12000 h*ng/mL, about 4500 h*ng/mL to about 8000 h*ng/ml, about 4500 h*ng/mL to about 6000 h*ng/mL, about 6000 h*ng/ml to about 32000 h*ng/mL, about 6000 h*ng/ml to about 28000 h*ng/mL, about 6000 h*ng/ml to about 24000 h*ng/mL, about 6000 h*ng/mL to about 20000 h*ng/mL, about 6000 h*ng/ml to about 16000 h*ng/ml, about 6000 h*ng/ml to about 12000 h*ng/mL, about 6000 h*ng/ml to about 8000 h*ng/ml, about 8000 h*ng/ml to about 32000 h*ng/mL, about 8000 h*ng/ml to about 28000 h*ng/mL, about 8000 h*ng/ml to about 24000 h*ng/mL, about 8000 h*ng/ml to about 20000 h*ng/ml, about 8000 h*ng/ml to about 16000 h*ng/ml, about 8000 h*ng/ml to about 12000 h*ng/mL, about 12000 h*ng/ml to about 32000 h*ng/mL, about 12000 h*ng/mL to about 28000 h*ng/ml, about 12000 h*ng/mL to about 24000 h*ng/ml, about 12000 h*ng/ml to about 20000 h*ng/ml, about 12000 h*ng/ml to about 16000 h*ng/ml, about 16000 h*ng/ml to about 32000 h*ng/ml, about 16000 h*ng/mL to about 28000 h*ng/mL, about 16000 h*ng/ml to about 24000 h*ng/mL, about 16000 h*ng/ml to about 20000 h*ng/mL, about 20000 h*ng/ml to about 32000 h*ng/mL, about 20000 h*ng/ml to about 28000 h*ng/mL, about 20000 h*ng/mL to about 24000 h*ng/ml, about 24000 h*ng/ml to about 32000 h*ng/mL, about 24000 h*ng/ml to about 28000 h*ng/ml, or about 28000 h*ng/ml to about 32000 h*ng/mL. In certain embodiments, the methods described herein provide a furosemide AUC_{inf} in plasma of the human of about 4500 h*ng/ml to about 33400 h*ng/mL. In certain embodiments, the methods described herein provide a furosemide AUC_{inf} in plasma of the human of about 8900 h*ng/ml to about 33400 h*ng/ml. In certain embodiments, the methods described herein provide a furosemide AUC_{inf} in plasma of the human of about 4500 h*ng/ml to about 16700 h*ng/mL.

### (3) Exemplary Pharmacokinetic/Pharmacodynamic Outcomes

In various embodiments, provided herein is a method of treating a condition selected from the group consisting of congestion due to fluid overload, edema, and hypertension, in a human in need thereof, comprising administering to the human via subcutaneous injection about 80 mg furosemide in a total volume of about 1 mL of a liquid pharmaceutical formulation, wherein one or more of the following:
the method provides a furosemide Cₘₐₓ in plasma of the human of about 3000 ng/mL to about 9800 ng/ml;
the method provides a furosemide AUCₗₐₛₜ in plasma of the human of about 8800 h*ng/ml to about 31700 h*ng/ml;
the method provides a furosemide AUC_{inf} in plasma of the human of about 8900 h*ng/ml to about 33400 h*ng/ml;
the total urine output of the human about 6 hours after administration is complete is about 1200 mL to about 5000 mL;
the total urine output of the human about 8 hours after administration is complete is about 1200 mL to about 5300 mL; and
the total urine output of the human about 12 hours after administration is complete is about 1200 mL to about 5500 mL.

In various embodiments, provided herein is a method of treating a condition selected from the group consisting of congestion due to fluid overload, edema, and hypertension, in a human in need thereof, comprising administering to the human via subcutaneous injection about 80 mg furosemide in a total volume of about 1 mL of a liquid pharmaceutical formulation, wherein:
the method provides a furosemide Cₘₐₓ in plasma of the human of about 3000 ng/ml to about 9800 ng/ml;
the method provides a furosemide AUCₗₐₛₜ in plasma of the human of about 8800 h*ng/ml to about 31700 h*ng/ml;
the method provides a furosemide AUC_{inf} in plasma of the human of about 8900 h*ng/ml to about 33400 h*ng/ml;
the total urine output of the human about 6 hours after administration is complete is about 1200 mL to about 5000 mL;
the total urine output of the human about 8 hours after administration is complete is about 1200 mL to about 5300 mL; and
the total urine output of the human about 12 hours after administration is complete is about 1200 mL to about 5500 mL.

In various embodiments, provided herein is a method of treating a condition selected from the group consisting of congestion due to fluid overload, edema, and hypertension, in a human in need thereof, comprising administering to the human via subcutaneous injection about 40 mg furosemide in a total volume of about 0.5 mL of a liquid pharmaceutical formulation, wherein one or more of the following:
the method provides a furosemide Cₘₐₓ in plasma of the human of about 1500 ng/ml to about 4900 ng/ml;
the method provides a furosemide AUCₗₐₛₜ in plasma of the human of about 4400 h*ng/ml to about 15850 h*ng/ml;
the method provides a furosemide AUC_{inf} in plasma of the human of about 4450 h*ng/mL to about 16700 h*ng/mL;
the total urine output of the human about 6 hours after administration is complete is about 600 mL to about 2500 mL;
the total urine output of the human about 8 hours after administration is complete is about 600 mL to about 2650 mL; and
the total urine output of the human about 12 hours after administration is complete is about 600 mL to about 2750 mL.

In various embodiments, provided herein is a method of treating a condition selected from the group consisting of congestion due to fluid overload, edema, and hypertension, in a human in need thereof, comprising administering to the human via subcutaneous injection about 40 mg furosemide in a total volume of about 0.5 mL of a liquid pharmaceutical formulation, wherein:
the method provides a furosemide Cₘₐₓ in plasma of the human of about 1500 ng/ml to about 4900 ng/ml;
the method provides a furosemide AUCₗₐₛₜ in plasma of the human of about 4400 h*ng/ml to about 15850 h*ng/ml;
the method provides a furosemide AUC_{inf} in plasma of the human of about 4450 h*ng/ml to about 16700 h*ng/ml;
the total urine output of the human about 6 hours after administration is complete is about 600 mL to about 2500 mL;
the total urine output of the human about 8 hours after administration is complete is about 600 mL to about 2650 mL; and
the total urine output of the human about 12 hours after administration is complete is about 600 mL to about 2750 mL.

In various embodiments, provided herein is a method of treating a condition selected from the group consisting of congestion due to fluid overload, edema, and hypertension, in a human in need thereof, comprising administering to the human via subcutaneous injection about 2 mg bumetanide in a total volume of about 1 mL of a liquid pharmaceutical formulation, wherein one or more of the following:
the total urine output of the human about 6 hours after administration is complete is about 1200 mL to about 5000 mL;
the total urine output of the human about 8 hours after administration is complete is about 1200 mL to about 5300 mL; and
the total urine output of the human about 12 hours after administration is complete is about 1200 mL to about 5500 mL.

In various embodiments, provided herein is a method of treating a condition selected from the group consisting of congestion due to fluid overload, edema, and hypertension, in a human in need thereof, comprising administering to the human via subcutaneous injection about 2 mg bumetanide in a total volume of about 1 mL of a liquid pharmaceutical formulation, wherein:
the total urine output of the human about 6 hours after administration is complete is about 1200 mL to about 5000 mL;
the total urine output of the human about 8 hours after administration is complete is about 1200 mL to about 5300 mL; and
the total urine output of the human about 12 hours after administration is complete is about 1200 mL to about 5500 mL.

In various embodiments, provided herein is a method of treating a condition selected from the group consisting of congestion due to fluid overload, edema, and hypertension, in a human in need thereof, comprising administering to the human via subcutaneous injection about 1 mg bumetanide in a total volume of about 0.5 mL of a liquid pharmaceutical formulation, wherein one or more of the following:
the total urine output of the human about 6 hours after administration is complete is about 600 mL to about 2500 mL;
the total urine output of the human about 8 hours after administration is complete is about 600 mL to about 2650 mL; and
the total urine output of the human about 12 hours after administration is complete is about 600 mL to about 2750 mL.

In various embodiments, provided herein is a method of treating a condition selected from the group consisting of congestion due to fluid overload, edema, and hypertension, in a human in need thereof, comprising administering to the human via subcutaneous injection about 1 mg bumetanide in a total volume of about 0.5 mL of a liquid pharmaceutical formulation, wherein:
the total urine output of the human about 6 hours after administration is complete is about 600 mL to about 2500 mL;
the total urine output of the human about 8 hours after administration is complete is about 600 mL to about 2650 mL; and
the total urine output of the human about 12 hours after administration is complete is about 600 mL to about 2750 mL.

In various embodiments, provided herein is a method of treating a condition selected from the group consisting of congestion due to fluid overload, edema, and hypertension, in a human in need thereof, comprising administering to the human via subcutaneous injection about 40 mg torsemide in a total volume of about 1 mL of a liquid pharmaceutical formulation, wherein one or more of the following:
the total urine output of the human about 6 hours after administration is complete is about 1200 mL to about 5000 mL;
the total urine output of the human about 8 hours after administration is complete is about 1200 mL to about 5300 mL; and
the total urine output of the human about 12 hours after administration is complete is about 1200 mL to about 5500 mL.

In various embodiments, provided herein is a method of treating a condition selected from the group consisting of congestion due to fluid overload, edema, and hypertension, in a human in need thereof, comprising administering to the human via subcutaneous injection about 40 mg torsemide in a total volume of about 1 mL of a liquid pharmaceutical formulation, wherein:
the total urine output of the human about 6 hours after administration is complete is about 1200 mL to about 5000 mL;
the total urine output of the human about 8 hours after administration is complete is about 1200 mL to about 5300 mL; and
the total urine output of the human about 12 hours after administration is complete is about 1200 mL to about 5500 mL.

In various embodiments, provided herein is a method of treating a condition selected from the group consisting of congestion due to fluid overload, edema, and hypertension, in a human in need thereof, comprising administering to the human via subcutaneous injection about 20 mg torsemide in a total volume of about 0.5 mL of a liquid pharmaceutical formulation, wherein one or more of the following:
the total urine output of the human about 6 hours after administration is complete is about 600 mL to about 2500 mL;
the total urine output of the human about 8 hours after administration is complete is about 600 mL to about 2650 mL; and
the total urine output of the human about 12 hours after administration is complete is about 600 mL to about 2750 mL.

In various embodiments, provided herein is a method of treating a condition selected from the group consisting of congestion due to fluid overload, edema, and hypertension, in a human in need thereof, comprising administering to the human via subcutaneous injection about 20 mg torsemide in a total volume of about 0.5 mL of a liquid pharmaceutical formulation, wherein:
the total urine output of the human about 6 hours after administration is complete is about 600 mL to about 2500 mL;
the total urine output of the human about 8 hours after administration is complete is about 600 mL to about 2650 mL; and
the total urine output of the human about 12 hours after administration is complete is about 600 mL to about 2750 mL.

### (C) Delivery Devices

In certain embodiments, the liquid pharmaceutical formulation is administered from a needle and syringe set, an autoinjector, a single use fixed dose injection pen, a multiuse fixed dose injection pen, a single use variable dose injection pen, or a multiuse variable dose injection pen. In certain embodiments, the liquid pharmaceutical formulation is administered from a needle and syringe set. In certain embodiments, the liquid pharmaceutical formulation is administered from an autoinjector. In some embodiments, the autoinjector is a disposable autoinjector. In certain embodiments, the liquid pharmaceutical formulation is administered from a single use fixed dose injection pen. In certain embodiments, the liquid pharmaceutical formulation is administered from a multiuse fixed dose injection pen. In certain embodiments, the liquid pharmaceutical formulation is administered from a single use variable dose injection pen. In certain embodiments, the liquid pharmaceutical formulation is administered from a multiuse variable dose injection pen.

### (D) Time of Administration

In certain embodiments, the liquid pharmaceutical formulations described herein are administered to the patient by subcutaneous injection over a period of equal to or less than 30 secs (e.g., 1 sec, 2 secs, 3 secs, 4 secs, 5 secs, 6 secs, 7 secs, 8 secs, 9 secs, 10 secs, 11 secs, 12 secs, 13 secs, 14 secs, 15 secs, 16 secs, 17 secs, 18 secs, 19 secs, 20 secs, 21 secs, 22 secs, 23 secs, 24 secs, 25 secs, 26 secs, 27 secs, 28 secs, 29 secs, or 30 secs).

### (E) Patients

In certain embodiments, the human is an adult human. In certain embodiments, the adult human has New York Heart Association (NYHA) Class II, Class III or Class IV chronic heart failure, liver disease or impairment, or renal disease or impairment. In certain embodiments, the adult human displays reduced responsiveness to oral diuretics before beginning subcutaneous administration of a loop diuretic according to a method described herein.

In certain embodiments, the adult human is 30 to 80 years of age, 35 to 80 years of age, 40 to 80 years of age, 45 to 80 years of age, 50 to 80 years of age, 55 to 80 years of age, 60 to 80 years of age, 65 to 80 years of age, 70 to 80 years of age, 75 to 80 years of age, 30 to 75 years of age, 30 to 70 years of age, 30 to 65 years of age, 30 to 60 years of age, 30 to 55 years of age, 30 to 50 years of age, 30 to 45 years of age, 30 to 40 years of age, 30 to 35 years of age, 35 to 75 years of age, 35 to 70 years of age, 35 to 65 years of age, 35 to 60 years of age, 35 to 55 years of age, 35 to 50 years of age, 35 to 45 years of age, 35 to 40 years of age, 40 to 75 years of age, 40 to 70 years of age, 40 to 65 years of age, 40 to 60 years of age, 40 to 55 years of age, 40 to 50 years of age, 40 to 45 years of age, 45 to 75 years of age, 45 to 70 years of age, 45 to 65 years of age, 45 to 60 years of age, 45 to 55 years of age, 45 to 50 years of age, 50 to 75 years of age, 50 to 70 years of age, 50 to 65 years of age, 50 to 60 years of age, 50 to 55 years of age, 55 to 75 years of age, 55 to 70 years of age, 55 to 65 years of age, 55 to 60 years of age, 60 to 75 years of age, 60 to 70 years of age, 60 to 65 years of age, 65 to 75 years of age, 65 to 70 years of age, or 70 to 75 years of age. In certain embodiments, the adult human is 30 to 80 years of age. In certain embodiments, the adult human is 45 to 80 years of age.

In certain embodiments, the adult human is 30, 31, 32, 33, 34, 35, 36, 37, 38, 39, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 years of age.

When administered for the treatment or prevention of a disease or disorder disclosed herein, it may be understood that an effective dosage can vary depending upon many factors such as the particular compound or therapeutic combination utilized, the mode of administration, and severity of the condition being treated, as well as the various physical factors related to the individual being treated. In therapeutic applications, a liquid pharmaceutical formulation described herein may be provided to a patient already suffering from said disease or disorder in an amount sufficient to cure or at least partially ameliorate the symptoms of the disease or disorder and its complications. The dosage to be used in the treatment of a specific individual typically must be subjectively determined by an attending physician. The variables involved include the specific condition and state as well as the size, age and response pattern of the patient. In some embodiments, the amount sufficient to cure or at least partially ameliorate the symptoms of the disease or disorder and its complications is an effective amount. In some embodiments, the amount sufficient to cure or at least partially ameliorate the symptoms of the disease or disorder and its complications is an effective amount.

### Kits

In one aspect, the invention provides kits for the treatment or prevention of a variety of conditions such as, but not limited to, congestion due to fluid overload, edema, and hypertension in a patient in need thereof. In certain embodiments, the condition, disease, or disorder is selected from the group consisting of congestion due to fluid overload, edema, and hypertension. In certain embodiments, the condition is congestion due to fluid overload. In certain embodiments, the condition is edema. In certain embodiments, the condition is hypertension. In certain embodiments, the edema is associated with congestive heart failure, cirrhosis of the liver or renal disease. In certain embodiments, the renal disease is nephrotic syndrome.

In various embodiments, a kit comprises a liquid pharmaceutical formulation described herein. In certain embodiments, the kit comprises one or more unit doses of a liquid pharmaceutical formulation described herein. In certain embodiments, the kit further comprises a medical device. In certain embodiments, the kit further comprises instructions for treating a disease or disorder of the present invention.

A number of medical devices have been proposed to facilitate self-administration of a liquid pharmaceutical formulation. The device may include a reservoir containing, for example, pre-loaded with, a liquid pharmaceutical formulation described herein to be administered. A type of device useful for the intramuscular or subcutaneous delivery of pharmaceutical formulations is an autoinjector. In various embodiments, the kit comprises an autoinjector.

Accordingly, in various embodiments, a medical device such as a micropump or patch device can include a reservoir containing a pharmaceutical formulation, a subcutaneous injection needle configured for removable insertion into skin of a patient, a micropump having an inlet in fluid communication with the reservoir and an outlet in fluid communication with the subcutaneous injection needle, a control system configured for controlling the micropump to deliver the pharmaceutical formulation from the reservoir to the subcutaneous injection needle, whereby the pharmaceutical formulation is administered subcutaneously to a patient, and a housing for supporting the reservoir, subcutaneous injection needle, micropump and control system, the housing being portable and adapted for contact with the skin of the patient. The liquid pharmaceutical formulation contained within the reservoir can be any of the liquid pharmaceutical formulations or the unit liquid pharmaceutical formulations described herein.

In certain embodiments, the medical device can be of a unitary construction. Such medical devices can be for a single or one-time use. In particular embodiments, the medical device can be of a multi-piece construction. In such medical devices, a disposable or a reusable portion or component can be present. For example, a housing defining or including the reservoir can be a disposable or a reusable component of the medical device. In some embodiments, the disposable or reusable housing defining or including the reservoir can contain a pharmaceutical formulation of the present teachings. In various embodiments, the subcutaneous injection needle can be a disposable component of the medical device.

In certain embodiments, the medical device is selected from the group comprising a needle and syringe set, an autoinjector, a single use fixed dose injection pen, a multiuse fixed dose injection pen, a single use variable dose injection pen, or a multiuse variable dose injection pen.

### EXAMPLES

The invention now being generally described, will be more readily understood by reference to the following examples, which are merely for the purposes of illustration of certain aspects and embodiments of the present invention, and are not intended to limit the invention.

### Example 1. Manufacturing Process for a Furosemide Liquid Pharmaceutical Formulation

In the following example, the manufacturing process for preparing all exemplified furosemide liquid pharmaceutical formulations.

The corresponding amounts of the ingredients are provided in the formulas disclosed in Table 1.

Tromethamine was added to an appropriate amount of water for injection (WFI) and mixed until dissolved. Sodium hydroxide (NaOH) was added. Approximately ⅓ of total amount of drug substance, furosemide, was added and mixed until dissolved. Benzyl alcohol was added and mixed until homogenous. NaOH was added. Approximately ⅓ of total amount of drug substance, furosemide, was added and mixed until dissolved. NaOH was added. The remainder of drug substance, furosemide, was added and mixed until dissolved. A pH sample was taken and the solution adjusted with additional NaOH or HCl to a pH of 7.20 to 7.60. After pH adjustment, WFI was added to the resulting solution to achieve the final weight and mixed. The solution was filtered through a 0.22 μm PVDF filter and filled into glass syringes.

TABLE 1
Formula for Furosemide Liquid Pharmaceutical Formulations

| | Formulation |
|---|---|
| Component | (pH = 6.5-8.5) |
| Furosemide | 60-120 mg/mL |
| Tris Buffer | 5-15 mg/mL |
| Benzyl Alcohol | 25-45 mg/mL |
| Hydrochloric Acid | qs |
| Sodium Hydroxide | qs |
| Water | qs |

### Example 2. Study to Compare the Pharmacokinetics and Pharmacodynamics of a High Concentration/Low Volume Furosemide Liquid Pharmaceutical Formulation Administered as a Subcutaneous Injection Vs Furosemide Administered as an Intravenous Injection in Healthy Volunteers

### (1) Study Objectives

Primary Objective: estimate the bioavailability of a high concentration/low volume furosemide liquid pharmaceutical formulation (80 mg furosemide in 1 mL), referred to herein in this Example as SCP-111, administered as a subcutaneous injection via an autoinjector compared with an equivalent dose of furosemide administered as two 40 mg intravenous injections over two minutes, two hours apart.

Secondary Objectives: (1) describe the pharmacokinetics and pharmacodynamics of SCP-111 administered as a subcutaneous injection via an autoinjector; and (2) describe the safety and tolerability of SCP-111 administered as a subcutaneous injection via an autoinjector.

### (2) End Points

Primary Endpoints: logarithmic transformed geometric mean ratio between subcutaneous SCP-111 and 80 mg intravenous furosemide of the: (1) AUC from time 0 (pre-dose) to the last quantifiable time point (AUCₗₐₛₜ); and (2) AUC from time 0 (pre-dose) extrapolated to infinity (AUC_{inf}).

Secondary Endpoints: the following pharmacokinetics (PK) parameters will be evaluated for subcutaneous and intravenous furosemide administration: (1) maximum observed plasma concentration (Cₘₐₓ); (2) time to Cₘₐₓ (Tₘₐₓ); (3) terminal phase elimination rate constant (λz), estimated by linear regression of logarithmically transformed concentration versus time data; (4) terminal elimination half-life (t_{1/2}) estimated using the equation [ln(2)/λz]; (5) apparent systemic clearance (CL/F); (6) systemic clearance (CL); (7) apparent systemic volume of distribution (Vz/F); and (8) systemic volume of distribution (V).

The following pharmacodynamics (PD) parameters are evaluated for subcutaneous and intravenous furosemide administration: (1) urine output (0-6, 0-8 hours and 0-12); (2) urinary sodium excretion (0-6, 0-8 hours and 0-12); and (3) urinary potassium excretion (0-6, 0-8 hours and 0-12).

Adverse events (AEs) and serious adverse events (SAEs) are grouped by body system and summarized. The incidence (number and percentage of subjects) of adverse events and serious adverse events are presented overall and by MedDRA System Organ Class and Preferred Term.

### (3) Study Duration

3 months.

### (4) Study Design and Methodology

This is an open-label, single-center, single-dose, randomized, two-way (two-period) crossover study in healthy volunteers. Each subject completes the screening, baseline, treatment, and follow-up phases.

The screening phase is conducted on an outpatient basis between 14 and 3 days prior to the baseline visit. Subjects are instructed to maintain a<2-gram sodium diet within 2 days prior to admission to the clinical research unit (CRU). On arrival to the CRU (Day 0), baseline and final qualification assessments are performed.

The treatment phase comprises two crossover periods separated by a 2-4 day washout period. Following each CRU admission, subjects continue on a<2-gram sodium diet through discharge. Subjects are randomly assigned in a 1:1 ratio to 1 of 2 treatment sequences to receive intravenous (IV) furosemide or subcutaneous (SC) SCP-111 in crossover periods (i.e., IV followed by SC or vice versa). Plasma is collected to measure furosemide concentrations at pre-dose and for 12-hours post-dose. Prior to dosing in each treatment phase, subjects void their bladder completely and urine is collected starting after the injection through 12-hours post injection. Subjects remain domiciled in the CRU for each treatment phase through 12 hours after administration of study drug. After final assessments are performed, subjects are discharged from the CRU if safety parameters are acceptable to the Investigator.

The follow-up phase occurs 24-48 hours (Day 5±1) after discharge from the CRU following crossover period 2, completing subjects' study participation.

### (5) Study Treatments

Intravenous furosemide (IV furosemide): Hospira, Furosemide Injection, Solution 10 mg/mL (NDA 018667) (total dose=80 mg) administered intravenously. 40 mg over 2 minutes by IV injection followed by a second dose of 40 mg over 2 minutes, 2 hours later (reference treatment).

SCP-111 (furosemide injection): total dose=80 mg; administered as a subcutaneous injection (test treatment) via an autoinjector.

Study Drug: SCP-111, (Furosemide Injection), 80 mg/1 mL is a furosemide liquid pharmaceutical formulation that is buffered to a neutral pH for subcutaneous administration.

Study Device: BD Intevia™ delivery system is a disposable autoinjector composed of two sub-assemblies combined with BD Neopak™ 1 mL long prefilled syringe ½ inch staked-in needle covered by a rigid needle shield (RNS) closure and BD FluroTec® 1 mL stopper.

SCP-111 Autoinjector is an investigational drug-device combination product consisting of SCP-111 and a commercially available 2-step disposable autoinjector (BD Intevia™ 1.0 mL Disposable Autoinjector).

### (6) Subject Population

Number of subjects in the study: 18.

Subjects are enrolled in the study only if all the inclusion criteria and none of the exclusion criteria are met.

### Inclusion Criteria:

Female and male subjects are eligible for inclusion only if all the following criteria are met: (1) an Institutional Review Board (IRB) approved informed consent is signed and dated prior to any study-related activities; (2) male and female subjects are 18 years of age or older; (3) the subjects have the ability to understand the requirements of the study and is willing to comply with all study procedures; and (4) in the opinion of the Investigator, a subject is able to participate in the study.

### Exclusion Criteria:

A subject is not eligible for inclusion if any of the following criteria apply: (1) pregnant or lactating women or women of childbearing age who are not willing to use an adequate form of contraception; (2) systolic BP (SBP)<90 mmHg at screening or baseline; (3) heart rate>110 beats per minute (BPM) at screening or baseline; (4) temperature>38° C. (oral or equivalent); (5) serum potassium<3.0 or >5.5 mEq/L at screening; (6) other significant cardiac abnormalities which may interfere with study participation or study assessments; (7) current or planned treatment during the study with any IV therapies, including inotropic agents, vasopressors, levosimendan, nesiritide or analogues; (8) presence of implanted ventricular assist device, cardiac defibrillator or pacemaker; (9) severely impaired renal function, defined as an estimated glomerular filtration rate (eGFR) at screening admission<30 mL/min/1.73 m², calculated using the simplified Modification of Diet in Renal Disease (sMDRD) equation; (10) urinary retention due to bladder emptying disorders and/or urethral narrowing; (11) presence or need for urinary catheterization; (12) history of liver disease, cirrhosis, or ascites; (13) moderate-to-severe hepatic dysfunction as determined by the investigator; (14) administration of intravenous radiographic contrast agent within 72 hours prior to screening; (15) concomitant use of drugs known to interact with furosemide (aminoglycoside antibiotics, ethacrynic acid, high doses of salicylates, cisplatin, tubocurarine, succinylcholine, chloral hydrate, phenytoin, methotrexate, indomethacin, or lithium); (16) administration of an investigational drug or implantation of investigational device, or participation in another interventional clinical trial, within 30 days prior to screening; (17) any surgical or medical condition which in the opinion of the investigator may interfere with participation in the study or which may affect the outcome of the study; (18) positive urine drug screen at screening or baseline; (19) blood alcohol concentration>2 mg/dL (0.02%) at screening; (20) alcohol breath test>2 mg/dL (0.02%) on admission to the CRU; and (21) history of severe allergic or hypersensitivity reactions to furosemide.

### (7) Pharmacokinetic and Statistical Analysis

Pharmacokinetic parameters are estimated using noncompartmental methods employing Phoenix® WinNonlin® V8.1 or higher. Individual pharmacokinetic parameters for furosemide are calculated using noncompartmental analysis and are summarized using descriptive statistics. Bioavailability of SCP-111 is determined by the logarithmic transformed geometric mean ratios of the SCP-111 to IV furosemide AUCₗₐₛₜ and AUC_{inf} and 90% confidence intervals for the geometric mean ratios are determined and should be within 80% and 125% to establish bioequivalence.

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

What is claimed:

1. A method of treating a condition selected from the group consisting of congestion due to fluid overload, edema, and hypertension, in a human in need thereof, comprising administering to the human via subcutaneous injection an effective amount of a loop diuretic in a total volume of about 1 mL or less of a liquid pharmaceutical formulation.

2. The method of claim 1, wherein the effective amount of the loop diuretic in the total volume of the liquid pharmaceutical formulation is about 1 mg to about 100 mg.

3. The method of claim 1 or 2, wherein the concentration of the loop diuretic in the total volume of the liquid pharmaceutical formulation is about 2 mg/mL to about 100 mg/mL.

4. The method of any one of claims 1-3, wherein the total urine output of the human about 6 hours after administration is complete is about 600 mL to about 5000 mL.

5. The method of any one of claims 1-4, wherein the total urine output of the human about 8 hours after administration is complete is about 600 mL to about 5300 mL.

6. The method of any one of claims 1-5, wherein the total urine output of the human about 12 hours after administration is complete is about 600 mL to about 5500 mL.

7. The method of any one of claims 1-6, wherein the loop diuretic is bumetanide, furosemide, or torsemide.

8. A method of treating a condition selected from the group consisting of congestion due to fluid overload, edema, and hypertension, in a human in need thereof, comprising administering to the human via subcutaneous injection an effective amount of furosemide in a total volume of about 1 mL or less of a liquid pharmaceutical formulation.

9. The method of claim 8, wherein the effective amount of furosemide in the total volume of the liquid pharmaceutical formulation is about 40 mg to about 80 mg.

10. The method of claim 8 or 9, wherein the concentration of furosemide in the total volume of the liquid pharmaceutical formulation is about 80 mg/mL.

11. The method of any one of claims 8-10, wherein the total volume of the liquid pharmaceutical formulation is about 0.5 mL or about 1 mL.

12. A method of treating a condition selected from the group consisting of congestion due to fluid overload, edema, and hypertension, in a human in need thereof, comprising administering to the human via subcutaneous injection about 80 mg of furosemide in a total volume of about 1 mL of a liquid pharmaceutical formulation.

13. A method of treating a condition selected from the group consisting of congestion due to fluid overload, edema, and hypertension, in a human in need thereof, comprising administering to the human via subcutaneous injection about 40 mg of furosemide in a total volume of about 0.5 mL of a liquid pharmaceutical formulation.

14. The method of any one of claims 8-13, wherein the method provides a furosemide Cₘₐₓ in plasma of the human of about 1500 ng/ml to about 9800 ng/mL.

15. The method of any one of claims 8-14, wherein the method provides a furosemide AUCₗₐₛₜ in plasma of the human of about 4400 h*ng/ml to about 31700 h*ng/mL.

16. The method of any one of claims 8-15, wherein the method provides a furosemide AUC_{inf} in plasma of the human of about 4500 h*ng/ml to about 33400 h*ng/mL.

17. The method of any one of claims 8-16, wherein the total urine output of the human about 6 hours after administration is complete is about 600 mL to about 5000 mL.

18. The method of any one of claims 8-17, wherein the total urine output of the human about 8 hours after administration is complete is about 600 mL to about 5300 mL.

19. The method of any one of claims 8-18, wherein the total urine output of the human about 12 hours after administration is complete is about 600 mL to about 5500 mL.

20. A method of treating a condition selected from the group consisting of congestion due to fluid overload, edema, and hypertension, in a human in need thereof, comprising administering to the human via subcutaneous injection about 80 mg furosemide in a total volume of about 1 mL of a liquid pharmaceutical formulation, wherein one or more of the following:
the method provides a furosemide Cₘₐₓ in plasma of the human of about 3000 ng/ml to about 9800 ng/ml;
the method provides a furosemide AUCₗₐₛₜ in plasma of the human of about 8800 h*ng/ml to about 31700 h*ng/ml;
the method provides a furosemide AUC_{inf} in plasma of the human of about 8900 h*ng/ml to about 33400 h*ng/ml;
the total urine output of the human about 6 hours after administration is complete is about 1200 mL to about 5000 mL;
the total urine output of the human about 8 hours after administration is complete is about 1200 mL to about 5300 mL; and
the total urine output of the human about 12 hours after administration is complete is about 1200 mL to about 5500 mL.

21. A method of treating a condition selected from the group consisting of congestion due to fluid overload, edema, and hypertension, in a human in need thereof, comprising administering to the human via subcutaneous injection about 80 mg furosemide in a total volume of about 1 mL of a liquid pharmaceutical formulation, wherein:
the method provides a furosemide Cₘₐₓ in plasma of the human of about 3000 ng/ml to about 9800 ng/ml;
the method provides a furosemide AUCₗₐₛₜ in plasma of the human of about 8800 h*ng/ml to about 31700 h*ng/ml;
the method provides a furosemide AUC_{inf} in plasma of the human of about 8900 h*ng/ml to about 33400 h*ng/ml;
the total urine output of the human about 6 hours after administration is complete is about 1200 mL to about 5000 mL;
the total urine output of the human about 8 hours after administration is complete is about 1200 mL to about 5300 mL; and
the total urine output of the human about 12 hours after administration is complete is about 1200 mL to about 5500 mL.

22. A method of treating a condition selected from the group consisting of congestion due to fluid overload, edema, and hypertension, in a human in need thereof, comprising administering to the human via subcutaneous injection about 40 mg furosemide in a total volume of about 0.5 mL of a liquid pharmaceutical formulation, wherein one or more of the following:
the method provides a furosemide Cₘₐₓ in plasma of the human of about 1500 ng/ml to about 4900 ng/mL;
the method provides a furosemide AUCₗₐₛₜ in plasma of the human of about 4400 h*ng/ml to about 15850 h*ng/ml;
the method provides a furosemide AUC_{inf} in plasma of the human of about 4500 h*ng/ml to about 16700 h*ng/mL;
the total urine output of the human about 6 hours after administration is complete is about 600 mL to about 2500 mL;
the total urine output of the human about 8 hours after administration is complete is about 600 mL to about 2650 mL; and
the total urine output of the human about 12 hours after administration is complete is about 600 mL to about 2750 mL.

23. A method of treating a condition selected from the group consisting of congestion due to fluid overload, edema, and hypertension, in a human in need thereof, comprising administering to the human via subcutaneous injection about 40 mg furosemide in a total volume of about 0.5 mL of a liquid pharmaceutical formulation, wherein:
the method provides a furosemide Cₘₐₓ in plasma of the human of about 1500 ng/ml to about 4900 ng/ml;
the method provides a furosemide AUCₗₐₛₜ in plasma of the human of about 4400 h*ng/ml to about 15850 h*ng/ml;
the method provides a furosemide AUC_{inf} in plasma of the human of about 4450 h*ng/ml to about 16700 h*ng/ml;
the total urine output of the human about 6 hours after administration is complete is about 600 mL to about 2500 mL;
the total urine output of the human about 8 hours after administration is complete is about 600 mL to about 2650 mL; and
the total urine output of the human about 12 hours after administration is complete is about 600 mL to about 2750 mL.

24. The method of any one of claims 1-23, wherein administering comprises administering the total volume over a period of equal to or less than 30 secs.

25. The method of any one of claims 1-24, wherein administering comprises administering the total volume from an autoinjector.

26. The method of any one of claims 1-25, wherein the liquid pharmaceutical formulation further comprises a pharmaceutically acceptable buffer, optionally wherein the pharmaceutically acceptable buffer comprises tromethamine, or a pharmaceutically acceptable salt thereof.

27. The method of any one of claims 1-26, wherein the liquid pharmaceutical formulation has a pH of about 7.2 to about 8.

28. The method of any one of claims 1-27, wherein the liquid pharmaceutical formulation further comprises one or more pharmaceutically acceptable excipients, optionally wherein the one or more pharmaceutically acceptable excipients is selected from benzyl alcohol, sodium hydroxide, sodium chloride, hydrochloric acid, and combinations thereof.

29. The method of any one of claims 1-28, wherein the human is an adult human, optionally wherein the adult human is 45 to 80 years of age.

30. The method of claim 29, wherein the adult human has New York Heart Association (NYHA) Class II, Class III, or Class IV chronic heart failure, liver disease or impairment, or renal disease or impairment.

31. The method of claim 29 or 30, wherein the adult human displays reduced responsiveness to oral diuretics before beginning subcutaneous administration according to the method.

32. The method of any one of claims 1-31, wherein the edema is associated with congestive heart failure, cirrhosis of the liver, or renal disease, optionally wherein the renal disease is nephrotic syndrome.
